SEQUENCE LISTING (16 sequences)

SEQ ID NO: 1 - DNA, 37 bp, Artificial Sequence
  Description of Artificial Sequence Synthetic primer
ggggacaagtttgtacaaaaaagcaggctacaaaatg

SEQ ID NO: 2 - DNA, 33 bp, Artificial Sequence
  Description of Artificial Sequence Synthetic primer
ggggaccactttgtacaagaaagctgggtttta

SEQ ID NO: 3 - DNA, 432 bp, Unknown
  Description of Unknown Rhinovirus 2A protease sequence
atgggcccctctgacatgtatgtgcatgtgggaaaccttatatacagaaacctgcacttgttcaatagtgagatgcacgagagtatcctcgtttcctacagcagtgacttgattatctacaggactaataccgtgggcgatgactacataccgtcatgtgactgcacacaggccacctactattgtaagcacaaaaaccgctattttcccatcactgtaacgtcccatgattggtacgagattcaggaaagcgagtattatcccaaacacatccaatacaacttgttgattggagaaggcccttgtgagcctggggactgtggtggcaagctcttgtgcaaacacggagtcatcggtatagtcactgcgggaggcgataaccatgtcgcgttcattgacctgcgacatttccactgcgccgaggaacagtaa

SEQ ID NO: 4 - DNA, 453 bp, Unknown
  Description of Unknown Poliovirus 2A protease sequence
atggggttcgggcaccagaacaaggcagtttatactgccggctacaagatctgtaactatcaccttgcaacgcaggatgacttgcaaaatgctgtcaatgtcatgtggagccgcgaccttttggtaactgagtcacgcgcccaggggacagatagtatagctaggtgtaattgcaacgctggagtttattattgcgagtcacgccggaaatactatccagtgagtttcgtggggcctacttttcagtatatggaggctaacaactactacccagcgcggtatcaaagccacatgcttatcggccatgggtttgccagccctggtgattgtggaggcatactccgctgccatcacggggtaatcggcattattacggctggtggtgaaggtctggtcgcgttctctgacattagagatctttacgcatatgaggaagaagcgatggaacagtaa

SEQ ID NO: 5 - DNA, 303 bp, Human immunodeficiency virus 1
atgccccaaataacactctggcaaagaccccttgtcactatcaagattggaggacagctcaaggaggcgttgctcgacacaggggcggacgacactgttttggaggagatgtctttgccgggacggtggaagccgaagatgattggagggatcggtggctttattaaggtgagacaatacgatcaaatcctgatcgagatatgcggtcacaaggcgattgggacggtacttgtagggcctactcccgtgaacataatcgggcgcaaccttcttacacagattggttgtacattgaatttttaa

SEQ ID NO: 6 - DNA, 303 bp, Human immunodeficiency virus 2
atgccgcaattctccctgtggaaaaggcctgttgtgaccgcgtatatcgaggggcaaccagttgaagttctcctggacactggggctgatgactccatcgtcgcaggtattgaattgggaaataactactcccctaagattgtggggggcataggagggttcatcaacacaaaggagtacaagaatgttgaaatcgaggtattgaacaaaaaagtcagagcaaccattatgacaggcgatacgccaatcaacatatttggtcgaaacatcttgacggctttgggtatgtcactcaatctctaa

SEQ ID NO: 7 - DNA, 2355 bp, Escherichia coli
atgaatcctgagcgttctgaacgcattgaaatccccgtattgccgctgcgcgatgtggtggtttatccgcacatggtcatccccttatttgtcgggcgggaaaaatctatccgttgtctggaagcggcgatggaccatgataaaaaaattatgctggtcgcgcagaaagaagcttcaacggatgagccgggtgtaaacgatcttttcaccgtcgggaccgtggcctctatattgcagatgctgaaactgcctgacggcaccgtcaaagtgctggtcgaggggttacagcgcgcgcgtatttctgcgctctctgacaatggcgaacacttttctgcgaaggcggagtatctggagtcgccgaccattgatgagcgggaacaggaagtgctggtgcgtactgcaatcagccagttcgaaggctacatcaagctgaacaaaaaaatcccaccagaagtgctgacgtcgctgaatagcatcgacgatccggcgcgtctggcggataccattgctgcacatatgccgctgaaactggctgacaaacagtctgttctggagatgtccgacgttaacgaacgtctggaatatctgatggcaatgatggaatcggaaatcgatctgctgcaggttgagaaacgcattcgcaaccgcgttaaaaagcagatggagaaatcccagcgtgagtactatctgaacgagcaaatgaaagctattcagaaagaactcggtgaaatggacgacgcgccggacgaaaacgaagccctgaagcgcaaaatcgacgcggcgaagatgccgaaagaggcaaaagagaaagcggaagcagagttgcagaagctgaaaatgatgtctccgatgtcggcagaagcgaccgtagtgcgtggttatatcgactggatggtacaggtgccgtggaatgcgcgtagcaaggtcaaaaaagacctgcgtcaggcgcaggaaatccttgataccgaccattatggtctggagcgcgtgaaagatcgaatccttgagtatcttgcggttcaaagccgtgtcaacaaaatcaagggaccgatcctctgcctggtagggccgccgggggtaggtaaaacctctcttggtcagtccattgccaaagccaccgggcgtaaatatgtccgtatggcgctgggcggcgtgcgtgatgaagcggaaatccgtggtcaccgccgtacttacatcggttctatgccgggtaaactgatccagaaaatggcgaaagtgggcgtgaaaaacccgctgttcctgctcgatgagatcgacaaaatgtcttctgacatgcgtggcgatccggcctctgcactgcttgaagtgctggatccagagcagaacgtagcgttcagcgaccactacctggaagtggattacgatctcagcgacgtgatgtttgtcgcgacgtcgaactccatgaacattccggcaccgctgctggatcgtatggaagtgattcgcctctccggttataccgaagatgaaaaactgaacatcgccaaacgtcacctgctgccgaagcagattgaacgtaatgcactgaaaaaaggtgagctgaccgtcgacgatagcgccattatcggcattattcgttactacacccgtgaggcgggcgtgcgtggtctggagcgtgaaatctccaaactgtgtcgcaaagcggttaagcagttactgctcgataagtcattaaaacatatcgaaattaacggcgataacctgcatgactatctcggtgttcagcgtttcgactatggtcgcgcggataacgaaaaccgtgtcggtcaggtaaccggtctggcgtggacggaagtgggcggtgacttgctgaccattgaaaccgcatgtgttccgggtaaaggcaaactgacctataccggttcgctcggcgaagtgatgcaggagtccattcaggcggcgttaacggtggttcgtgcgcgtgcggaaaaactggggatcaaccctgatttttacgaaaaacgtgacatccacgtccacgtaccggaaggtgcgacgccgaaagatggtccgagtgccggtattgctatgtgcaccgcgctggtttcttgcctgaccggtaacccggttcgtgccgatgtggcaatgaccggtgagatcactctgcgtggtcaggtactgccgatcggtggtttgaaagaaaaactcctggcagcgcatcgcggcgggattaaaacagtgctaattccgttcgaaaataaacgcgatctggaagagattcctgacaacgtaattgccgatctggacattcatcctgtgaagcgcattgaggaagttctgactctggcgctgcaaaatgaaccgtctggtatgcaggttgtgactgcaaaatag

SEQ ID NO: 8 - DNA, 1011 bp, Staphylococcus aureus
atgaaaggtaaatttttaaaagttagttctttattcgttgcaactttgacaacagcgacacttgtgagttctccagcagcaaacgcgttatcttcaaaggctatggacaatcatccacaacaaacgcagtcaagcaaacagcaaacacctaagattcaaaaaggcggtaaccttaaaccattagaacaacgtgaacacgcaaatgttatattaccaaataacgatcgtcaccaaatcacagatacaacgaatggtcattatgcacccgtaacttatattcaagttgaagcacctactggtacatttattgcttccggtgtagttgtaggtaaagatactcttttaacaaataaacacgtcgtagatgctacgcacggtgatcctcatgctttaaaagcattcccttctgcaattaaccaagacaattatccaaatggtggtttcactgctgaacaaatcactaaatattcaggcgaaggtgatttagcaatagttaaattctcccctaatgagcaaaacaaacatattggtgaagtagttaaaccagcaacaatgagtaataatgctgaaacacaagttaaccaaaatattactgtaacaggatatcctggtgataaacctgtagcaacaatgtgggaaagtaaaggaaaaatcacttacctcaaaggcgaagctatgcaatatgatttaagtacaactggtggtaattcaggttcacctgtatttaatgaaaaaaatgaagtgatcggaattcattggggcggtgtaccaaatgaatttaatggtgcggtatttattaatgaaaatgtacgcaacttcttaaaacaaaatattgaagatatccattttgccaacgatgaccaacctaataacccagataatcctgataaccctaacaatcctgataaccctaacaacccagatgaaccaaataaccctgacaaccctaacaaccctgataatccagacaatggcgataacaataattcagacaatccagatgcagcttaa

SEQ ID NO: 9 - DNA, 906 bp, Staphylococcus aureus
atggcagcaactggcacaggtaaaggtgtgcttggagatacaaaagatatcaatatcaatagtattgatggtggatttagtttagaggatttgacgcatcaaggtaaattatcagcatacaattttaacgatcaaacaggtcaagcgacattaattactaatgaagatgaaaacttcgtcaaagatgatcaacgtgctggtgtagatgcgaattattatgctaaacaaacatatgattactacaaaaatacatttggtcgtgagtcttacgataaccatggtagtccaatagtctcattaacacatgtaaatcattatggtggacaagataacagaaataacgctgcatggattggagacaaaatgatttatggtgatggcgatggccgcacgtttacaaatttatcaggtgcaaatgacgtagtagcacatgagttaacacatggcgtgacacaagaaacggcgaatttagagtataaagatcaatctggtgcgttaaatgaaagcttttcagatgtttttggatactttgtagatgatgaggatttcttgatgggtgaagatgtttacacaccaggaaaagagggagatgctttacgaagcatgtcaaacccagaacaatttggtcaaccatctcatatgaaagactatgtatacactgaaaaagataacggtggtgtgcatacgaattctggcattccaaataaagcagcttataacgtaattcaagcaatagggaaatctaaatcagaacaaatttactaccgagcattaacggaatacttaacaagtaattcaaacttcaaagattgtaaagatgcattataccaagcggctaaagatttatatgacgagcaaacagctgaacaagtatatgaagcatggaacgaagttggcgtcgagtaa

SEQ ID NO: 10 - DNA, 1883 bp, Vibrio cholerae
atgagcaatgcttttttataatgccaactttgtacaaaaaagcaggctacaaaatgaaaatgatacaacgtcctctgaattggttagttctggccggagcggcaactggcttccctctctatgcggcacaaatggtcacgattgatgatgcatcaatggttgaacaagcgttggcgcagcaacagtacagtatgatgcctgccgccagcggttttaaagccgtcaatacggtacagttgccgaatggtaaggtgaaagtgcgttaccagcagatgtacaacggggttcctgtctatggcaccgttgtggtggcaaccgagtccagtaaagggatttcgcaagtgtatggtcaaatggctcagcagttggaagccgatctcccaaccgtgacccctgacattgaaagccagcaggccatcgctttagcggttagccattttggtgaacaacacgctggagaatcgctcccggtggaaaacgaaagtgtgcaactgatggtacgtttggatgataaccaacaggctcagttagtgtacttggtcgacttttttgtcgcttcagaaacaccttcgcgtccgttctactttatcagtgcggaaacgggagaagtgctagaccaatgggatggcattaaccacgcacaggcaacaggaaccggccccggcggtaaccaaaaaacgggacgttatgaatacggcagtaacggtttacccggtttcacgattgataagaccggaaccacctgtacgatgaataacagtgcggtaaaaaccgttaacctcaatggcggcacctcgggtagcacggcgttcagttatgcttgtaacaacagcactaactacaacagcgtgaaaacagtgaatggtgcttactcaccgcttaacgacgcgcacttctttggaaaagtggtgtttgatatgtatcagcagtggttgaatacttcgccgctgactttccaattaaccatgcgtgtgcactacggcaataactatgaaaatgccttctgggatggacgcgccatgacttttggtgatggctatacccgtttctatcctttggtggatatcaacgttagtgcccatgaggtcagccacggttttactgagcagaattcaggcctcgtttaccgagatatgtccggtggtattaacgaagcattctcggatatcgcaggggaagcggcagagtactttatgcgtggcaatgtcgactggattgtcggcgcggatatttttaaatcctccggtggtctacgttatttcgatcagccgtcacgtgatggccgctcgatagatcatgcttcacagtattacagcggtattgatgttcaccattcgagtggcgtgtttaaccgcgcgttttacctactcgccaataaatcgggttggaacgtacgtaaaggttttgaagtgtttgccgtggctaaccagttgtactggacaccgaacagcacgtttgatcaaggtggctgtggggtagtgaaagcggcgcaggatctcaactacaacaccgcagacgttgtggcagcctttaataccgtgggtgtcaatgcttcttgtggcaccacgccaccacctgtcggcaaagtgcttgagaaaggtaaaccgatcacaggactgagcggctcacgtggaggagaagatttctatacctttacggtgaccaattcaggcagtgttgttgtgtccatcagtggtggaacgggcgatgcggatctgtatgtcaaagcgggcagcaaacccaccacctcttcttgggattgtcgtccataccgttcaggcaatgccgagcagtgttccatctctgcggtcgtgggtacgacataccatgtcatgttacgcggttacagtaactattctggtgtgacgttacgcttggactaa

SEQ ID NO: 11 - DNA, 1536 bp, Human immunodeficiency virus
atgaagtgccttttgtacttagcctttttattcattggggtgaattgcaagttcaccatagtttttccacacaaccaaaaaggaaactggaaaaatgttccttctaattaccattattgcccgtcaagctcagatttaaattggcataatgacttaataggcacagccttacaagtcaaaatgcccaagagtcacaaggctattcaagcagacggttggatgtgtcatgcttccaaatgggtcactacttgtgatttccgctggtatggaccgaagtatataacacattccatccgatccttcactccatctgtagaacaatgcaaggaaagcattgaacaaacgaaacaaggaacttggctgaatccaggcttccctcctcaaagttgtggatatgcaactgtgacggatgccgaagcagtgattgtccaggtgactcctcaccatgtgctggttgatgaatacacaggagaatgggttgattcacagttcatcaacggaaaatgcagcaattacatatgccccactgtccataactctacaacctggcattctgactataaggtcaaagggctatgtgattctaacctcatttccatggacatcaccttcttctcagaggacggagagctatcatccctgggaaaggagggcacagggttcagaagtaactactttgcttatgaaactggaggcaaggcctgcaaaatgcaatactgcaagcattggggagtcagactcccatcaggtgtctggttcgagatggctgataaggatctctttgctgcagccagattccctgaatgcccagaagggtcaagtatctctgctccatctcagacctcagtggatgtaagtctaattcaggacgttgagaggatcttggattattccctctgccaagaaacctggagcaaaatcagagcgggtcttccaatctctccagtggatctcagctatcttgctcctaaaaacccaggaaccggtcctgctttcaccataatcaatggtaccctaaaatactttgagaccagatacatcagagtcgatattgctgctccaatcctctcaagaatggtcggaatgatcagtggaactaccacagaaagggaactgtgggatgactgggcaccatatgaagacgtggaaattggacccaatggagttctgaggaccagttcaggatataagtttcctttatacatgattggacatggtatgttggactccgatcttcatcttagctcaaaggctcaggtgttcgaacatcctcacattcaagacgctgcttcgcaacttcctgatgatgagagtttattttttggtgatactgggctatccaaaaatccaatcgagcttgtagaaggttggttcagtagttggaaaagctctattgcctcttttttctttatcatagggttaatcattggactattcttggttctccgagttggtatccatctttgcattaaattaaagcacaccaagaaaagacagatttatacagacatagagatgaaccgacttggaaagtaa

SEQ ID NO: 12 - DNA, 1149 bp, Human immunodeficiency virus
atgagggacaattggagaagtgaattatataaatataaagtagtaaaaattgaaccattaggagtagcacccaccaaggcaaagagaagagtggtgcagagagaaaaaagagcagtgggaataggagctttgttccttgggttcttgggagcagcaggaagcactatgggcgcagcgtcaatgacgctgacggtacaggccagacaattattgtctggtatagtgcagcagcagaacaatttgctgagggctattgaggcgcaacagcatctgttgcaactcacagtctggggcatcaagcagctccaggcaagaatcctggctgtggaaagatacctaaaggatcaacagctcctggggatttggggttgctctggaaaactcatttgcaccactgctgtgccttggaatgctagttggagtaataaatctctggaacagatttggaatcacacgacctggatggagtgggacagagaaattaacaattacacaagcttaatacactccttaattgaagaatcgcaaaaccagcaagaaaagaatgaacaagaattattggaattagataaatgggcaagtttgtggaattggtttaacataacaaattggctgtggtatataaaattattcataatgatagtaggaggcttggtaggtttaagaatagtttttgctgtactttctatagtgaatagagttaggcagggatattcaccattatcgtttcagacccacctcccaaccccgaggggacccgacaggcccgaaggaatagaagaagaaggtggagagagagacagagacagatccattcgattagtgaacggatccttggcacttatctgggacgatctgcggagcctgtgcctcttcagctaccaccgcttgagagacttactcttgattgtaacgaggattgtggaacttctgggacgcagggggtgggaagccctcaaatattggtggaatctcctacaatattggagtcaggagctaaagaatagtgctgttagcttgctcaatgccacagccatagcagtagctgaggggacagatagggttatagaagtagtacaaggagcttgtagagctattcgccacatacctagaagaataagacagggcttggaaaggattttgctataa

SEQ ID NO: 13 - DNA, 1503 bp, Human immunodeficiency virus
atgggtgcgagagcgtcagtattaagcgggggagaattagatcgatgggaaaaaattcggttaaggccagggggaaagaaaaaatataaattaaaacatatagtatgggcaagcagggagctagaacgattcgcagttaatcctggcctgttagaaacatcagaaggctgtagacaaatactgggacagctacaaccatcccttcagacaggatcagaagaacttagatcattatataatacagtagcaaccctctattgtgtgcatcaaaggatagagataaaagacaccaaggaagctttagacaagatagaggaagagcaaaacaaaagtaagaaaaaagcacagcaagcagcagctgacacaggacacagcaatcaggtcagccaaaattaccctatagtgcagaacatccaggggcaaatggtacatcaggccatatcacctagaactttaaatgcatgggtaaaagtagtagaagagaaggctttcagcccagaagtgatacccatgttttcagcattatcagaaggagccaccccacaagatttaaacaccatgctaaacacagtggggggacatcaagcagccatgcaaatgttaaaagagaccatcaatgaggaagctgcagaatgggatagagtgcatccagtgcatgcagggcctattgcaccaggccagatgagagaaccaaggggaagtgacatagcaggaactactagtacccttcaggaacaaataggatggatgacacataatccacctatcccagtaggagaaatctataaaagatggataatcctgggattaaataaaatagtaagaatgtatagccctaccagcattctggacataagacaaggaccaaaggaaccctttagagactatgtagaccgattctataaaactctaagagccgagcaagcttcacaagaggtaaaaaattggatgacagaaaccttgttggtccaaaatgcgaacccagattgtaagactattttaaaagcattgggaccaggagcgacactagaagaaatgatgacagcatgtcagggagtggggggacccggccataaagcaagagttttggctgaagcaatgagccaagtaacaaatccagctaccataatgatacagaaaggcaattttaggaaccaaagaaagactgttaagtgtttcaattgtggcaaagaagggcacatagccaaaaattgcagggcccctaggaaaaagggctgttggaaatgtggaaaggaaggacaccaaatgaaagattgtactgagagacaggctaattttttagggaagatctggccttcccacaagggaaggccagggaattttcttcagagcagaccagagccaacagccccaccagaagagagcttcaggtttggggaagagacaacaactccctctcagaagcaggagccgatagacaaggaactgtatcctttagcttccctcagatcactctttggcagcgacccctcgtcacaataa

SEQ ID NO: 14 - DNA, 873 bp, Human immunodeficiency virus
atgtttttagatggaatagataaggcccaagaagaacatgagaaatatcacagtaattggagagcaatggctagtgattttaacctaccacctgtagtagcaaaagaaatagtagccagctgtgataaatgtcagctaaaaggggaagccatgcatggacaagtagactgtagcccaggaatatggcagctagattgtacacatttagaaggaaaagttatcttggtagcagttcatgtagccagtggatatatagaagcagaagtaattccagcagagacagggcaagaaacagcatacttcctcttaaaattagcaggaagatggccagtaaaaacagtacatacagacaatggcagcaatttcaccagtactacagttaaggccgcctgttggtgggcggggatcaagcaggaatttggcattccctacaatccccaaagtcaaggagtaatagaatctatgaataaagaattaaagaaaattataggacaggtaagagatcaggctgaacatcttaagacagcagtacaaatggcagtattcatccacaattttaaaagaaaaggggggattggggggtacagtgcaggggaaagaatagtagacataatagcaacagacatacaaactaaagaattacaaaaacaaattacaaaaattcaaaattttcgggtttattacagggacagcagagatccagtttggaaaggaccagcaaagctcctctggaaaggtgaaggggcagtagtaatacaagataatagtgacataaaagtagtgccaagaagaaaagcaaagatcatcagggattatggaaaacagatggcaggtgatgattgtgtggcaagtagacaggatgaggattaataa

SEQ ID NO: 15 - DNA, 1328 bp, Escherichia coli
atgagcaatgcttttttataatgccaactttgtacaaaaaagcaggctacaaaatgacagataaacgcaaagatggctcaggcaaattgctgtattgctctttttgcggcaaaagccagcatgaagtgcgcaagctgattgccggtccatccgtgtatatctgcgacgaatgtgttgatttatgtaacgacatcattcgcgaagagattaaagaagttgcaccgcatcgtgaacgcagtgcgctaccgacgccgcatgaaattcgcaaccacctggacgattacgttatcggccaggaacaggcgaaaaaagtgctggcggtcgcggtatacaaccattacaaacgtctgcgcaacggcgataccagcaatggcgtcgagttgggcaaaagtaacattctgctgatcggtccgaccggttccggtaaaacgctgctggctgaaacgctggcgcgcctgctggatgttccgttcaccatggccgacgcgactacactgaccgaagccggttatgtgggtgaagacgttgaaaacatcattcagaagctgttgcagaaatgcgactacgatgtccagaaagcacagcgtggtattgtctacatcgatgaaatcgacaagatttctcgtaagtcagacaacccgtccattacccgagacgtttccggtgaaggcgtacagcaggcactgttgaaactgatcgaaggtacggtagctgctgttccaccgcaaggtgggcgtaaacatccgcagcaggaattcttgcaggttgatacctctaagatcctgtttatttgtggcggtgcgtttgccggtctggataaagtgatttcccaccgtgtagaaaccggctccggcattggttttggcgcgacggtaaaagcgaagtccgacaaagcaagcgaaggcgagctgctggcgcaggttgaaccggaagatctgatcaagtttggtcttatccctgagtttattggtcgtctgccggttgtcgcaacgttgaatgaactgagcgaagaagctctgattcagatcctcaaagagccgaaaaacgccctgaccaagcagtatcaggcgctgtttaatctggaaggcgtggatctggaattccgtgacgaggcgctggatgctatcgctaagaaagcgatggcgcgtaaaaccggtgcccgtggcctgcgttccatcgtagaagccgcactgctcgataccatgtacgatctgccgtccatggaagacgtcgaaaaagtggttatcgacgagtcggtaattgatggtcaaagcaaaccgttgctgatttatggcaagccggaagcgcaacaggcatctggtgaataa

SEQ ID NO: 16 - DNA, 3135 bp, Homo sapiens
atgggaatgctggagctgcggatcaagaacctgacccagagccggagcaccacctggaagctgaacaacctgctgctgaacgactactgggttcacaacgagatgaaggccgagatcaagatgttcttcgagaccaacgagaacaaggacaccacctaccagaacctgtgggacgccttcaaggccgtgtgccggggcaagttcatcgccctgaacgcctacaagcggaagcaggagcggagcaagatcgacaccctgaccagccagctgaaggagctggagaagcaggagcagacccacagcaaggccagtcggcggcaggagatcaccaagatccgggccgagctgaaggagatcgagacccagaagacccttcagaagatcaacgagagccggagctggttcttcgagcggatcaacaagatcgaccggcccctggcccggctgatcaagaagaagcgggagaagaaccagatcgacaccatcaagaacgacaagggcgacatcaccaccgaccccaccgagatccagaccaccatccgggagtactacaagcacctgtacgccaacaagctggagaatctagaggagatggacaccttcctggacacctacaccctgccccggctgaaccaggaggaggtggagagcctgaaccggcccatcaccggcagcgagatcgtggccatcatcaacagcctgcccaccaagaagagccccggccccgacggcttcaccgccgagttctaccagcggtacaaggaggagctggtgcccttcctgctgaagctgttccagagcatcgagaaggagggcatcctgcccaacagcttctacgaggccagcatcatcctgatccccaagcccggccgggacaccaccaagaaggagaacttccggcccatcagcctgatgaacatcgacgccaagatcctgaacaagatcctggccaaccggattcagcagcacatcaagaagctgatccaccacgaccaagttggcttcatccccggcatgcaaggatggttcaacatccggaagagcatcaacgtgatccagcacatcaaccgggccaaggacaagaaccacatgatcatcagcatcgacgccgagaaggctttcgacaagatccagcagcccttcatgctgaagaccctgaacaagctgggcatcgacggcacctacttcaagatcatccgggccatctacgacaagcccaccgccaacatcatcctgaacggccagaagctggaggctttccccctgaagaccggcacccggcagggctgccccctgagccccctgctgttcaacatcgtgctggaggtgctggcccgggccatccggcaggagaaggagatcaagggcatccagctgggcaaggaggaggtgaagctgagcctgttcgccgacgacatgatcgtgtacctggagaaccctattgttagcgcccagaacctgctgaagctgatcagcaacttcagtaaagttagcggctacaagatcaacgtgcagaagagccaggctttcctgtacaccaataaccggcagaccgagagccagatcatgggcgagctgcccttcaccatcgccagcaagcggatcaagtacctgggcatccagctgactcgggacgtcaaggacctgttcaaggagaactacaagcccctgctgaaggagatcaaggaggagaccaacaagtggaagaacatcccctgtagctgggtgggccggatcaacatcgtgaagatggccatcctgccgaaagttatctatcggttcaacgccatccccatcaagctgcccatgaccttcttcaccgagctggagaagaccaccctgaagttcatctggaaccagaagcgggcccggatcgccaagagcatcctgagccagaagaacaaggccggcggcatcaccctgcccgacttcaagctgtactacaaggccaccgtgaccaagaccgcctggtactggtaccagaaccgggacatcgaccagtggaaccggaccgagcccagcgagatcatgccccacatctacaactacctgatcttcgacaagcccgagaagaacaagcagtggggcaaggacagcctgttcaacaagtggtgctgggagaactggctggccatctgccggaagctgaagctggaccccttcctgaccccctacaccaagatcaacagccggtggatcaaggacctgaacgtgaagcccaagaccatcaagaccctggaggagaacctgggcatcaccatccaggacatcggcgtgggcaaggacttcatgagcaagacccccaaggccatggccaccaaggacaagatcgacaagtgggacctgatcaagctgaagagcttctgcaccgccaaggagaccaccatccgggtgaaccggcagcccaccacctgggagaagattttcgccacctacagcagcgacaagggcctgatcagccggatctacaacgagctgaagcagatttacaagaagaagaccaacaaccccatcaagaagtgggccaaggacatgaaccggcacttcagcaaggaggacatctacgccgccaagaagcacatgaagaagtgcagcagcagcctggccatccgggagatgcagatcaagaccaccatgcggtatcacctgacccccgtgcggatggccatcatcaagaagagcggcaacaaccgatgctggcggggctgcggcgagatcggcaccctgctgcactgctggtgggactgcaagctggtgcagcccctgtggaagagcgtgtggcggttcctgcgggacctggagctggagatccccttcgaccccgccatccccctgctgggcatctaccccaacgagtacaagagctgctgctacaaggacacctgcacccggatgttcatcgccgccctgttcaccatcgccaagacctggaaccagcccaagtgccccaccatgatcgactggatcaagaagatgtggcacatctacactatggagtactacgccgccatcaagaacgacgagttcatcagcttcgtgggcacctggatgaagctggagaccatcatcctgagcaagctgagccaggagcagaagaccaagcaccggatcttcagcctgatcggcggcaactaa

(19) United States
(12) Patent Application Publication
(10) Pub. No.: US 2021/0324379 A1
(43) Pub. Date: Oct. 21, 2021

(54) METHODS OF DRUG SCREENING USING DNA BARCODING

(71) Applicant: The Trustees of Columbia University in the City of New York, New York, NY (US)

(72) Inventors: Alejandro Chavez, New York, NY (US); Seo Jung Hong, New York, NY (US); Samuel Resnick, New York, NY (US)

(73) Assignee: The Trustees of Columbia University in the City of New York, New York, NY (US)

(21) Appl. No.: 17/243,931

(22) Filed: Apr. 29, 2021

Related U.S. Application Data
(63) Continuation of application No. PCT/US2019/059906, filed on Nov. 5, 2019.
(60) Provisional application No. 62/755,822, filed on Nov. 5, 2018.
(60) Provisional application No. 62/886,576, filed on Aug. 14, 2019.

(51) Int. Cl. C12N 15/10

(52) U.S. Cl. CPC C12N 15/1075; C12N 15/1082

(57) ABSTRACT

The present disclosure provides methods for high-throughput, multiplexed drug screening to identify effective and specific drugs, as well as effective drug concentrations.

### RELATED APPLICATIONS

This application is a continuation application which claims the benefit of priority to PCT/US2019/059906, filed on Nov. 5, 2019, which in turn claims the benefit of priority to U.S. Provisional Patent Application No. 62/755,822, filed Nov. 5, 2018, and U.S. Provisional Patent Application No. 62/886,576, filed Aug. 14, 2019. The content of each of these applications is incorporated herein by reference in their entirety.

### SEQUENCE LISTING

The instant application contains a Sequence Listing which has been submitted electronically in ASCII format and is hereby incorporated by reference in its entirety. Said ASCII copy, created on Apr. 26, 2021, is named 123756_01103_SL.txt and is 25,114 bytes in size.

### BACKGROUND

Current methods of performing drug screens, including small molecule screens, are based on taking a single target protein of interest and exposing it to a library of agents, e.g., small molecules. This approach, while effective, is costly, labor intensive, and has low information content as upwards of a million dollars and months of labor are spent to identify drugs against a single target. In addition, a common reason for failure in small molecule development is a lack of target specificity, which is unaccounted for by an approach where only one target protein is screened. Finally, due to issues of cost and labor, most screens are performed at a single drug concentration. This limits users from identifying compounds that work at concentrations outside those tested, or recognizing those that are likely to be very safe as they remain innocuous to the cell even at high doses. Thus, there is a need for a multiplexed, high-throughput method to screen drugs and to identify effective and specific drugs, as well as effective drug concentrations.

### SUMMARY

The present disclosure relates, at least in part, to methods for high-throughput, multiplexed drug screening.

In some aspects, the present disclosure provides methods of screening for a perturbation, e.g., an agent, capable of specifically inhibiting a toxicity protein in a cell comprising (a) providing a library comprising a plurality of proliferating cell types, wherein each cell type comprises one or more inducible toxicity protein, wherein each proliferating cell type comprises a unique associated barcode, and wherein the library further comprises one or more control cell types selected from: (1) a proliferating cell type comprising a negative control non-toxic inducible protein, and (2) a proliferating cell type comprising a positive control inducible toxicity protein, wherein each control cell type comprises a unique associated control barcode; (b) inducing the expression of each toxicity protein and the one or more control proteins in the presence or absence of at least one perturbation; (c) determining the relative number of unique associated barcodes and the unique associated control barcodes after a period of cell proliferation; and (d) comparing the relative number of unique associated barcodes to one or more of the unique barcodes associated control barcodes, to thereby determine the effectiveness and/or specificity of the perturbation on the one or more toxicity proteins.

In some aspects, the present disclosure provides methods of identifying an effective dosage of a drug having the ability to inhibit cellular toxicity in a cell, comprising (a) providing a library comprising a plurality of proliferating cell types, wherein each cell type comprises one or more inducible toxicity protein and one or more cell surface drug transporter, and wherein each proliferating cell type comprises a unique associated barcode, and wherein the library further comprises one or more control cell types selected from: (1) a proliferating cell type comprising a negative control non-toxic inducible protein, and (2) a proliferating cell type comprising a positive control inducible toxicity protein, wherein each control cell type comprises a unique associated control barcode; (b) inducing the expression of each toxicity protein and inducing varying levels of expression of the one or more cell surface drug transporters, in the presence or absence of a single concentration of at least one drug, wherein the proliferating cell types contain different intracellular concentrations of the at least one drug; (c) determining the relative number of unique associated barcodes after a period of cell proliferation; and (d) comparing the relative number of unique associated barcodes to one or more controls, to thereby determine the effective intracellular concentration of the at least one drug on the one or more proliferating cell types.

In some embodiments, the one or more cell surface drug transporters are drug exporters. In some embodiments, the one or more cell surface drug transporters are drug importers. In some embodiments, the one or more cell surface drug transporters are endogenous to the cell. In other embodiments, the one or more cell surface drug transporters are exogenous to the cell.

In some embodiments, the foregoing methods further comprise monitoring for off-target effects of the perturbation, comprising (a) providing one or more additional proliferating cell types, wherein each additional proliferating cell type comprising a unique associated barcode within its genome, and wherein the one or more additional proliferating cell types are highly sensitive to cellular insults, (b) exposing the one or more additional proliferating cell types to the perturbation, (c) determining the relative number of unique barcodes associated with the one or more additional proliferating cell types, and (d) identifying the perturbation as causing one or more off-target effects when the number of barcodes associated with the one or more additional proliferating cell types are depleted as compared to the number of barcodes associated with one or more control cell types that are not highly sensitive to cellular insults.

In some embodiments, each of the steps of the methods described herein are performed concurrently.

In some embodiments, one or more additional proliferating cell types comprise cells that have been modified to be highly sensitive to one or more cellular insults. In some embodiments, the one or more proliferating cell types highly sensitive to cellular insults are cells that carry mutations or deletions in one or more genes selected from the group consisting of RAD, LEA1, CHO2, RFM1, LSM1, HOC1, ROM2, HAC1, SMY1, ABP1, ERV14, SNT1, PFA4, SSD1, GSF2, and CLB2.

In some embodiments, each proliferating cell type further comprises one or more cell surface drug transporter, and the method further comprises inducing varying levels of expression of the one or more cell surface drug transporters.

In some embodiments, the perturbation is a drug perturbation, an environmental perturbation, or a genetic perturbation. In some embodiments, the drug perturbation is a small molecule or a biologic, e.g., a protein or a peptide, an antibody, or a nucleic acid.

In some embodiments, the toxicity protein is any protein which induces toxicity in the cell. In some embodiments, the toxicity protein is a kinase, protease, aggregation-prone protein, viral integrase, nucleic acid binding protein, structural protein, protein chaperone, phosphatase, small GTPase, ubiquitin ligase, DNA or RNA polymerase, caspase, hydrolase, ligase, oxidoreductase, transcription factor, cell adhesion molecule, cell junction molecule, isomerase, transferase, adapter protein, or reverse transcriptase.

In some embodiments, the cell types are any proliferating cell types capable of expressing a phenotype associated with a toxicity protein expressed in the cell. In some embodiments, the cell types are eukaryotic or prokaryotic cell types. In some embodiments, the cell types are yeast cells, fungal cells, insect cells, bacterial cells or mammalian cells. In some embodiments, the yeast cells are Saccharomyces cerevisiae.

In some aspects, the present disclosure provides a method of screening for an agent capable of specifically inhibiting a toxicity protein in a cell, comprising (a) providing a library comprising a plurality of proliferating cell types in the presence or absence of the agent, wherein each cell type comprises one or more inducible toxicity protein, wherein each proliferating cell type comprises a unique associated barcode, and wherein the library further comprises one or more control cell types selected from: (1) a proliferating cell type comprising a negative control inducible non-toxic protein, (2) a proliferating cell type comprising a positive control inducible toxicity protein, and (3) one or more proliferating cell types highly sensitive to cellular insults, wherein each control cell type comprises a unique associated control barcode; (b) inducing the expression of each toxicity protein and the one or more control proteins; (c) determining the relative number of unique associated barcodes and the unique associated control barcodes after a period of cell proliferation; and (d) comparing the relative number of unique associated barcodes to one or more of the unique associated control barcodes, to thereby determine the effectiveness and specificity of the agent on the one or more toxicity proteins.

In some embodiments, the method further comprises step (e) pooling results of step (d) from the cell types comprising the same combination of the inducible toxicity protein and the level of expression of the cell surface drug transporter. In some embodiments, each of the foregoing method steps are performed concurrently.

In some embodiments, each proliferating cell type further comprises one or more cell surface drug transporter having varying levels of expression for identifying an effective dosage of the agent having the ability to inhibit cellular toxicity. In some embodiments, the one or more cell surface drug transporters are drug exporters. In some embodiments, the one or more cell surface drug transporters are drug importers. In some embodiments, the one or more cell surface drug transporters are endogenous to the cell. In other embodiments, the one or more cell surface drug transporters are exogenous to the cell.

In some embodiments, each combination of an inducible toxicity protein, one or more controls, and a level of expression of a cell surface drug transporter is associated with a unique barcode within the cell type comprising said combination.

In some embodiments, the agent is a small molecule.

In some embodiments, the toxicity protein is a kinase, a protease, an aggregation-prone protein, a viral integrase, a nucleic acid binding protein, a structural protein, a protein chaperone, phosphatase, small GTPase, ubiquitin ligase, DNA or RNA polymerase, caspase, hydrolase, ligase, oxidoreductase, transcription factor, cell adhesion molecule, cell junction molecule, isomerase, transferase, adapter protein, or a reverse transcriptase.

In some embodiments, the cell types are eukaryotic or prokaryotic cell types. In some embodiments, the cell types are yeast cells.

In other aspects, the present disclosure provides methods of identifying an effective dosage of a drug having the ability to inhibit cellular toxicity in a cell, comprising (a) providing a library comprising a plurality of proliferating cell types, wherein each cell type comprises one or more inducible toxicity protein and having varying levels of expression of one or more cell surface drug transporter, and wherein each proliferating cell type comprises a unique associated barcode, and wherein the library further comprises one or more control cell types selected from: (1) a proliferating cell type comprising a negative control inducible non-toxic protein, and (2) a proliferating cell type comprising a positive control inducible toxicity protein, wherein each control cell type comprises a unique associated control barcode; (b) inducing the expression of each toxicity protein in the presence or absence of a single concentration of the drug, wherein the proliferating cell types contain different intracellular concentrations of the drug as a result of the varying levels of expression of one or more cell surface drug transporter; (c) determining the relative number of unique associated barcodes after a period of cell proliferation; and (d) comparing the relative number of unique associated barcodes to one or more controls, to thereby determine the effective intracellular concentration of the drug on the one or more proliferating cell types.

In some embodiments, the method further comprises step (e) pooling results of step (d) from the cell types comprising the same combination of the inducible toxicity protein and the level of expression of the cell surface drug transporter.

In some embodiments, the one or more cell surface drug transporters are drug exporters. In some embodiments, the one or more cell surface drug transporters are drug importers. In some embodiments, the one or more cell surface drug transporters are endogenous to the cell. In other embodiments, the one or more cell surface drug transporters are exogenous to the cell.

In some embodiments, each combination of an inducible toxicity protein, one or more controls, and a level of expression of a cell surface drug transporter is associated with a unique barcode within the cell type comprising said combination. In some embodiments, each of the foregoing steps are performed concurrently.

In some embodiments, the one or more additional proliferating cell types are cells that have been modified to be highly sensitive to one or more cellular insults. In some embodiments, the one or more proliferating cell types highly sensitive to cellular insults are cells that carry mutations or deletions in one or more genes selected from the group consisting of RAD, LEA1, CHO2, RFM1, LSM1, HOC1, ROM2, HAC1, SMY1, ABP1, ERV14, SNT1, PFA4, SSD1, GSF2, and CLB2.

In some embodiments, the agent is a small molecule.

In some embodiments, the toxicity protein is a kinase, a protease, an aggregation-prone protein, a viral integrase, a nucleic acid binding protein, a structural protein, a protein chaperone, phosphatase, small GTPase, ubiquitin ligase, DNA or RNA polymerase, caspase, hydrolase, ligase, oxidoreductase, transcription factor, cell adhesion molecule, cell junction molecule, isomerase, transferase, adapter protein, or a reverse transcriptase.

In some embodiments, the cell types are eukaryotic or prokaryotic cell types. In some embodiments, the cell types are yeast cells.

### BRIEF DESCRIPTION OF DRAWINGS

FIG. 1: Spot assays of yeast expressing pAG426-GAL empty vector or pAG426-GAL plasmids encoding various viral and bacterial proteases. Strains were grown to saturation in SC-ura glucose liquid suspension and serial dilutions (10-fold) of yeast were plated on SC-ura glucose or SC-ura galactose plates.

FIG. 2: Spot assays of yeast expressing pAG426-GAL empty vector or pAG426-GAL plasmids encoding various proteases and their respective catalytically dead mutants. Strains were grown to saturation in SC-ura glucose liquid suspension and serial dilutions (10-fold) of yeast were plated on SC-ura galactose plates.

FIG. 3: Spot assays of yeast expressing pAG426-GAL empty vector or pAG426-GAL plasmids encoding various viral, bacterial, and human proteins. Strains were grown to saturation in SC-ura glucose media and serial dilutions (10-fold) of yeast were plated on SC-ura glucose or SC-ura galactose plates.

FIG. 4: Spot assays of yeast expressing pAG426-GAL empty vector or pAG426-GAL plasmids encoding HIV integrase or the protein chaperone ClpX. Point mutations are designed to disrupt the natural catalytic function of these proteins. Strains were grown to saturation in SC-ura glucose media and serial dilutions (10-fold) of yeast were plated on SC-ura galactose plates.

FIG. 5: Spot assays of Δpdr1 Δpdr3 Δsnq2 yeast expressing pAG426-GAL empty vector or pAG426-GAL-HIV protease. Strains were grown to saturation in SC-ura glucose media and serial dilutions (10-fold) of yeast were plated on SC-ura glucose or SC-ura galactose plates with or without 5 μM lopinavir added.

FIG. 6: DNA barcoding strategy for library screening. In non-induced state, members of the library are similarly represented. Upon induction, most cells show decreased growth except those expressing non-toxic control genes (negative control). When library is induced in the presence of a small molecule that rescues toxicity of protein-of-interest-1, cells expressing protein-of-interest-1 show improved growth. If the barcoded library is induced in the presence of a non-specific small molecule, such as a drug that blocks our induction system, all barcoded cells including our toxic positive controls would show rescue (positive control and protein-of-interest-2).

FIG. 7: Growth of barcoded cell populations is highly reproducible. Growth in glucose, where toxic proteins are not expressed, results in barcode abundances that are highly correlated among replicates. Growth of barcoded cell populations in galactose, where toxic proteins are expressed, and barcode skewing occurs based on the toxicity of each protein expressed in each barcoded cell, still results in barcode abundances that are highly correlated.

FIG. 8: Spot assays of wild-type, Δpdr1 Δpdr3 and Δpdr1 Δpdr3 Δsnq2 yeast grown on yeast peptone dextrose (YPD) media in the presence of various small molecule compounds. Strains were grown to saturation in YPD media and serial dilutions (10-fold) of yeast were plated.

FIG. 9: Modulating the expression of multidrug exporters influences the level of rescue. Cells expressing pAG426-GAL-HIV vectors were transformed into either wild type (WT) or Δpdr1 Δpdr3 Δsnq2 (3Δ) yeast which show a reduced ability to export exogenous small molecules from the cell. Cells were then grown overnight in SC-ura glucose media and spotted at 10-fold serial dilution onto SC-ura galactose plates containing the indicated amount of HIV protease inhibitor (Lopinavir or Saquinavir). 3Δ cells mutant for multiple drug exporters showed significant increases in rescue compared to wild type cells, demonstrating increased penetrance of the compounds into the cell to mediate their effects.

FIG. 10: Results of small scale screen demonstrating the ability of DNA barcoded libraries in combination with various controls and modifications of the cellular chassis to obtain protein-drug interaction data including specificity characterization and dose-response information. Implementation of drug screening platform for proteases of viral or bacterial origin. Y axis represents drugs screened at 10 uM. X axis represents protein modeled and which cell line. WT=Natural expression of drug transporters. KO=No expression of PDR1, PDR3 and SNQ2. We detect significant enrichments for cells expressing HIV protease with known protease inhibitors of HIV with more sensitive detection in KO cells. Additionally, we detect a number of non-specific interactions in control-expressing cells.

FIG. 11: Serial 5-fold dilutions were made for each strain and then plated on selective galactose or glucose containing media and allowed to grow for 2 days before imaging. Expression of proteins implicated in neurodegenerative diseases cause toxicity when expressed in yeast.

FIG. 12A and FIG. 12B: NDD drug screen using our platform FIG. 12A: Implementation of drug screening platform for select neurodegenerative disease models identifies two protein-drug interactions as significant. Y axis represents drugs screened at 10 uM. X axis represents protein modeled and which cell line. WT=Natural expression of drug transporters. KO=No expression of multidrug transporters PDR1 and PDR3. FIG. 12B: Manual spot assay of cultures plated in consecutive 5-fold dilutions verifies significant protein-drug interaction for OPTN and Riluzole, note slight growth improvement and fact that TAF15 and mcherry expressing cells seem to show a decreased growth in the presence of Riluzole.

FIG. 13: DNA-barcoding strategy for library screening. Different DNA barcoded strains are shown as different shades. Under a non-induced state, members of the library were equally represented. Upon induction, most cells showed decreased growth except those expressing non-toxic control genes such as EYFP. When the library was induced in the presence of a small molecule that rescued toxicity of TDP-43, cells expressing TDP-43 showed improved growth. If the barcoded library was induced in the presence of a non-specific small molecule, such as a drug that blocked the induction system, all barcoded cells including the toxic positive controls would show rescue (DNAsel and FUS).

FIG. 14: DNA barcoding strategy for drug screening. Ten disease models were probed as a mixed pool across a set of 22 small molecules. For clarity only a portion of this experiment was shown. Of the >200 tested ALS gene-drug interactions, only two potential hits were found (shown). These hits, Riluzole and Celastrol, have been previously implicated in ALS or protein-aggregate biology. In these experiments cells containing HIV protease, DNAseI and mCherry served as controls. Of note, Darunavir is a known HIV protease inhibitor and shows the expected rescue of growth for cells expressing HIV protease.

FIG. 15: Off-target drug detection. Different DNA barcoded yeast strains were shown as different shades. TDP-43 and FUS represent inducible yeast models of ALS. Δrad52 and Δtub3 are barcoded strains which are designed to be highly sensitive to DNA damaging agents or mitotic inhibitors, respectively. When library members were induced in the presence of a non-toxic small molecule, cells expressing TDP-43 and FUS showed marked depletion. In contrast, if cells were induced in combination with a drug that caused DNA damage (MMS), Δrad52 cells, along with TDP-43 and FUS expressing cells, showed marked decline. By monitoring the abundance of each of the mutant yeast cell lines, the off-target effects of each tested compound were characterized.

FIG. 16: Drug dosing by modulating multidrug exporter expression. DNA barcoded yeast strains were shown as different shades. Strains differed in their expression of various multidrug transporters. When incubated with the same amount of drug, cells deleted for the major drug transporters (KO) accumulated more drug than wildtype (WT) cells or cells overexpressing the same exporters (OE).

FIG. 17: DNA-barcoding strategy for library screening. Different DNA barcoded strains were shown as different shades. Under a non-induced state, members of the library were equally represented (starting library). In the induced state most members exhibited decreased growth except those expressing non-toxic control genes such as EYFP (induced). When the library was induced in the presence of a small molecule that rescued toxicity of a specific kinase such as FYN, cells expressing FYN showed improved growth (induced+specific small molecule; FYN kinase). If the barcoded library was induced in the presence of a non-specific small molecule, such as a drug that blocked the induction system, all barcoded cells including the toxic positive controls would show rescue (induced+non-specific small molecule; HIV protease). The ratios represent proportion of each library member in the order presented in the legend (top-bottom).

FIG. 18: DNA barcoding strategy for drug screening. Ten ALS models were probed as a mixed pool across a set of 22 small molecules. For clarity, only a portion of this experiment was shown. Of the >200 tested ALS gene-drug interactions, two potential hits were found (shown). These hits, Riluzole and Celastrol, have been previously implicated in ALS or protein-aggregate biology. In these experiments, cells containing HIV protease and DNaseI served as positive controls, and cells containing mCherry served as negative controls. Of note, Darunavir is a known HIV protease inhibitor and shows the expected rescue of growth for cells expressing HIV protease.

FIG. 19: Off-target drug detection. DNA barcoded yeast strains were shown as different shades. FYN kinase and ZAP70 kinase represent yeast strains that, upon expression of these kinases, show growth suppression and are the targets against which inhibitors are to be identified. Δrad52 and Δtub3 are barcoded strains which are designed to be highly sensitive to DNA damaging agents or mitotic inhibitors, respectively. When library members were induced in the presence of a non-toxic small molecule, cells expressing FYN and ZAP70 showed marked depletion. In contrast, if cells were induced in combination with a drug that caused DNA damage (MMS), Δrad52 cells, along with FYN and ZAP70 expressing cells showed marked decline. The ratios represent the proportion of each library member in the order presented in the legend (top-bottom).

FIG. 20A-FIG. 20B: Modulating the expression of multidrug exporters enables several intracellular concentrations of drug to be sampled within a single assay, mimicking the effect of screening at different drug concentrations. FIG. 20A: To enable a pool of cells exposed to the same extracellular drug concentration to experience different intracellular concentrations of the compound, a set of yeast mutants were engineered overexpressing (OE) or knocked out (KO) for the major multidrug exporters within yeast: PDR5, YOR1, and SNQ2. Strains that differ in their expression of the multidrug transporters were shown as different shades. When incubated with the same amount of compound, cells depleted of the drug exporters (KO) accumulated more compound intracellularly than wild type (WT) cells or cells overexpressing the same exporters (OE). FIG. 20B: By altering drug exporter expression, the dose-response curve can be effectively shifted and by doing so capture compounds that would have been missed had only a single yeast chassis (KO, WT, or OE) been used in the screen. Dose-response curves for three different compounds (Drug A, B, and C) against a single disease model are illustrated. Concentration of compound added to the cells shown along the x-axis and response of the kinase expressing yeast cell as measured by cell growth shown on the y-axis. The dotted line represents the single concentration at which conventional drug screens are performed (e.g., 10 μM), with Drug A's effects only seen in the KO strain and Drug C's effect only being seen in the OE strain. In the absence of including additional yeast chassis, only Drug B would be seen as a hit. This assay tests all chassis (OE, WT and KO) at the same time within a single pool, mimicking the effect of screening a single chassis at multiple drug doses.

FIG. 21: Redundant barcoding increases assay sensitivity. A library containing up to 298 DNA barcoded neurodegenerative disease models and controls (x-axis) was tested as a pool against a set of 130 molecular chaperones of human and yeast origin (y-axis). For clarity, only a subset of these data are shown. As the number of barcodes (i.e., internal biological replicates) associated with each model are increased, the ability to identify genes that rescue the toxicity of expressing ALS-associated disease proteins increases. FDR=false discovery rate. All hits with FDR ≤0.15 were individually tested and found to be true rescuers.

FIG. 22: Final pool composition. Screening pool consists of various human kinase expressing yeast, controls, and off-target detectors. The numbers of barcodes occupied by each category are shown. Each of the kinases, controls, and off-target detectors are placed within each of the three drug transporter backgrounds. In addition, every combination of kinase, control, and off-target detector with drug transporter is represented by four independent strains, which are each uniquely barcoded further increasing the size of the screening pool.

FIG. 23A-FIG. 23C: Modulation of multidrug exporters enables the testing of multiple drug concentrations in parallel. FIG. 23A: Cells expressing wild type levels of multidrug exporters capture compound known to inhibit HIV protease activity. FIG. 23B: Similar to FIG. 23A, except cells are deficient in the expression of several multidrug exporters. A greater number of previously identified protease and kinase inhibitors are identified when using the drug exporter deficient background. FIG. 23C: The average degree of rescue, measured by the growth recovery of a specific yeast strain expressing a toxic protein, is higher in the knockout background as a result of higher intracellular drug concentrations. Of note all the data are from a single set of experiments and represent a subset of the 2880 potential protein-drug interactions that were tested. Furthermore, all of the hits shown are previously validated drug-target interactions. Each of the protein targets of interest expressed in the yeast cells is listed on the x-axis. The y-axis shows each of the compounds that were tested. FDR=false discovery rate, and is in relation to enrichment.

FIG. 24: Redundant barcoding increases the power to detect interactions in both mixed pool genetic and drug screening. A series of experiments were performed looking for genetic interactions that rescued the toxicity of aggregation prone proteins (left set of data) or small molecules that rescued the toxicity from expressing various proteases and kinases (right set of data). For each of the experiments models were examined with either 1, 2, 3, or 4 independent barcoded cell lines representing each model. An increase in assay sensitivity and capacity to detect weakly significant interactions occurs as each model is represented with additional independently barcoded lines within the mixed pool.

FIG. 25A-FIG. 25D: Wimpy Yeast allow for detection of drugs causing off-target toxicity in barcoded yeast pools. FIG. 25A: A number of drugs appeared to significantly rescue various yeast expressing different protease or kinases. FIG. 25B: When examining drugs that showed rescue of multiple models some showed marked depletion of various members of the wimpy yeast, suggesting that the molecules effects are through off-target mechanisms. FIG. 25C and FIG. 25D: For a subset of drugs with known specific rescue of various proteases and kinases, the present screen is able to capture these effects, and a corresponding decrease in growth of any of the tested wimpy yeast was not observed. For all figures each of the protein targets of interest expressed in the yeast cells or the genes knocked out of their genome is listed on the x-axis. The y-axis shows each of the compounds that were tested. FDR=false discovery rate, FDR for FIG. 25A and FIG. 25C are in relation to enrichment while FIG. 25B and FIG. 25D are in relation to depletion.

### DETAILED DESCRIPTION

The present disclosure relates, at least in part, to methods for high-throughput, multiplexed drug screening. The present methods are based on the expression of one or more proteins of interest, e.g., toxic proteins, in cells, e.g., a library comprising a plurality of cell types. These proteins, when induced, result in growth suppression, i.e., toxicity, in the cells, wherein the growth suppression is dependent on the protein's enzymatic or cellular function. Exposing the cells to a perturbation, e.g., an agent such as a drug candidate, which has the ability to alter the protein of interest's enzymatic or cellular function, can be detected as an improvement in the growth rate of the cells expressing the protein (e.g., rescue from the toxicity caused by the toxicity protein). By associating each protein of interest, e.g., each toxicity protein, with an associated unique DNA barcode within each cell type, changes in cellular fitness, e.g., toxicity, induced by the perturbation, can be determined via sequencing of the DNA barcodes, to thereby identify an effective drug candidate which is capable of inhibiting cellular toxicity. The methods of the disclosure provide that unique DNA barcodes can be assigned to many proteins of interest, and allow for many cell types expressing different target proteins to be pooled together and analyzed in the same experiment (e.g., concurrently), reducing inter-experimental variation and vastly increasing the information obtained from a single drug screen.

Moreover, by associating unique barcodes other cell types that serve a number of purposes, the methods of the present disclosure provide several additional advantages over current screening methods, allowing identification of important biological information about each tested agent. For example, the present disclosure provides methods to ascertain information regarding not only the effectiveness, but also the specificity, of a perturbation, e.g., a candidate agent such as a small molecule, to inhibit toxicity specifically related to the protein of interest.

In some aspects, the present disclosure also provides methods for identifying off-target effects of a particular candidate agent by producing highly sensitive cell types that are sensitive to a wide range of cellular insults, such as DNA-damage, and by tracking their fitness via their unique associated DNA barcodes, to thereby determine if a candidate agent has unintended off-target toxicities.

Furthermore, in another aspect, the present disclosure provides methods for simultaneously testing a range of candidate drug concentrations, e.g., within a single screen. For example, the disclosed methods provide for the determination of dose-response relationships within a single screen by generating several cell types that accumulate more or less of a candidate agent within the cell. In some embodiments, this is achieved by altering the expression (e.g., increasing, decreasing, or knocking out expression) of various drug importers and/or exporters on the cells to control the amount of candidate agent within the cell. Thus, although all of the cells are exposed to the same extracellular concentration of compound, different mutants that, for example, lack expression of certain drug exporters, will accumulate more drug intracellularly. By tracking the cells via their unique associated DNA barcodes, it is possible to determine a dose of a candidate agent having the desired effect on the cell, e.g., rescue of the toxicity phenotype.

In another aspect, by associating DNA barcodes with a number of control proteins, e.g., positive and/or negative control proteins, the present disclosure provides methods to detect non-specific drug interactions such as drugs that alter the genetic induction systems used to express the proteins of interest, e.g., toxicity proteins.

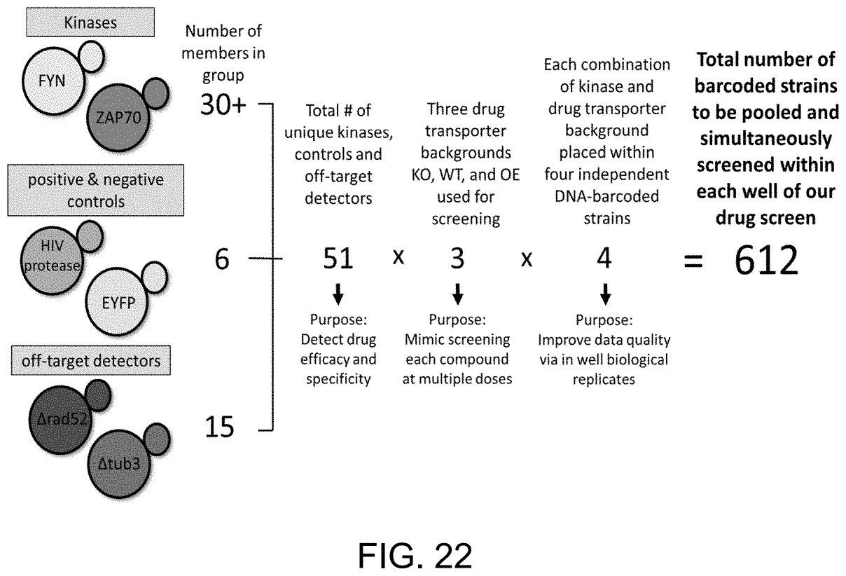

The present disclosure provides methods wherein, in some embodiments, a library is screened wherein the library contains multiple cell types, wherein each cell type comprises one of the following: an inducible target protein, e.g., toxicity protein, a positive control, or a negative control. The library can also contain cell types which have been modified to be off-target detectors (e.g., highly sensitive cell types). Each individual cell type contains its own unique associated barcode. The library can further comprise cell types having various drug transporter backgrounds, allowing for varying concentrations of the candidate agent within the cells. In some embodiments, the barcoded cell types can be pooled and simultaneously screened within a single well of a drug screen. (See, e.g., FIG. 22).

### Definitions

As used herein and in the appended claims, the singular forms “a”, “an”, and “the” include plural reference unless the context clearly dictates otherwise. Thus, for example, reference to a “peptide” is a reference to one or more peptides and equivalents thereof known to those skilled in the art, and so forth.

As used herein, the term “about” means plus or minus 20% of the numerical value of the number with which it is being used. Therefore, about 50% means in the range of 40%-60%.

The term “amplification” as used herein refers to a replication of genetic material that results in an increase in the number of copies of that genetic material.

The term “barcode” or “DNA barcode” as used herein, refers to any unique, non-naturally occurring, nucleic acid sequence that may be used to identify the originating source of a nucleic acid fragment. A barcode can be associated with a particular cell type, e.g., a proliferating cell type, wherein the cell type comprises a unique barcode. A barcode can be inserted within in the genome of the cell or can be carried on a plasmid within the cell. For example, a barcode can be associated with a cell type comprising a particular characteristic or a cell type expressing a particular protein, e.g., a toxicity protein. In some embodiments, cell types comprising a certain cell surface drug transporter are barcoded with the same barcode. In some embodiments, cell types comprising a certain characteristic or protein, for example, a toxicity protein, a positive or negative control, a drug transporter background, or a highly sensitive cell type, or specific combinations thereof, are barcoded with the same barcode. In other embodiments, cell types comprising a negative control non-toxic inducible protein, e.g., a non-toxic control protein, are barcoded with the same barcode. In other embodiments, cell types comprising a positive control protein, e.g., a positive control inducible toxicity protein, are barcoded with the same barcode. Barcoding may be performed based on any of the methods disclosed in, for example, patent publication WO 2014047561 A1, “Compositions and methods for labeling of agents,” Birrell et al., (2001) Proc. Natl Acad. Sci. USA 98, 12608-12613; Giaever, et al., (2002) Nature 418, 387-391; Winzeler et al., (1999) Science 285, 901-906; and Xu et al., (2009) Proc Natl Acad Sci USA. February 17; 106(7):2289-94); Rosenberg et al. (2018) Science, 360; 176-182; Quinodoz, et al. (2018) Cell, 174, 744-757, the contents of each of which are hereby incorporated herein by reference.

The transitional term “comprising”, which is synonymous with “including,” “containing,” or “characterized by,” is inclusive or open-ended and does not exclude additional, unrecited elements or method steps. See MPEP 2111.03.

As used herein, the term “toxicity protein,” refers to any protein which functions to cause toxicity (either directly or indirectly) in a cell in which it is expressed, wherein the toxicity is dependent on the protein's catalytic activity. In some embodiments, toxicity refers to a decrease, or inhibition of cell growth rate, cell division, or proliferation, as compared to a control cell that does not express the toxicity protein. A toxicity protein can be encoded by a gene which is exogenous to the cell in which it is expressed (a toxicity gene). Induction of expression of the toxicity gene encoding the toxicity protein in the cell results in a decrease or inhibition cell growth rate, cell division, or proliferation of a cell.

As used herein, the terms “next generation sequencing” or “NGS” refers to a high-throughput method used to determine a barcodes identifying complex formation. This technique utilizes DNA sequencing technologies that are capable of processing multiple DNA sequences in parallel.

As used herein, the term “oligonucleotide” refers to a nucleic acid such as deoxyribonucleic acid (DNA), ribonucleic acid (RNA), or DNA/RNA hybrids and includes analogs of either DNA or RNA made from nucleotide analogs known in the art (see, e.g. U.S. Patent or Patent Application Publications: U.S. Pat. Nos. 7,399,845, 7,741,457, 8,022,193, 7,569,686, 7,335,765, 7,314,923, 7,335,765, and 7,816,333, US 20110009471, the entire contents of each of which are incorporated herein by reference). Oligonucleotides may be single-stranded (such as sense or antisense oligonucleotides), double-stranded, or partially single-stranded and partially double-stranded.

The term “off-target” and the phrase “off-target effects” refer to any instance in which a perturbation, e.g., an agent such as a small molecule or other drug, directed against a given target protein, e.g., toxicity protein, causes an undesirable, or unintended phenotypic effect by interacting either directly or indirectly with a mRNA sequence, a DNA sequence or a cellular protein or any other moiety that is distinct from the toxicity protein induced in the cell. Off-target toxicity may result in loss of desirable function, gain of non-desirable function, or even death at the cellular level. Off-target effects, e.g., toxicity, may arise via an entirely different mechanism from the mechanism resulting in the targeted effect of the a perturbation, e.g., an agent such as a small molecule or other drug, being screened.

As used herein, the term “perturbation” or “perturbation event”, is understood to mean one or more agents (e.g., chemical entities, pharmaceuticals, and drugs) or events (e.g., environmental perturbations or genetic perturbations) are capable of interacting with a target protein, e.g., a toxicity protein, either directly or indirectly, to modulate, e.g., reduce, inhibit or ameliorate, a cellular phenotype, e.g., cellular toxicity, over a period of time through exposure or contact with one or more parts of the cell. Amelioration of toxicity can be measured by observing a restoration of cell division. In one embodiment, the agent is capable of diffusing through the cell membrane, or of having an effect inside the cell. In another embodiment, the agent is a soluble agent. A perturbation can be a single agent or event, or a mixture of agents, or one or more events and agents in combination, including a mixture in which not all constituents are identified or characterized.

With respect to perturbations that are agents, the chemical and physical properties of an agent or its constituents may not be fully characterized. An agent can be defined by its structure, its constituents, or a source that under certain defined conditions produces the agent. An example of an agent is a heterogeneous substance, that is a molecule or an entity that is not present in or derived from the biological system, and any intermediates or metabolites produced therefrom after contacting the biological system. Non-limiting examples of agents include chemical entities, pharmaceuticals, and drugs such as small molecules, biologics such as antibodies, proteins, peptides, enzymes, lipids, carbohydrates, nucleic acids (e.g., siRNAs, antisense oligonucleotides, shRNA, or miRNA), aptamers, or alkaloids. Additional non-limiting examples of agents include, but are not limited to nutrients, metabolic wastes, poisons, narcotics, toxins, therapeutic compounds, stimulants, relaxants, natural products, manufactured products, food substances, pathogens (prion, virus, bacteria, fungi, protozoa), vitamins, metals, heavy metals, minerals, oxygen, ions, hormones, neurotransmitters, inorganic chemical compounds, organic chemical compounds, particles or entities whose dimensions are in or below the micrometer range, by-products of the foregoing and mixtures of the foregoing.

Examples of environmental perturbations include, but are not limited to, microorganisms, environmental conditions, environmental forces, physical forces, radiation, electromagnetic waves (including sunlight), increase or decrease in temperature, shear force, fluid pressure, electrical discharge(s) or a sequence thereof, or trauma. Examples of genetic perturbations include, but are not limited to, genetic editing or regulation, gene activation or gene inhibition.

Some agents may not perturb a biological system unless it is present at a threshold concentration or it is in contact with the biological system for a period of time, or a combination of both. Exposure or contact of an agent resulting in a perturbation may be quantified in terms of dosage. Thus, a perturbation can result from a long-term exposure to an agent. The period of exposure can be expressed by units of time, by frequency of exposure, or by the percentage of time within the actual or estimated life span of the subject. A perturbation can also be caused by withholding an agent from or limiting supply of an agent to one or more parts of a biological system.

A “promoter”, as used herein, refers to an array of nucleic acid sequences that direct transcription of a nucleic acid. As used herein, a promoter includes necessary nucleic acid sequences near the start site of transcription. A promoter also optionally includes distal enhancer or repressor elements, which can be located as much as several thousand base pairs from the start site of transcription.

An “inducible promoter”, as used herein, is a promoter that is active under environmental or developmental regulation. Examples of inducible promoters are known in the art and described herein. See, e.g., Armakola M, et al., Nat Genet 2012; 44:1302-9; Jovicid A, et al., Nat Neurosci 2015; 18:1226-9; Sun Z, et al., PLoS Biol 2011; 9:e1000614; Yan Z, et al., Nat Methods 2008; 5:719-25, the contents of which are hereby incorporated herein by reference.

The term “operably linked”, as used herein, refers to a functional linkage between a nucleic acid expression control sequence (such as a promoter) and a second nucleic acid sequence, wherein the expression control sequence directs transcription of the nucleic acid corresponding to the second sequence.

The terms “test agent,” “test compound,” and “candidate agent”, used interchangeably herein, refer to any chemical entity, pharmaceutical, drug, and the like that is a candidate for use as a drug. In some embodiments, the test compound is a candidate for use to alter (e.g., enhance or inhibit) the activity of a toxicity protein. In some embodiments, the test compound may modulate the genomic state of a cell, e.g., the transcriptomic, genetic, and/or epigenetic state of the cell (e.g., the modulation of which is characterized using the methods of the present disclosure). Test compounds comprise both known and potential therapeutic compounds. A test compound can be determined to be therapeutic by screening using the screening methods of the present disclosure. Examples of test compounds include, but are not limited to, small molecules, carbohydrates, monosaccharides, oligosaccharides, polysaccharides, amino acids, peptides, oligopeptides, polypeptides, proteins, nucleosides, nucleotides, oligonucleotides, polynucleotides, including DNA and DNA fragments, RNA and RNA fragments and the like, lipids, retinoids, steroids, prodrugs, antibodies or portions thereof (e.g., antibody fragments), glycopeptides, glycoproteins, proteoglycans and the like, and synthetic analogues or derivatives thereof, including peptidomimetics and the like, and combinations thereof. A test compound can be determined to be capable of altering the activity of a toxicity protein in a cell using a method of the present disclosure. Test compounds comprise both known and potential therapeutic compounds. A test compound can be determined to be therapeutic by screening using the screening methods of the present disclosure.

The term “small molecule” refers to organic compounds generally having a molecular weight less than about 1000, preferably less than about 500, which are prepared by synthetic organic techniques, such as by combinatorial chemistry techniques.

As used herein, the term “drug” refers to a pharmacologically active molecule that is used to diagnose, treat, or prevent diseases or pathological conditions in a physiological system (e.g., a subject, or in vivo, in vitro, or ex vivo cells, tissues, and organs).

Methods of the Disclosure

The present disclosure is based, at least in part, on the discovery of methods, including multiplexed, high-throughput methods of screening for an agent capable of interacting with a target protein, e.g., a toxicity protein, either directly or indirectly, to modulate, e.g., reduce, inhibit or ameliorate, a cellular phenotype, e.g., cellular toxicity, over a period of cell proliferation through exposure or contact with one or more parts of the cell, by measuring DNA barcodes uniquely associated with the toxicity protein, as compared to a control.

In one aspect, the present disclosure provides methods of screening for an agent, e.g., a drug such as a small molecule, capable of specifically inhibiting a toxicity protein in a cell, the method comprising the steps of (a) providing a library comprising a plurality of proliferating cell types in the presence or absence of the agent, wherein each cell type comprises one or more inducible toxicity protein, wherein each proliferating cell type comprises a unique associated barcode, and wherein the library further comprises one or more control cell types selected from: (1) a proliferating cell type comprising a negative control inducible non-toxic protein, (2) a proliferating cell type comprising a positive control inducible toxicity protein, and (3) one or more proliferating cell types highly sensitive to cellular insults, wherein each control cell type comprises a unique associated control barcode; (b) inducing the expression of each toxicity protein and the one or more control proteins; (c) determining the relative number of unique associated barcodes and the unique associated control barcodes after a period of cell proliferation; and (d) comparing the relative number of unique associated barcodes to one or more of the unique associated control barcodes, to thereby determine the effectiveness and specificity of the agent, e.g., a drug such as a small molecule, on the one or more toxicity proteins. In some embodiments, all of the steps are performed in a single experiment, e.g., concurrently.

The term “cell type” may be used interchangeably with “strain.” According to one aspect, the methods employ greater than 5 cell types or strains, greater than 10 cell types or strains, greater than 20 cell types or strains, greater than 30 cell types or strains, greater than 40 cell types or strains, greater than 50 cell types or strains, greater than 60 cell types or strains, greater than 70 cell types or strains, greater than 80 cell types or strains, greater than 90 cell types or strains, greater than 100 cell types or strains, greater than 150 cell types or strains, greater than 200 cell types or strains, greater than 300 cell types or strains, greater than 400 cell types or strains, greater than 500 cell types or strains, greater than 600 cell types or strains, greater than 700 cell types or strains, greater than 800 cell types or strains, greater than 900 cell types or strains, greater than 1000 cell types or strains, greater than 2000 cell types or strains, greater than 3000 cell types or strains, and the like. In some embodiments, each cell type or strain including a particular exogenous protein includes a barcode unique to the cell type or strain. In some embodiments, each cell type or strain including a particular toxicity protein exhibits a lowered or slowed rate of cell growth or proliferation, or lower absolute number of cells, compared to the cell type or strain without the toxicity protein. Determining the number of barcodes within a population of cells, such as by DNA sequencing, is representative of the number of cells in a cell type or strain in the population. In this manner, the number of barcodes is representative of the number of cells in a cell type or strain and an increase or decrease in the number of barcodes is an indication of whether a particular perturbation has inhibited or has activated or has had no effect on the exogenous protein. According to one embodiment, a plurality of cell types or strains in a mixture, each having a different toxicity protein and associated growth rate, may be subjected to a particular perturbation and then the growth rates or amount of cells within a cell type or strain may be determined by sequencing the population of cells within the mixture to determine the number of barcodes. The number of barcodes indicates the number of cells of a cell type or strain present after a perturbation period and, therefore, indicates whether the toxicity or growth rate of a cell type with a particular exogenous protein increased, or decreased or stayed the same in response to the particular perturbation. In this manner, many different cell types or strains with exogenous toxicity proteins may be assayed in response to a perturbation, e.g., an agent such as a drug.

In some embodiments, the methods of the disclosure comprise testing more than one toxicity protein against one or more perturbations, e.g., agents, in the same screen. For example, 1, 5, 10, 15, 20, 25, 30, 35, 40, 45, 50, 55, 60, 65, 70, 75, 80, 85, 90, 100, 200 or more toxicity proteins can be included in a plurality of cell types, wherein each unique cell type comprises a unique associated barcode.

In some embodiments, each proliferating cell type further comprises one or more cell surface drug transporter having varying levels of expression for identifying an effective dosage of the agent having the ability to inhibit cellular toxicity. In some embodiments, each combination of an inducible toxicity protein and a level of expression of a cell surface drug transporter is associated with a unique barcode within the cell type comprising said combination.

In some embodiments, the method further comprises (e), which includes pooling results of step (d) from the cell types comprising the same combination of the inducible toxicity protein and the level of expression of the cell surface drug transporter.

A negative control inducible non-toxic protein can comprise any inducible protein that has no toxic effect on the cell type in which it is expressed. A cell type comprising the negative control inducible non-toxic protein comprises a unique associated control barcode and, thus, can serve as a control, or reference, for barcode depletion when induced. Inducible non-toxic proteins that can be used in the methods of the disclosure include, are known in the art, and include, but are not limited to enhanced yellow fluorescent protein EYFP, green fluorescent protein (GFP), and similar non-toxic inducible proteins (see, e.g., Snapp, Trends Cell Biol. 2009 Nov.; 19(11): 649-655).

A positive control inducible toxicity protein can comprise any inducible protein that has toxic effects on the cell type in which it is expressed, but through mechanisms that are orthogonal to the class of toxicity proteins being screened. A cell type comprising a positive control inducible toxicity protein comprises a unique associated control barcode. Thus, positive control inducible toxicity proteins can serve to identify perturbations, e.g., agents such as small molecules, that have non-specific effects, such as modulating or interfering with the genetic systems used to induce toxic protein expression.

Detection of Off-Target Effects

To detect drugs that have additional pleotropic effects, which may confound the identification of agents which inhibit toxicity induced by the target toxicity protein by appearing to rescue specific models, but in fact is a result of off-target effects rather than on-target enzymatic inhibition, control cell types that are highly sensitive to cellular insults can be included in the screening methods of the disclosure.

Cell types that are highly sensitive to cellular insults can comprise proliferating cell types that are highly sensitive to a wide variety of off-target drug toxicities. For example, highly sensitive cell type comprise cell types that are highly sensitive to perturbations to various biological pathways other than the toxicity protein pathway being targeted, or are highly sensitive to DNA damaging agents or mitotic inhibitors. In some embodiments, highly sensitive cell types can be engineered using genetic mutation to selectively render the cells hypersensitive to perturbations to various core cellular pathways. In some embodiments, the highly sensitive cell type is a cell that carries RAD gene mutations or deletions. In other embodiments, the highly sensitive cell type contains a mutation in a gene such as LEA1, CHO2, RFM1, LSM1, HOC1, ROM2, HAC1, SMY1, ABP1, ERV14, SNT1, PFA4, SSD1, GSF2, and/or CLB2. For example, “wimpy” yeast can be utilized that are engineered to be sensitive to a wide variety of off-target drug toxicities (i.e., perturbations to various biological pathways). See, for example, Piotrowski J S, et al., Nat Chem Biol. 2017; 13(9):982-993, the contents of which are hereby incorporated herein by reference, and Example 4, below).

Some non-specific candidate agents can create false positives in a screening assay through off-target effects resulting in general alteration of cell growth. By including highly sensitive cells types in the screening methods of the disclosure, which comprise unique associated barcodes, candidate agents causing off-target effects can be easily identified and ruled out. In contrast to non-specific candidate agents, specific agents whose mechanism of action directly affects the cell types comprising the target toxicity protein, but does not affect the highly sensitive cells, can be identified.

Protein-Drug Interaction Detection with Multi-Dose Testing

Screening at a single concentration prevents the identification of drugs with therapeutic effects outside of the tested dosage or with effects that change at different drug concentrations. However, the present invention provides methods for screening an agent, e.g., a small molecules, at multiple concentrations within the cells, when exposed to a single concentration of the agent, e.g., while simultaneously screening for the effect of the agent on the target protein to reduce toxicity.

By manipulating the expression of drug exporters or introducing into cells an additional drug importer or exporter, the amount of compound within each of the cells can be regulated. As DNA-barcoding allows simultaneous testing of multiple yeast models at a time, by placing the same target protein within a background lacking drug exporters or containing normal levels of these exporters, how a given compound interacts with the target protein of interest when present at elevated or normal, intracellular concentrations, respectively, can be investigated. Accordingly, in some embodiments, the present disclosure provides methods for screening agents at multiple cellular concentrations by utilizing cell types that overexpress, underexpress, or lack one or more drug transporter, e.g., a cell surface multidrug transporter.

In some embodiments, the cell surface drug transporter is a drug exporter. In some embodiments, the cell surface drug transporter is a drug importer. For example, yeast strains can be engineered to either overexpress or lack one or more of the three main multi-drug exporters (PDR1, PDR3, PDR5, YOR1, SNQ2) within S. cerevisiae, rendering these modified strains either extremely adept or inefficient at pumping out small molecules, respectively. In some embodiments, the same target protein(s) can be placed into the cell types comprising the various modified drug transporters. This approach gives some cell lines in the mixed pool a low intracellular drug concentration while other cell lines in the mixed pool receive a high intracellular drug concentration when exposed to the same extracellular drug concentration. Each cell line can then be DNA barcoded and pooled, and grown in the presence of a candidate agent at a single concentration. Agents can be identified that are more likely to show rescue of cellular toxicity in backgrounds comprising one modified drug transporter versus another. For example, cells that are deficient in drug exporters, leading to a higher intracellular concentrations of drug, may be more likely to show rescue with compounds known to inhibit each of the target toxicity proteins.

Using the information obtained from the screening methods described herein, lead compounds are identified such as those that show no sign of toxicity at higher intracellular doses (i.e., within strains that lack exporter expression) and maintain activity even at lower intracellular doses (i.e., within strains that overexpress the major drug exporters).

Accordingly, in some aspects, the present disclosure provides methods of identifying an effective dosage of a drug having the ability to inhibit cellular toxicity in a cell, comprising (a) providing a library comprising a plurality of proliferating cell types, wherein each cell type comprises one or more inducible toxicity protein and having varying levels of expression of one or more cell surface drug transporter, and wherein each proliferating cell type comprises a unique associated barcode, and wherein the library further comprises one or more control cell types selected from: (1) a proliferating cell type comprising a negative control inducible non-toxic protein, and (2) a proliferating cell type comprising a positive control inducible toxicity protein, wherein each control cell type comprises a unique associated control barcode; (b) inducing the expression of each toxicity protein in the presence or absence of a single concentration of the drug, wherein the proliferating cell types contain different intracellular concentrations of the drug as a result of the varying levels of expression of one or more cell surface drug transporter; (c) determining the relative number of unique associated barcodes after a period of cell proliferation; and (d) comparing the relative number of unique associated barcodes to one or more controls, to thereby determine the effective intracellular concentration of the drug on the one or more proliferating cell types.

In some embodiments, the method further comprises step (e), which is pooling results of step (d) from the cell types comprising the same combination of the inducible toxicity protein and the level of expression of the cell surface drug transporter.

Use of Redundant DNA Barcodes

In some embodiments, as a further method to ensure quality of data ascertained from the screening methods of the present disclosure, redundant DNA barcodes may be utilized. Technical variation (e.g., library bias introduced during PCR amplification of DNA barcodes) and biological variation (e.g., cells accumulating suppressor mutations), can be considered when performing the screening methods on a large scale. To help mitigate the potential effects of technical and biological variation, a redundant barcoding approach can be used. In these embodiments, each of the combinations between drug transporter background and toxicity protein/control(s)/off-target detectors are placed into four different uniquely barcoded strains. By looking at the collective behavior of all four independently barcoded cells associated with the same genotype (e.g., FYN kinase expression, with drug exporters knocked out), technical and biological variations are under improved control, as it is unlikely that by chance all four barcoded cell lines would exhibit the same technical or biological deviation.

Exemplary Proliferating Cell Types

Proliferating cell types or organisms useful in the methods described herein are those which proliferate at a rate sufficient to carry out experiments in a desirable period of time. The proliferating cell types or organisms are also capable of expressing a phenotype associated with an exogenous protein, such as a toxicity protein. According to one aspect, exemplary proliferating cell types are capable of exhibiting a lowering of cell growth in response to the presence of an endogenous protein that is toxic to the cell type. The proliferating cell type or organism may be genetically altered to include the toxicity gene which is expressed by the cell or otherwise induced to be expressed to produce the toxicity protein.

Exemplary proliferating cell types include eukaryotic cells or prokaryotic cells. Exemplary eukaryotic cells include yeast cells, fungal cells, insect cells, and mammalian cells.

Exemplary eukaryotic cells include yeast strains or fungus strains. Exemplary yeast strains include, but are not limited to, Saccharomyces cerevisia (and subtypes such as S288C, CEN.PK etc), genus Saccharomyces (e.g., S. cerevisiae, S. bayanus, S. boulardii, S. pastorianus, S. rouxii and S. uvarum), Schizosaccharomyces (e.g., S. pombe), Kluveromyces (e.g., K. lactis and K. fragilis), genus Candida (C. albicans, C. krusei and C. tropicalis) and Pichia pastoris, and the like. Yeast strains present a preferred exemplary cell type for use in the methods described herein, as yeast have tractable systems for genetic modification and are relatively inexpensive to maintain compared to other cellular models. However, the screening methods of the disclosure are not confined to yeast and are readily adaptable to work in any cellular system.

Exemplary fungus strains include Aspergillus nidulans, A. oryza, A. niger, A. sojae, and the like.

Exemplary eukaryotic cells include mammalian cells. Exemplary mammalian cells include, but are not limited to, HEK 293, Chinese Hamster Ovary cells, HEK 293F, HEK 293H, HEK 293A, HEK 293FT, HEK293T, CHO DG44, CHO—S, CHO-DXB11, Expi293F, ExpiCHO-S, T-Rex, Hela, MCF7, COST, NIH 3T3, U20S, A375, A549, N2A, PGP1 iPS, BHK, Hap1, Jurkat, NO, and the like.

Exemplary prokaryotic cells include bacteria strains. Exemplary bacterial strains include, but are not limited to, E. coli, B. subtilis, S. aureus, S. typhi, M genitalium, V cholera, P. putida and the like. Exemplary cell that can be used in the methods of the disclosure are described in, for example, US Patent Application Publication No. US2019/0161751.

Exemplary Exogenous Genes and Proteins

Toxicity proteins used in the screening methods of the present disclosure are not limited to any particular class of proteins. Toxicity proteins include any protein which functions to cause toxicity to a cell in which it is expressed, wherein the toxicity is dependent on the protein's catalytic activity.

Exemplary toxicity proteins that can be used in the methods of the disclosure include, but are not limited to, kinases, proteases, aggregation-prone proteins, viral integrases, nucleic acid binding proteins, structural proteins, protein chaperones, phosphatases, small GTPases, ubiquitin ligases DNA or RNA polymerases, caspases, hydrolases, ligases, oxidoreductases, transcription factors, cell adhesion molecules, cell junction molecules, isomerases, transferases, adapter proteins, or reverse transcriptases.

Other exemplary toxicity proteins include disease-associated toxicity proteins, e.g., toxicity proteins which are responsible for or otherwise involved in or related to a disease or disorder, or symptoms associated with a disease or disorder. Exemplary disease-associated toxicity proteins include, but are not limited to, neurodegenerative disease-associated protein such as amyotrophic lateral sclerosis (ALS)-associated proteins, Parkinson's disease-associated proteins, and Alzheimer's disease-associated proteins. Additional examples of disease-associated toxicity proteins include, but are not limited to, Abeta, Androgen receptor (AR), a-syn A30P, a-syn A53T, a-syn WT, ataxin1, Ataxin1 [Q84], ataxin3, ATXN7, C9orf72 GA100, C9orf72 GA200, C9orf72 GA50, C9orf72 GR100, C9orf72 GR50, C9orf72 PR50, CHOPS M8, CHOPS Wt, EWSR1, EWSR1c1655t, EWSRlg1532c, EWSR1g1750a, FUS WT, FUS-P525L, hnRNPA1 WT, hnRNPA1D262V, hnRNPA2B1 D290V, hnRNPA2B1WT, htt72Q, htt103Q, Htt46Q, PABPN1, SOD1 A4V, SOD1 G85R, SOD1 G93A, SOD1 WT, TAF15, TAF15c1222t, TAF15g1172a, Tau, TDP43, TDP-43 G294A, TDP-43 M337V, TDP-43 Q331K, UBQLN2, CHMP2B, PABPN1, ARX, SOX3, RUNX2, ZIC2, PHOX2B, HOXD13, HOXA13, FOXL2, ATXN2, CACNA1A, PrP, and TBP.

Exemplary Methods of Modifying a Cell Type with an Exogenous Nucleic Acid

Cell types according to the present disclosure may be modified to include one or more exogenous nucleic acids (e.g., one or more exogenous toxicity genes), which are expressed by the cell to produce one or more exogenous protein (e.g., one or more exogenous toxicity protein).

As an example, yeast cells may be genetically modified using methods known to those of skill in the art including by LiAc, Electroporation, Biolistic transformation as described in Kawai S, Hashimoto W, Murata K. Transformation of Saccharomyces cerevisiae and other fungi: Methods and possible underlying mechanism. Bioengineered Bugs. 2010; 1(6):395-403 hereby incorporated by reference in its entirety.

Amplification Methods

Nucleic acids within cells of a pool of proliferating cells, such as yeast, bacteria or mammalian cells, may be amplified using methods known to those of skill in the art. Exemplary amplification methods include contacting a nucleic acid with one or more primers that specifically hybridize to the nucleic acid under conditions that facilitate hybridization and chain extension. Exemplary methods for amplifying nucleic acids include the polymerase chain reaction (PCR) (see, e.g., Mullis et al. (1986) Cold Spring Haab. Symp. Quant. Biol. 51 Pt 1:263 and Cleary et al. (2004) Nature Methods 1:241; and U.S. Pat. Nos. 4,683,195 and 4,683,202), anchor PCR, RACE PCR, ligation chain reaction (LCR) (see, e.g., Landegran et al. (1988) Science 241:1077-1080; and Nakazawa et al. (1994) Proc. Natl. Acad. Sci. U.S.A. 91:360-364), self-sustained sequence replication (Guatelli et al. (1990) Proc. Natl. Acad. Sci. U.S.A. 87:1874), transcriptional amplification system (Kwoh et al. (1989) Proc. Natl. Acad. Sci. U.S.A. 86:1173), Q-Beta Replicase (Lizardi et al. (1988) BioTechnology 6:1197), recursive PCR (Jaffe et al. (2000) J. Biol. Chem. 275:2619; and Williams et al. (2002) J. Biol. Chem. 277:7790), the amplification methods described in U.S. Pat. Nos. 6,391,544, 6,365,375, 6,294,323, 6,261,797, 6,124,090 and 5,612,199, isothermal amplification (e.g., rolling circle amplification (RCA), hyperbranched rolling circle amplification (HRCA), strand displacement amplification (SDA), helicase-dependent amplification (HDA), PWGA) or any other nucleic acid amplification method using techniques well known to those of skill in the art.

Barcoding Methods

In some embodiments, the methods of the disclosure comprise barcoding various cell types, e.g., cell types comprising a target inducible protein of interest, or other measurable phenotype or background, and/or control, with unique associated DNA barcodes, as described herein.

In instances where barcode identification and/or quantification is performed by sequencing, including e.g., next generation sequencing methods, conventional considerations for barcodes detected by sequencing will be applied. In some instances, commercially available barcodes and/or kits containing barcodes and/or barcode adapters may be used or modified for use in the methods described herein, including e.g., those barcodes and/or barcode adapter kits commercially available from suppliers such as but not limited to, e.g., New England Biolabs (Ipswich, Mass.), Illumina, Inc. (Hayward, Calif.), Life Technologies, Inc. (Grand Island, N.Y.), Bio Scientific Corporation (Austin, Tex.), and the like, or may be custom manufactured, e.g., as available from e.g., Integrated DNA Technologies, Inc. (Coralville, Iowa).

In some embodiments, barcodes used in the methods of the disclosure can include indexed PCR primers, e.g., Tru-seq, or AmpliSeq, Nextera PCT primers. For example, both the forward and reverse primer of the PCR reaction can contain an 8 bp barcode, the pairwise combinations of 8 forward primers and 12 reverse primers would generate an additional 96 barcodes that can effectively be seen as the final step of a split-pool barcoding strategy, as done in the Split-SEQ paper (Rosenberg et al. 2018, Science Vol. 360, Issue 6385, pp. 176-182). Indexed adapters provide another round of barcoding. In particular, the combination of indexes used for PCR provide additional complex barcode complexity.

As used herein, the term “barcode” refers to an oligonucleotide sequence that allows a corresponding nucleic acid sequence (e.g., an oligonucleotide fragment) to be identified, retrieved and/or amplified. In certain embodiments, barcodes can each have a length within a range of from 4 to 36 nucleotides, or from 6 to 30 nucleotides, or from 8 to 20 nucleotides. In certain exemplary embodiments, a barcode has a length of 4 nucleotides. In certain aspects, the melting temperatures of barcodes within a set are within 10° C. of one another, within 5° C. of one another, or within 2° C. of one another. In other aspects, barcodes are members of a minimally cross-hybridizing set. That is, the nucleotide sequence of each member of such a set is sufficiently different from that of every other member of the set that no member can form a stable duplex with the complement of any other member under stringent hybridization conditions. In one aspect, the nucleotide sequence of each member of a minimally cross-hybridizing set differs from those of every other member by at least two nucleotides. Barcode technologies are known in the art and are described in Winzeler et al. (1999) Science 285:901; Brenner (2000) Genome Biol. 1:1 Kumar et al. (2001) Nature Rev. 2:302; Giaever et al. (2004) Proc. Natl. Acad. Sci. USA 101:793; Eason et al. (2004) Proc. Natl. Acad. Sci. USA 101:11046; and Brenner (2004) Genome Biol. 5:240.

Sequencing Methods

Nucleic acids within cells of a pool of proliferating cells, such as yeast or bacteria or mammalian cells, may be sequenced using methods known to those of skill in the art such as high throughput disclosed in Mitra (1999) Nucleic Acids Res. 27(24):e34; pp. 1-6.

In certain embodiments, methods of determining the sequence of one or more nucleic acid sequences of interest, e.g., polynucleotides, oligonucleotides and/or oligonucleotide fragments, are provided. Determination of the sequence of a nucleic acid sequence of interest can be performed using variety of sequencing methods known in the art including, but not limited to, sequencing by hybridization (SBH), sequencing by ligation (SBL), quantitative incremental fluorescent nucleotide addition sequencing (QIFNAS), stepwise ligation and cleavage, fluorescence resonance energy transfer (FRET), molecular beacons, TaqMan reporter probe digestion, pyrosequencing, fluorescent in situ sequencing (FISSEQ), FISSEQ beads (U.S. Pat. No. 7,425,431), wobble sequencing (PCT/US05/27695), multiplex sequencing (U.S. Ser. No. 12/027,039, filed Feb. 6, 2008; Porreca et al (2007) Nat. Methods 4:931), polymerized colony (POLONY) sequencing (U.S. Pat. Nos. 6,432,360, 6,485,944 and 6,511,803, and PCT/US05/06425); nanogrid rolling circle sequencing (ROLONY) (U.S. Ser. No. 12/120,541, filed May 14, 2008), allele-specific oligo ligation assays (e.g., oligo ligation assay (OLA), single template molecule OLA using a ligated linear probe and a rolling circle amplification (RCA) readout, ligated padlock probes, and/or single template molecule OLA using a ligated circular padlock probe and a rolling circle amplification (RCA) readout) and the like. High-throughput sequencing methods, e.g., on cyclic array sequencing using platforms such as Roche 454, Illumina Solexa, AB-SOLiD, Helicos, Polonator platforms and the like, can also be utilized. High-throughput sequencing methods are described in U.S. Ser. No. 61/162,913, filed Mar. 24, 2009. A variety of light-based sequencing technologies are known in the art (Landegren et al. (1998) Genome Res. 8:769-76; Kwok (2000) Pharmocogenomics 1:95-100; and Shi (2001) Clin.

Additional sequencing methods useful in the present disclosure include Shendure et al., Accurate multiplex polony sequencing of an evolved bacterial genome, Science, vol. 309, p. 1728-32. 2005; Drmanac et al., Human genome sequencing using unchained base reads on self-assembling DNA nanoarrays, Science, vol. 327, p. 78-81. 2009; McKernan et al., Sequence and structural variation in a human genome uncovered by short-read, massively parallel ligation sequencing using two-base encoding, Genome Res., vol. 19, p. 1527-41. 2009; Rodrigue et al., Unlocking short read sequencing for metagenomics, PLoS One, vol. 28, e11840. 2010; Rothberg et al., An integrated semiconductor device enabling non-optical genome sequencing, Nature, vol. 475, p. 348-352. 2011; Margulies et al., Genome sequencing in microfabricated high-density picolitre reactors, Nature, vol. 437, p. 376-380. 2005; Rasko et al. Origins of the E. coli strain causing an outbreak of hemolytic-uremic syndrome in Germany, N. Engl. J. Med., Epub. 2011; Huffer et al., Labeled nucleoside triphosphates with reversibly terminating aminoalkoxyl groups, Nucleos. Nucleot. Nucl., vol. 92, p. 879-895. 2010; Seo et al., Four-color DNA sequencing by synthesis on a chip using photocleavable fluorescent nucleotides, Proc. Natl. Acad. Sci. USA., Vol. 102, P. 5926-5931 (2005); Olejnik et al.; Photocleavable biotin derivatives: a versatile approach for the isolation of biomolecules, Proc. Natl. Acad. Sci. USA., vol. 92, p. 7590-7594. 1995; US 2009/0062129 and US 2009/0191553. Exemplary next generating sequencing methods known to those of skill in the art include Massively parallel signature sequencing (MPSS), Polony sequencing, pyrosequencing (454), Illumina (Solexa) sequencing by synthesis, SOLiD sequencing by ligation, Ion semiconductor sequencing (Ion Torrent sequencing), DNA nanoball sequencing, chain termination sequencing (Sanger sequencing), Heliscope single molecule sequencing, Single molecule real time (SMRT) sequencing (Pacific Biosciences) and nanopore sequencing such as is described at world wide website nanoporetech.com.

Sequencing experiments can be done individually or in parallel to provide tens, hundreds, thousands, millions, or billions of sequences simultaneously. In addition to identifying standard nucleotides and amino acids, methods herein can identify natural and non-natural modifications within a sample, for example, methylation of the cytosine base to produce 5-methylcytosine. In some embodiments, a sample comprises modifications within traditional base pairs, such as hypoxanthine, or xanthine, which can be purine derivatives. In some embodiments, a sample can include natural and non-natural RNA derivatives, such as Inosine (I), and modifications such as 2′-O-methylribose modifications.

A computer program products and methods herein can analyze data generated by any sequencing instrument. Non-limiting examples of sequencers include: a) DNA sequencers produced by Illumina™, for example, HiSeg™, HiScanSQ™, Genome Analyzer GAIIX™ and MiSeg™ models; b) DNA sequencers produced by Life Technologies™, for example, DNA sequencers under the AB Applied Biosystems™ and/or Ion Torrent™ brands; c) DNA sequencers manufactured by Beckman Coulter™; d) DNA sequencers manufactured by 454 Life Sciences™; and e) DNA sequencers manufactured by Pacific Biosciences™.

Methods of the disclosure can analyze data obtained in a variety of sequencing experiments. For example, sequencing-by-synthesis technology uses a unique fluorescent-label for each of adenine, cytidine, guanine, and thymidine. A nucleotide chain is synthesized based on a sample sequence, and during each sequencing cycle, a single labeled deoxynucleoside triphosphate (dNTP) is added to the nucleic acid chain. The nucleotide label terminates polymerization. After each dNTP incorporation, the fluorescent dye is irradiated to identify the newly-added residue and then the label is enzymatically cleaved to allow incorporation of the next nucleotide.

Semiconductor sequencing technology is based on the premise that when a nucleotide is incorporated into a strand of DNA by a polymerase, a hydrogen ion is released as a byproduct. A sequence is synthesized based on a template sample sequence. Each of adenine, cytidine, guanine, and thymidine is added sequentially. If one of the nucleotides is incorporated into the growing chain, the charge from the released hydrogen ion changes the pH of the reaction solution. A solid-state pH meter detects the pH change, thereby identifying the chain elongation. If a nucleotide is not incorporated, no voltage change will be recorded by the solid-state pH meter and no information will be incorporated into a sequencing read. A pattern of released hydrogen ions can provide the information needed to construct a sequencing read.

Additional methods of sequencing are described in US Application No. 20150066385; Quail et al., 2012, BMC Genomics. 13 (1): 34; Liu et al., 2012, Journal of Biomedicine and Biotechnology. 2012: 1-11; each of which is incorporated herein by reference in its entirety.

While some exemplary methods for sequencing are provided herein, these are exemplary and not meant to limit the scope of the present disclosure. Additional suitable methods for sequencing will be apparent to those of skill in the art based on the present disclosure in view of the knowledge in the art.

All publications, patents and patent applications cited in this specification are herein incorporated by reference in their entirety for all purposes as if each individual publication, patent, or patent application were specifically and individually indicated to be incorporated by reference for all purposes. The publications discussed herein are provided solely for their disclosure prior to the filing date of the present application. Nothing herein is to be construed as an admission that the inventors described herein are not entitled to antedate such disclosure by virtue of prior disclosure or for any other reason.

The following Examples are set forth as being representative of the present disclosure. These examples are not to be construed as limiting the scope of the disclosure as these and other equivalent embodiments will be apparent in view of the present disclosure, figures, tables and accompanying claims.

### EXAMPLES

### Example 1: Exemplary Yeast Drug Screening Platform

In a classic high-throughput drug screen, a protein's enzymatic function is often linked to a reporter that typically produces a readily identifiable colorimetric change. Most often, a single protein is studied against an arrayed library of drugs. Antagonists are identified as compounds that inhibit the standard color change whereas agonists are identified as compounds that amplify the color change (Broach J R, et al., Nature 1996; 384:14-6; Szymanski P, et al., Int J Mol Sci. 2012; 13(1):427-52). Unfortunately, some proteins are not amenable to this type of screen as their enzymatic or cellular function is not easily tied to a colorimetric readout. However, the biggest limitation of this drug screening approach is that only a singular protein or protein complex is screened each time, limiting throughput to only one dimension of screening, i.e., the number of compounds screened.

As shown in the present Example, by linking a protein's natural enzymatic or cellular function to organism fitness, where expression of a protein of interest causes toxicity to the host cell, many different protein/protein families can be studied using a common screening output (e.g., growth). Furthermore, as described in the present Example, by inserting DNA barcodes into each of the cells and associating each of them with a particular protein, the effects of expressing a given protein within a population of cells can be tracked by simply reading out the abundance of its associated DNA barcode. Using this strategy, a pool of DNA barcoded cells can be generated and incubated with libraries of small compounds and, thus, the effects of the drug on dozens or hundreds of proteins of interest can be observed at a time in a single screen.

The present Example describes an exemplary screening platform utilizing Saccharomyces cerevisiae (yeast). Yeast are an ideal cellular chassis to perform the drug screening methods of the present disclosure, as they have tractable systems for genetic modification and are relatively inexpensive to maintain compared to other cellular models. However, the system as described in the present disclosure is not confined to yeast and is readily adaptable to work in any cellular system.

Methods

Yeast Strains and Media Conditions

All strains in the present study were derived from BY4741 (MATa his3Δ1 leu2Δ0 met15Δ0 ura3Δ0). To introduce the yeast expression vectors into the strain, a modified lithium acetate/single-stranded carrier DNA/PEG method of transformation was used (PMID:6336730). Briefly, strains to be transformed were grown overnight at 30° C. to saturation in YPD liquid suspension, and the saturated culture inoculated at 1:100 dilution in fresh YPD and grown for 4 hours on the day of transformation to achieve mid-log phase. Cells were centrifuged at 3220 g for 5 minutes, and washed in 1× LiOAc/TE (100 mM LiOAc, 10 mM Tris-HCl, 1 mM EDTA) before being added to the transformation mix (40% Peg3350, 1× LiOAc/TE, 85 ug/ml ssDNA, 10% DMSO) with 1 μg of the expression plasmid. The mixture was briefly vortexed and incubated at 42° C. for 20 minutes, washed with 1×PBS to remove the transformation mix, and plated on an agar plate of the appropriate Synthetic Complete (SC) selection medium.

Strains expressing the protein drug targets of interest were generated by transformation with the pAG416 or pAG426 series of vectors (Alberti S, et al., Yeast 2007; 24(10):913-9). The ORFs of the genes of interest were synthesized with flanking attB1 and attB2 sites in the 5′ and 3′ ends respectively, or amplified from a cDNA source using PCR primers including these sites (5′-GGGGACAAGTTTGTACAAAAAAGCAGGCTACAAAATG-3′ (SEQ ID NO:1) and 5′-GGGGACCACTTTGTACAAGAAAGCTGGGTTTTA-3′ (SEQ ID NO:2)). Table 1 provides the ORFs of selected proteases of interest. The ORF inserts were recombined into pDNOR221 entry vectors in a Gateway® BP reaction, and subsequently into the pAG vectors via LR reaction. The transformants were selected for and grown in the SC-ura medium.

TABLE 1
Open Reading Frames of Selected Proteases

| | SEQ ID | |
|---|---|---|
| Protease | NO: | ORF sequence |
| Rhinovirus 2A | 3 | ATGGGCCCCTCTGACATGTATGTGCATGTGGGAAACCTTATATACAGA |
| protease | | AACCTGCACTTGTTCAATAGTGAGATGCACGAGAGTATCCTCGTTTCCT |
| | | ACAGCAGTGACTTGATTATCTACAGGACTAATACCGTGGGCGATGACT |
| | | ACATACCGTCATGTGACTGCACACAGGCCACCTACTATTGTAAGCACA |
| | | AAAACCGCTATTTTCCCATCACTGTAACGTCCCATGATTGGTACGAGAT |
| | | TCAGGAAAGCGAGTATTATCCCAAACACATCCAATACAACTTGTTGAT |
| | | TGGAGAAGGCCCTTGTGAGCCTGGGGACTGTGGTGGCAAGCTCTTGTG |
| | | CAAACACGGAGTCATCGGTATAGTCACTGCGGGAGGCGATAACCATGT |
| | | CGCGTTCATTGACCTGCGACATTTCCACTGCGCCGAGGAACAGTAA |
| Poliovirus 2A | 4 | ATGGGGTTCGGGCACCAGAACAAGGCAGTTTATACTGCCGGCTACAAG |
| protease | | ATCTGTAACTATCACCTTGCAACGCAGGATGACTTGCAAAATGCTGTC |
| | | AATGTCATGTGGAGCCGCGACCTTTTGGTAACTGAGTCACGCGCCCAG |
| | | GGGACAGATAGTATAGCTAGGTGTAATTGCAACGCTGGAGTTTATTAT |
| | | TGCGAGTCACGCCGGAAATACTATCCAGTGAGTTTCGTGGGGCCTACT |
| | | TTTCAGTATATGGAGGCTAACAACTACTACCCAGCGCGGTATCAAAGC |
| | | CACATGCTTATCGGCCATGGGTTTGCCAGCCCTGGTGATTGTGGAGGC |
| | | ATACTCCGCTGCCATCACGGGGTAATCGGCATTATTACGGCTGGTGGT |
| | | GAAGGTCTGGTCGCGTTCTCTGACATTAGAGATCTTTACGCATATGAGG |
| | | AAGAAGCGATGGAACAGTAA |
| HIV-1 protease | 5 | ATGCCCCAAATAACACTCTGGCAAAGACCCCTTGTCACTATCAAGATT |
| | | GGAGGACAGCTCAAGGAGGCGTTGCTCGACACAGGGGCGGACGACAC |
| | | TGTTTTGGAGGAGATGTCTTTGCCGGGACGGTGGAAGCCGAAGATGAT |
| | | TGGAGGGATCGGTGGCTTTATTAAGGTGAGACAATACGATCAAATCCT |
| | | GATCGAGATATGCGGTCACAAGGCGATTGGGACGGTACTTGTAGGGCC |
| | | TACTCCCGTGAACATAATCGGGCGCAACCTTCTTACACAGATTGGTTGT |
| | | ACATTGAATTTTTAA |
| HIV-2 protease | 6 | ATGCCGCAATTCTCCCTGTGGAAAAGGCCTGTTGTGACCGCGTATATCG |
| | | AGGGGCAACCAGTTGAAGTTCTCCTGGACACTGGGGCTGATGACTCCA |
| | | TCGTCGCAGGTATTGAATTGGGAAATAACTACTCCCCTAAGATTGTGG |
| | | GGGGCATAGGAGGGTTCATCAACACAAAGGAGTACAAGAATGTTGAA |
| | | ATCGAGGTATTGAACAAAAAAGTCAGAGCAACCATTATGACAGGCGAT |
| | | ACGCCAATCAACATATTTGGTCGAAACATCTTGACGGCTTTGGGTATGT |
| | | CACTCAATCTCTAA |
| E. coli Lon | 7 | ATGAATCCTGAGCGTTCTGAACGCATTGAAATCCCCGTATTGCCGCTGC |
| protease | | GCGATGTGGTGGTTTATCCGCACATGGTCATCCCCTTATTTGTCGGGCG |
| | | GGAAAAATCTATCCGTTGTCTGGAAGCGGCGATGGACCATGATAAAAA |
| | | AATTATGCTGGTCGCGCAGAAAGAAGCTTCAACGGATGAGCCGGGTGT |
| | | AAACGATCTTTTCACCGTCGGGACCGTGGCCTCTATATTGCAGATGCTG |
| | | AAACTGCCTGACGGCACCGTCAAAGTGCTGGTCGAGGGGTTACAGCGC |
| | | GCGCGTATTTCTGCGCTCTCTGACAATGGCGAACACTTTTCTGCGAAGG |
| | | CGGAGTATCTGGAGTCGCCGACCATTGATGAGCGGGAACAGGAAGTGC |
| | | TGGTGCGTACTGCAATCAGCCAGTTCGAAGGCTACATCAAGCTGAACA |
| | | AAAAAATCCCACCAGAAGTGCTGACGTCGCTGAATAGCATCGACGATC |
| | | CGGCGCGTCTGGCGGATACCATTGCTGCACATATGCCGCTGAAACTGG |
| | | CTGACAAACAGTCTGTTCTGGAGATGTCCGACGTTAACGAACGTCTGG |
| | | AATATCTGATGGCAATGATGGAATCGGAAATCGATCTGCTGCAGGTTG |
| | | AGAAACGCATTCGCAACCGCGTTAAAAAGCAGATGGAGAAATCCCAG |
| | | CGTGAGTACTATCTGAACGAGCAAATGAAAGCTATTCAGAAAGAACTC |
| | | GGTGAAATGGACGACGCGCCGGACGAAAACGAAGCCCTGAAGCGCAA |
| | | AATCGACGCGGCGAAGATGCCGAAAGAGGCAAAAGAGAAAGCGGAA |
| | | GCAGAGTTGCAGAAGCTGAAAATGATGTCTCCGATGTCGGCAGAAGCG |
| | | ACCGTAGTGCGTGGTTATATCGACTGGATGGTACAGGTGCCGTGGAAT |
| | | GCGCGTAGCAAGGTCAAAAAAGACCTGCGTCAGGCGCAGGAAATCCTT |
| | | GATACCGACCATTATGGTCTGGAGCGCGTGAAAGATCGAATCCTTGAG |
| | | TATCTTGCGGTTCAAAGCCGTGTCAACAAAATCAAGGGACCGATCCTC |
| | | TGCCTGGTAGGGCCGCCGGGGGTAGGTAAAACCTCTCTTGGTCAGTCC |
| | | ATTGCCAAAGCCACCGGGCGTAAATATGTCCGTATGGCGCTGGGCGGC |
| | | GTGCGTGATGAAGCGGAAATCCGTGGTCACCGCCGTACTTACATCGGT |
| | | TCTATGCCGGGTAAACTGATCCAGAAAATGGCGAAAGTGGGCGTGAAA |
| | | AACCCGCTGTTCCTGCTCGATGAGATCGACAAAATGTCTTCTGACATGC |
| | | GTGGCGATCCGGCCTCTGCACTGCTTGAAGTGCTGGATCCAGAGCAGA |
| | | ACGTAGCGTTCAGCGACCACTACCTGGAAGTGGATTACGATCTCAGCG |
| | | ACGTGATGTTTGTCGCGACGTCGAACTCCATGAACATTCCGGCACCGCT |
| | | GCTGGATCGTATGGAAGTGATTCGCCTCTCCGGTTATACCGAAGATGA |
| | | AAAACTGAACATCGCCAAACGTCACCTGCTGCCGAAGCAGATTGAACG |
| | | TAATGCACTGAAAAAAGGTGAGCTGACCGTCGACGATAGCGCCATTAT |
| | | CGGCATTATTCGTTACTACACCCGTGAGGCGGGCGTGCGTGGTCTGGA |
| | | GCGTGAAATCTCCAAACTGTGTCGCAAAGCGGTTAAGCAGTTACTGCT |
| | | CGATAAGTCATTAAAACATATCGAAATTAACGGCGATAACCTGCATGA |
| | | CTATCTCGGTGTTCAGCGTTTCGACTATGGTCGCGCGGATAACGAAAA |
| | | CCGTGTCGGTCAGGTAACCGGTCTGGCGTGGACGGAAGTGGGCGGTGA |
| | | CTTGCTGACCATTGAAACCGCATGTGTTCCGGGTAAAGGCAAACTGAC |
| | | CTATACCGGTTCGCTCGGCGAAGTGATGCAGGAGTCCATTCAGGCGGC |
| | | GTTAACGGTGGTTCGTGCGCGTGCGGAAAAACTGGGGATCAACCCTGA |
| | | TTTTTACGAAAAACGTGACATCCACGTCCACGTACCGGAAGGTGCGAC |
| | | GCCGAAAGATGGTCCGAGTGCCGGTATTGCTATGTGCACCGCGCTGGT |
| | | TTCTTGCCTGACCGGTAACCCGGTTCGTGCCGATGTGGCAATGACCGGT |
| | | GAGATCACTCTGCGTGGTCAGGTACTGCCGATCGGTGGTTTGAAAGAA |
| | | AAACTCCTGGCAGCGCATCGCGGCGGGATTAAAACAGTGCTAATTCCG |
| | | TTCGAAAATAAACGCGATCTGGAAGAGATTCCTGACAACGTAATTGCC |
| | | GATCTGGACATTCATCCTGTGAAGCGCATTGAGGAAGTTCTGACTCTG |
| | | GCGCTGCAAAATGAACCGTCTGGTATGCAGGTTGTGACTGCAAAATAG |
| S. aureus V8 | 8 | ATGAAAGGTAAATTTTTAAAAGTTAGTTCTTTATTCGTTGCAACTTTGA |
| protease | | CAACAGCGACACTTGTGAGTTCTCCAGCAGCAAACGCGTTATCTTCAA |
| | | AGGCTATGGACAATCATCCACAACAAACGCAGTCAAGCAAACAGCAA |
| | | ACACCTAAGATTCAAAAAGGCGGTAACCTTAAACCATTAGAACAACGT |
| | | GAACACGCAAATGTTATATTACCAAATAACGATCGTCACCAAATCACA |
| | | GATACAACGAATGGTCATTATGCACCCGTAACTTATATTCAAGTTGAA |
| | | GCACCTACTGGTACATTTATTGCTTCCGGTGTAGTTGTAGGTAAAGATA |
| | | CTCTTTTAACAAATAAACACGTCGTAGATGCTACGCACGGTGATCCTCA |
| | | TGCTTTAAAAGCATTCCCTTCTGCAATTAACCAAGACAATTATCCAAAT |
| | | GGTGGTTTCACTGCTGAACAAATCACTAAATATTCAGGCGAAGGTGAT |
| | | TTAGCAATAGTTAAATTCTCCCCTAATGAGCAAAACAAACATATTGGT |
| | | GAAGTAGTTAAACCAGCAACAATGAGTAATAATGCTGAAACACAAGTT |
| | | AACCAAAATATTACTGTAACAGGATATCCTGGTGATAAACCTGTAGCA |
| | | ACAATGTGGGAAAGTAAAGGAAAAATCACTTACCTCAAAGGCGAAGC |
| | | TATGCAATATGATTTAAGTACAACTGGTGGTAATTCAGGTTCACCTGTA |
| | | TTTAATGAAAAAAATGAAGTGATCGGAATTCATTGGGGCGGTGTACCA |
| | | AATGAATTTAATGGTGCGGTATTTATTAATGAAAATGTACGCAACTTCT |
| | | TAAAACAAAATATTGAAGATATCCATTTTGCCAACGATGACCAACCTA |
| | | ATAACCCAGATAATCCTGATAACCCTAACAATCCTGATAACCCTAACA |
| | | ACCCAGATGAACCAAATAACCCTGACAACCCTAACAACCCTGATAATC |
| | | CAGACAATGGCGATAACAATAATTCAGACAATCCAGATGCAGCTTAA |
| S. aureus zinc | 9 | ATGGCAGCAACTGGCACAGGTAAAGGTGTGCTTGGAGATACAAAAGA |
| metallo- | | TATCAATATCAATAGTATTGATGGTGGATTTAGTTTAGAGGATTTGACG |
| proteinase | | CATCAAGGTAAATTATCAGCATACAATTTTAACGATCAAACAGGTCAA |
| | | GCGACATTAATTACTAATGAAGATGAAAACTTCGTCAAAGATGATCAA |
| | | CGTGCTGGTGTAGATGCGAATTATTATGCTAAACAAACATATGATTACT |
| | | ACAAAAATACATTTGGTCGTGAGTCTTACGATAACCATGGTAGTCCAA |
| | | TAGTCTCATTAACACATGTAAATCATTATGGTGGACAAGATAACAGAA |
| | | ATAACGCTGCATGGATTGGAGACAAAATGATTTATGGTGATGGCGATG |
| | | GCCGCACGTTTACAAATTTATCAGGTGCAAATGACGTAGTAGCACATG |
| | | AGTTAACACATGGCGTGACACAAGAAACGGCGAATTTAGAGTATAAA |
| | | GATCAATCTGGTGCGTTAAATGAAAGCTTTTCAGATGTTTTTGGATACT |
| | | TTGTAGATGATGAGGATTTCTTGATGGGTGAAGATGTTTACACACCAG |
| | | GAAAAGAGGGAGATGCTTTACGAAGCATGTCAAACCCAGAACAATTTG |
| | | GTCAACCATCTCATATGAAAGACTATGTATACACTGAAAAAGATAACG |
| | | GTGGTGTGCATACGAATTCTGGCATTCCAAATAAAGCAGCTTATAACG |
| | | TAATTCAAGCAATAGGGAAATCTAAATCAGAACAAATTTACTACCGAG |
| | | CATTAACGGAATACTTAACAAGTAATTCAAACTTCAAAGATTGTAAAG |
| | | ATGCATTATACCAAGCGGCTAAAGATTTATATGACGAGCAAACAGCTG |
| | | AACAAGTATATGAAGCATGGAACGAAGTTGGCGTCGAGTAA |
| V. cholerae | 10 | ATGAGCAATGCTTTTTTATAATGCCAACTTTGTACAAAAAAGCAGGCT |
| haemagglutinin | | ACAAAATGAAAATGATACAACGTCCTCTGAATTGGTTAGTTCTGGCCG |
| | | GAGCGGCAACTGGCTTCCCTCTCTATGCGGCACAAATGGTCACGATTG |
| | | ATGATGCATCAATGGTTGAACAAGCGTTGGCGCAGCAACAGTACAGTA |
| | | TGATGCCTGCCGCCAGCGGTTTTAAAGCCGTCAATACGGTACAGTTGC |
| | | CGAATGGTAAGGTGAAAGTGCGTTACCAGCAGATGTACAACGGGGTTC |
| | | CTGTCTATGGCACCGTTGTGGTGGCAACCGAGTCCAGTAAAGGGATTT |
| | | CGCAAGTGTATGGTCAAATGGCTCAGCAGTTGGAAGCCGATCTCCCAA |
| | | CCGTGACCCCTGACATTGAAAGCCAGCAGGCCATCGCTTTAGCGGTTA |
| | | GCCATTTTGGTGAACAACACGCTGGAGAATCGCTCCCGGTGGAAAACG |
| | | AAAGTGTGCAACTGATGGTACGTTTGGATGATAACCAACAGGCTCAGT |
| | | TAGTGTACTTGGTCGACTTTTTTGTCGCTTCAGAAACACCTTCGCGTCC |
| | | GTTCTACTTTATCAGTGCGGAAACGGGAGAAGTGCTAGACCAATGGGA |
| | | TGGCATTAACCACGCACAGGCAACAGGAACCGGCCCCGGCGGTAACC |
| | | AAAAAACGGGACGTTATGAATACGGCAGTAACGGTTTACCCGGTTTCA |
| | | CGATTGATAAGACCGGAACCACCTGTACGATGAATAACAGTGCGGTAA |
| | | AAACCGTTAACCTCAATGGCGGCACCTCGGGTAGCACGGCGTTCAGTT |
| | | ATGCTTGTAACAACAGCACTAACTACAACAGCGTGAAAACAGTGAATG |
| | | GTGCTTACTCACCGCTTAACGACGCGCACTTCTTTGGAAAAGTGGTGTT |
| | | TGATATGTATCAGCAGTGGTTGAATACTTCGCCGCTGACTTTCCAATTA |
| | | ACCATGCGTGTGCACTACGGCAATAACTATGAAAATGCCTTCTGGGAT |
| | | GGACGCGCCATGACTTTTGGTGATGGCTATACCCGTTTCTATCCTTTGG |
| | | TGGATATCAACGTTAGTGCCCATGAGGTCAGCCACGGTTTTACTGAGC |
| | | AGAATTCAGGCCTCGTTTACCGAGATATGTCCGGTGGTATTAACGAAG |
| | | CATTCTCGGATATCGCAGGGGAAGCGGCAGAGTACTTTATGCGTGGCA |
| | | ATGTCGACTGGATTGTCGGCGCGGATATTTTTAAATCCTCCGGTGGTCT |
| | | ACGTTATTTCGATCAGCCGTCACGTGATGGCCGCTCGATAGATCATGCT |
| | | TCACAGTATTACAGCGGTATTGATGTTCACCATTCGAGTGGCGTGTTTA |
| | | ACCGCGCGTTTTACCTACTCGCCAATAAATCGGGTTGGAACGTACGTA |
| | | AAGGTTTTGAAGTGTTTGCCGTGGCTAACCAGTTGTACTGGACACCGA |
| | | ACAGCACGTTTGATCAAGGTGGCTGTGGGGTAGTGAAAGCGGCGCAGG |
| | | ATCTCAACTACAACACCGCAGACGTTGTGGCAGCCTTTAATACCGTGG |
| | | GTGTCAATGCTTCTTGTGGCACCACGCCACCACCTGTCGGCAAAGTGCT |
| | | TGAGAAAGGTAAACCGATCACAGGACTGAGCGGCTCACGTGGAGGAG |
| | | AAGATTTCTATACCTTTACGGTGACCAATTCAGGCAGTGTTGTTGTGTC |
| | | CATCAGTGGTGGAACGGGCGATGCGGATCTGTATGTCAAAGCGGGCAG |
| | | CAAACCCACCACCTCTTCTTGGGATTGTCGTCCATACCGTTCAGGCAAT |
| | | GCCGAGCAGTGTTCCATCTCTGCGGTCGTGGGTACGACATACCATGTC |
| | | ATGTTACGCGGTTACAGTAACTATTCTGGTGTGACGTTACGCTTGGACT |
| | | AA |
| HIV VSV-G | 11 | ATGAAGTGCCTTTTGTACTTAGCCTTTTTATTCATTGGGGTGAATTGCA |
| | | AGTTCACCATAGTTTTTCCACACAACCAAAAAGGAAACTGGAAAAATG |
| | | TTCCTTCTAATTACCATTATTGCCCGTCAAGCTCAGATTTAAATTGGCA |
| | | TAATGACTTAATAGGCACAGCCTTACAAGTCAAAATGCCCAAGAGTCA |
| | | CAAGGCTATTCAAGCAGACGGTTGGATGTGTCATGCTTCCAAATGGGT |
| | | CACTACTTGTGATTTCCGCTGGTATGGACCGAAGTATATAACACATTCC |
| | | ATCCGATCCTTCACTCCATCTGTAGAACAATGCAAGGAAAGCATTGAA |
| | | CAAACGAAACAAGGAACTTGGCTGAATCCAGGCTTCCCTCCTCAAAGT |
| | | TGTGGATATGCAACTGTGACGGATGCCGAAGCAGTGATTGTCCAGGTG |
| | | ACTCCTCACCATGTGCTGGTTGATGAATACACAGGAGAATGGGTTGAT |
| | | TCACAGTTCATCAACGGAAAATGCAGCAATTACATATGCCCCACTGTC |
| | | CATAACTCTACAACCTGGCATTCTGACTATAAGGTCAAAGGGCTATGT |
| | | GATTCTAACCTCATTTCCATGGACATCACCTTCTTCTCAGAGGACGGAG |
| | | AGCTATCATCCCTGGGAAAGGAGGGCACAGGGTTCAGAAGTAACTACT |
| | | TTGCTTATGAAACTGGAGGCAAGGCCTGCAAAATGCAATACTGCAAGC |
| | | ATTGGGGAGTCAGACTCCCATCAGGTGTCTGGTTCGAGATGGCTGATA |
| | | AGGATCTCTTTGCTGCAGCCAGATTCCCTGAATGCCCAGAAGGGTCAA |
| | | GTATCTCTGCTCCATCTCAGACCTCAGTGGATGTAAGTCTAATTCAGGA |
| | | CGTTGAGAGGATCTTGGATTATTCCCTCTGCCAAGAAACCTGGAGCAA |
| | | AATCAGAGCGGGTCTTCCAATCTCTCCAGTGGATCTCAGCTATCTTGCT |
| | | CCTAAAAACCCAGGAACCGGTCCTGCTTTCACCATAATCAATGGTACC |
| | | CTAAAATACTTTGAGACCAGATACATCAGAGTCGATATTGCTGCTCCA |
| | | ATCCTCTCAAGAATGGTCGGAATGATCAGTGGAACTACCACAGAAAGG |
| | | GAACTGTGGGATGACTGGGCACCATATGAAGACGTGGAAATTGGACCC |
| | | AATGGAGTTCTGAGGACCAGTTCAGGATATAAGTTTCCTTTATACATGA |
| | | TTGGACATGGTATGTTGGACTCCGATCTTCATCTTAGCTCAAAGGCTCA |
| | | GGTGTTCGAACATCCTCACATTCAAGACGCTGCTTCGCAACTTCCTGAT |
| | | GATGAGAGTTTATTTTTTGGTGATACTGGGCTATCCAAAAATCCAATCG |
| | | AGCTTGTAGAAGGTTGGTTCAGTAGTTGGAAAAGCTCTATTGCCTCTTT |
| | | TTTCTTTATCATAGGGTTAATCATTGGACTATTCTTGGTTCTCCGAGTTG |
| | | GTATCCATCTTTGCATTAAATTAAAGCACACCAAGAAAAGACAGATTT |
| | | ATACAGACATAGAGATGAACCGACTTGGAAAGTAA |
| HIV gp160 | 12 | ATGAGGGACAATTGGAGAAGTGAATTATATAAATATAAAGTAGTAAA |
| | | AATTGAACCATTAGGAGTAGCACCCACCAAGGCAAAGAGAAGAGTGG |
| | | TGCAGAGAGAAAAAAGAGCAGTGGGAATAGGAGCTTTGTTCCTTGGGT |
| | | TCTTGGGAGCAGCAGGAAGCACTATGGGCGCAGCGTCAATGACGCTGA |
| | | CGGTACAGGCCAGACAATTATTGTCTGGTATAGTGCAGCAGCAGAACA |
| | | ATTTGCTGAGGGCTATTGAGGCGCAACAGCATCTGTTGCAACTCACAG |
| | | TCTGGGGCATCAAGCAGCTCCAGGCAAGAATCCTGGCTGTGGAAAGAT |
| | | ACCTAAAGGATCAACAGCTCCTGGGGATTTGGGGTTGCTCTGGAAAAC |
| | | TCATTTGCACCACTGCTGTGCCTTGGAATGCTAGTTGGAGTAATAAATC |
| | | TCTGGAACAGATTTGGAATCACACGACCTGGATGGAGTGGGACAGAGA |
| | | AATTAACAATTACACAAGCTTAATACACTCCTTAATTGAAGAATCGCA |
| | | AAACCAGCAAGAAAAGAATGAACAAGAATTATTGGAATTAGATAAAT |
| | | GGGCAAGTTTGTGGAATTGGTTTAACATAACAAATTGGCTGTGGTATA |
| | | TAAAATTATTCATAATGATAGTAGGAGGCTTGGTAGGTTTAAGAATAG |
| | | TTTTTGCTGTACTTTCTATAGTGAATAGAGTTAGGCAGGGATATTCACC |
| | | ATTATCGTTTCAGACCCACCTCCCAACCCCGAGGGGACCCGACAGGCC |
| | | CGAAGGAATAGAAGAAGAAGGTGGAGAGAGAGACAGAGACAGATCC |
| | | ATTCGATTAGTGAACGGATCCTTGGCACTTATCTGGGACGATCTGCGG |
| | | AGCCTGTGCCTCTTCAGCTACCACCGCTTGAGAGACTTACTCTTGATTG |
| | | TAACGAGGATTGTGGAACTTCTGGGACGCAGGGGGTGGGAAGCCCTCA |
| | | AATATTGGTGGAATCTCCTACAATATTGGAGTCAGGAGCTAAAGAATA |
| | | GTGCTGTTAGCTTGCTCAATGCCACAGCCATAGCAGTAGCTGAGGGGA |
| | | CAGATAGGGTTATAGAAGTAGTACAAGGAGCTTGTAGAGCTATTCGCC |
| | | ACATACCTAGAAGAATAAGACAGGGCTTGGAAAGGATTTTGCTATAA |
| HIV gag | 13 | ATGGGTGCGAGAGCGTCAGTATTAAGCGGGGGAGAATTAGATCGATG |
| | | GGAAAAAATTCGGTTAAGGCCAGGGGGAAAGAAAAAATATAAATTAA |
| | | AACATATAGTATGGGCAAGCAGGGAGCTAGAACGATTCGCAGTTAATC |
| | | CTGGCCTGTTAGAAACATCAGAAGGCTGTAGACAAATACTGGGACAGC |
| | | TACAACCATCCCTTCAGACAGGATCAGAAGAACTTAGATCATTATATA |
| | | ATACAGTAGCAACCCTCTATTGTGTGCATCAAAGGATAGAGATAAAAG |
| | | ACACCAAGGAAGCTTTAGACAAGATAGAGGAAGAGCAAAACAAAAGT |
| | | AAGAAAAAAGCACAGCAAGCAGCAGCTGACACAGGACACAGCAATCA |
| | | GGTCAGCCAAAATTACCCTATAGTGCAGAACATCCAGGGGCAAATGGT |
| | | ACATCAGGCCATATCACCTAGAACTTTAAATGCATGGGTAAAAGTAGT |
| | | AGAAGAGAAGGCTTTCAGCCCAGAAGTGATACCCATGTTTTCAGCATT |
| | | ATCAGAAGGAGCCACCCCACAAGATTTAAACACCATGCTAAACACAGT |
| | | GGGGGGACATCAAGCAGCCATGCAAATGTTAAAAGAGACCATCAATG |
| | | AGGAAGCTGCAGAATGGGATAGAGTGCATCCAGTGCATGCAGGGCCT |
| | | ATTGCACCAGGCCAGATGAGAGAACCAAGGGGAAGTGACATAGCAGG |
| | | AACTACTAGTACCCTTCAGGAACAAATAGGATGGATGACACATAATCC |
| | | ACCTATCCCAGTAGGAGAAATCTATAAAAGATGGATAATCCTGGGATT |
| | | AAATAAAATAGTAAGAATGTATAGCCCTACCAGCATTCTGGACATAAG |
| | | ACAAGGACCAAAGGAACCCTTTAGAGACTATGTAGACCGATTCTATAA |
| | | AACTCTAAGAGCCGAGCAAGCTTCACAAGAGGTAAAAAATTGGATGA |
| | | CAGAAACCTTGTTGGTCCAAAATGCGAACCCAGATTGTAAGACTATTT |
| | | TAAAAGCATTGGGACCAGGAGCGACACTAGAAGAAATGATGACAGCA |
| | | TGTCAGGGAGTGGGGGGACCCGGCCATAAAGCAAGAGTTTTGGCTGAA |
| | | GCAATGAGCCAAGTAACAAATCCAGCTACCATAATGATACAGAAAGG |
| | | CAATTTTAGGAACCAAAGAAAGACTGTTAAGTGTTTCAATTGTGGCAA |
| | | AGAAGGGCACATAGCCAAAAATTGCAGGGCCCCTAGGAAAAAGGGCT |
| | | GTTGGAAATGTGGAAAGGAAGGACACCAAATGAAAGATTGTACTGAG |
| | | AGACAGGCTAATTTTTTAGGGAAGATCTGGCCTTCCCACAAGGGAAGG |
| | | CCAGGGAATTTTCTTCAGAGCAGACCAGAGCCAACAGCCCCACCAGAA |
| | | GAGAGCTTCAGGTTTGGGGAAGAGACAACAACTCCCTCTCAGAAGCAG |
| | | GAGCCGATAGACAAGGAACTGTATCCTTTAGCTTCCCTCAGATCACTCT |
| | | TTGGCAGCGACCCCTCGTCACAATAA |
| HIV Integrase | 14 | ATGTTTTTAGATGGAATAGATAAGGCCCAAGAAGAACATGAGAAATAT |
| | | CACAGTAATTGGAGAGCAATGGCTAGTGATTTTAACCTACCACCTGTA |
| | | GTAGCAAAAGAAATAGTAGCCAGCTGTGATAAATGTCAGCTAAAAGG |
| | | GGAAGCCATGCATGGACAAGTAGACTGTAGCCCAGGAATATGGCAGCT |
| | | AGATTGTACACATTTAGAAGGAAAAGTTATCTTGGTAGCAGTTCATGT |
| | | AGCCAGTGGATATATAGAAGCAGAAGTAATTCCAGCAGAGACAGGGC |
| | | AAGAAACAGCATACTTCCTCTTAAAATTAGCAGGAAGATGGCCAGTAA |
| | | AAACAGTACATACAGACAATGGCAGCAATTTCACCAGTACTACAGTTA |
| | | AGGCCGCCTGTTGGTGGGCGGGGATCAAGCAGGAATTTGGCATTCCCT |
| | | ACAATCCCCAAAGTCAAGGAGTAATAGAATCTATGAATAAAGAATTAA |
| | | AGAAAATTATAGGACAGGTAAGAGATCAGGCTGAACATCTTAAGACA |
| | | GCAGTACAAATGGCAGTATTCATCCACAATTTTAAAAGAAAAGGGGGG |
| | | ATTGGGGGGTACAGTGCAGGGGAAAGAATAGTAGACATAATAGCAAC |
| | | AGACATACAAACTAAAGAATTACAAAAACAAATTACAAAAATTCAAA |
| | | ATTTTCGGGTTTATTACAGGGACAGCAGAGATCCAGTTTGGAAAGGAC |
| | | CAGCAAAGCTCCTCTGGAAAGGTGAAGGGGCAGTAGTAATACAAGAT |
| | | AATAGTGACATAAAAGTAGTGCCAAGAAGAAAAGCAAAGATCATCAG |
| | | GGATTATGGAAAACAGATGGCAGGTGATGATTGTGTGGCAAGTAGACA |
| | | GGATGAGGATTAATAA |
| E. coli ClpX | 15 | ATGAGCAATGCTTTTTTATAATGCCAACTTTGTACAAAAAAGCAGGCT |
| | | ACAAAATGACAGATAAACGCAAAGATGGCTCAGGCAAATTGCTGTATT |
| | | GCTCTTTTTGCGGCAAAAGCCAGCATGAAGTGCGCAAGCTGATTGCCG |
| | | GTCCATCCGTGTATATCTGCGACGAATGTGTTGATTTATGTAACGACAT |
| | | CATTCGCGAAGAGATTAAAGAAGTTGCACCGCATCGTGAACGCAGTGC |
| | | GCTACCGACGCCGCATGAAATTCGCAACCACCTGGACGATTACGTTAT |
| | | CGGCCAGGAACAGGCGAAAAAAGTGCTGGCGGTCGCGGTATACAACC |
| | | ATTACAAACGTCTGCGCAACGGCGATACCAGCAATGGCGTCGAGTTGG |
| | | GCAAAAGTAACATTCTGCTGATCGGTCCGACCGGTTCCGGTAAAACGC |
| | | TGCTGGCTGAAACGCTGGCGCGCCTGCTGGATGTTCCGTTCACCATGGC |
| | | CGACGCGACTACACTGACCGAAGCCGGTTATGTGGGTGAAGACGTTGA |
| | | AAACATCATTCAGAAGCTGTTGCAGAAATGCGACTACGATGTCCAGAA |
| | | AGCACAGCGTGGTATTGTCTACATCGATGAAATCGACAAGATTTCTCG |
| | | TAAGTCAGACAACCCGTCCATTACCCGAGACGTTTCCGGTGAAGGCGT |
| | | ACAGCAGGCACTGTTGAAACTGATCGAAGGTACGGTAGCTGCTGTTCC |
| | | ACCGCAAGGTGGGCGTAAACATCCGCAGCAGGAATTCTTGCAGGTTGA |
| | | TACCTCTAAGATCCTGTTTATTTGTGGCGGTGCGTTTGCCGGTCTGGAT |
| | | AAAGTGATTTCCCACCGTGTAGAAACCGGCTCCGGCATTGGTTTTGGC |
| | | GCGACGGTAAAAGCGAAGTCCGACAAAGCAAGCGAAGGCGAGCTGCT |
| | | GGCGCAGGTTGAACCGGAAGATCTGATCAAGTTTGGTCTTATCCCTGA |
| | | GTTTATTGGTCGTCTGCCGGTTGTCGCAACGTTGAATGAACTGAGCGAA |
| | | GAAGCTCTGATTCAGATCCTCAAAGAGCCGAAAAACGCCCTGACCAAG |
| | | CAGTATCAGGCGCTGTTTAATCTGGAAGGCGTGGATCTGGAATTCCGT |
| | | GACGAGGCGCTGGATGCTATCGCTAAGAAAGCGATGGCGCGTAAAAC |
| | | CGGTGCCCGTGGCCTGCGTTCCATCGTAGAAGCCGCACTGCTCGATAC |
| | | CATGTACGATCTGCCGTCCATGGAAGACGTCGAAAAAGTGGTTATCGA |
| | | CGAGTCGGTAATTGATGGTCAAAGCAAACCGTTGCTGATTTATGGCAA |
| | | GCCGGAAGCGCAACAGGCATCTGGTGAATAA |
| Human LINE- | 16 | ATGGGAATGCTGGAGCTGCGGATCAAGAACCTGACCCAGAGCCGGAG |
| 1 retrotrans- | | CACCACCTGGAAGCTGAACAACCTGCTGCTGAACGACTACTGGGTtCAC |
| poson ORF2 | | AACGAGATGAAGGCCGAGATCAAGATGTTCTTCGAGACCAACGAGAA |
| | | CAAGGACACCACCTACCAGAACCTGTGGGACGCCTTCAAGGCCGTGTG |
| | | CCGGGGCAAGTTCATCGCCCTGAACGCCTACAAGCGGAAGCAGGAGC |
| | | GGAGCAAGATCGACACCCTGACCAGCCAGCTGAAGGAGCTGGAGAAG |
| | | CAGGAGCAGACCCACAGCAAGGCCAGtCGGCGGCAGGAGATCACCAA |
| | | GATCCGGGCCGAGCTGAAGGAGATCGAGACCCAGAAGACCCTtCAGAA |
| | | GATCAACGAGAGCCGGAGCTGGTTCTTCGAGCGGATCAACAAGATCGA |
| | | CCGGCCCCTGGCCCGGCTGATCAAGAAGAAGCGGGAGAAGAACCAGA |
| | | TCGACACCATCAAGAACGACAAGGGCGACATCACCACCGACCCCACCG |
| | | AGATCCAGACCACCATCCGGGAGTACTACAAGCACCTGTACGCCAACA |
| | | AGCTGGAGAAtCTaGAGGAGATGGACACCTTCCTGGACACCTACACCCT |
| | | GCCCCGGCTGAACCAGGAGGAGGTGGAGAGCCTGAACCGGCCCATCA |
| | | CCGGCAGCGAGATCGTGGCCATCATCAACAGCCTGCCCACCAAGAAGA |
| | | GCCCCGGCCCCGACGGCTTCACCGCCGAGTTCTACCAGCGGTACAAGG |
| | | AGGAGCTGGTGCCCTTCCTGCTGAAGCTGTTCCAGAGCATCGAGAAGG |
| | | AGGGCATCCTGCCCAACAGCTTCTACGAGGCCAGCATCATCCTGATCC |
| | | CCAAGCCCGGCCGGGACACCACCAAGAAGGAGAACTTCCGGCCCATC |
| | | AGCCTGATGAACATCGACGCCAAGATCCTGAACAAGATCCTGGCCAAC |
| | | CGGATtCAGCAGCACATCAAGAAGCTGATCCACCACGACCAaGTtGGCT |
| | | TCATCCCCGGCATGCAaGGaTGGTTCAACATCCGGAAGAGCATCAACGT |
| | | GATCCAGCACATCAACCGGGCCAAGGACAAGAACCACATGATCATCA |
| | | GCATCGACGCCGAGAAGGCtTTCGACAAGATCCAGCAGCCCTTCATGCT |
| | | GAAGACCCTGAACAAGCTGGGCATCGACGGCACCTACTTCAAGATCAT |
| | | CCGGGCCATCTACGACAAGCCCACCGCCAACATCATCCTGAACGGCCA |
| | | GAAGCTGGAGGCtTTCCCCCTGAAGACCGGCACCCGGCAGGGCTGCCC |
| | | CCTGAGCCCCCTGCTGTTCAACATCGTGCTGGAGGTGCTGGCCCGGGC |
| | | CATCCGGCAGGAGAAGGAGATCAAGGGCATCCAGCTGGGCAAGGAGG |
| | | AGGTGAAGCTGAGCCTGTTCGCCGACGACATGATCGTGTACCTGGAGA |
| | | ACCCtATtGTtAGCGCCCAGAACCTGCTGAAGCTGATCAGCAACTTCAGt |
| | | AAaGTtAGCGGCTACAAGATCAACGTGCAGAAGAGCCAGGCtTTCCTGT |
| | | ACACCAAtAACCGGCAGACCGAGAGCCAGATCATGGGCGAGCTGCCCT |
| | | TCACCATCGCCAGCAAGCGGATCAAGTACCTGGGCATCCAGCTGACtCG |
| | | GGACGTcAAGGACCTGTTCAAGGAGAACTACAAGCCCCTGCTGAAGGA |
| | | GATCAAGGAGGAGACCAACAAGTGGAAGAACATCCCCTGtAGCTGGGT |
| | | GGGCCGGATCAACATCGTGAAGATGGCCATCCTGCCgAAaGTtATCTAtC |
| | | GGTTCAACGCCATCCCCATCAAGCTGCCCATGACCTTCTTCACCGAGCT |
| | | GGAGAAGACCACCCTGAAGTTCATCTGGAACCAGAAGCGGGCCCGGA |
| | | TCGCCAAGAGCATCCTGAGCCAGAAGAACAAGGCCGGCGGCATCACC |
| | | CTGCCCGACTTCAAGCTGTACTACAAGGCCACCGTGACCAAGACCGCC |
| | | TGGTACTGGTACCAGAACCGGGACATCGACCAGTGGAACCGGACCGA |
| | | GCCCAGCGAGATCATGCCCCACATCTACAACTACCTGATCTTCGACAA |
| | | GCCCGAGAAGAACAAGCAGTGGGGCAAGGACAGCCTGTTCAACAAGT |
| | | GGTGCTGGGAGAACTGGCTGGCCATCTGCCGGAAGCTGAAGCTGGACC |
| | | CCTTCCTGACCCCCTACACCAAGATCAACAGCCGGTGGATCAAGGACC |
| | | TGAACGTGAAGCCCAAGACCATCAAGACCCTGGAGGAGAACCTGGGC |
| | | ATCACCATCCAGGACATCGGCGTGGGCAAGGACTTCATGAGCAAGACC |
| | | CCCAAGGCcATGGCCACCAAGGACAAGATCGACAAGTGGGACCTGATC |
| | | AAGCTGAAGAGCTTCTGCACCGCCAAGGAGACCACCATCCGGGTGAAC |
| | | CGGCAGCCCACCACCTGGGAGAAGATtTTCGCCACCTACAGCAGCGAC |
| | | AAGGGCCTGATCAGCCGGATCTACAACGAGCTGAAGCAGATtTACAAG |
| | | AAGAAGACCAACAACCCCATCAAGAAGTGGGCCAAGGACATGAACCG |
| | | GCACTTCAGCAAGGAGGACATCTACGCCGCCAAGAAGCACATGAAGA |
| | | AGTGCAGCAGCAGCCTGGCCATCCGGGAGATGCAGATCAAGACCACC |
| | | ATGCGGTAtCACCTGACCCCCGTGCGGATGGCCATCATCAAGAAGAGC |
| | | GGCAACAACCGaTGCTGGCGGGGCTGCGGCGAGATCGGCACCCTGCTG |
| | | CACTGCTGGTGGGACTGCAAGCTGGTGCAGCCCCTGTGGAAGAGCGTG |
| | | TGGCGGTTCCTGCGGGACCTGGAGCTGGAGATCCCCTTCGACCCCGCC |
| | | ATCCCCCTGCTGGGCATCTACCCCAACGAGTACAAGAGCTGCTGCTAC |
| | | AAGGACACCTGCACCCGGATGTTCATCGCCGCCCTGTTCACCATCGCC |
| | | AAGACCTGGAACCAGCCCAAGTGCCCCACCATGATCGACTGGATCAAG |
| | | AAGATGTGGCACATCTACACtATGGAGTACTACGCCGCCATCAAGAAC |
| | | GACGAGTTCATCAGCTTCGTGGGCACCTGGATGAAGCTGGAGACCATC |
| | | ATCCTGAGCAAGCTGAGCCAGGAGCAGAAGACCAAGCACCGGATCTTC |
| | | AGCCTGATCGGCGGCAACTAA |

Development of Barcode Plasmids

To generate DNA barcoded libraries, the pAG415-GAL-ccdB vector was used as the backbone to insert a kanamycin resistance gene (Kan) ORF with a 10 bp random barcode downstream of the sequence. The Kan promoter and ORF were amplified from a cDNA source using primers that add a N(10) barcode sequence to the PCR product along with SacI and NgoMIV restriction cut sites. The PCR product was then digested with SacI and NgoMIV and gel purified. The pAG415-GAL-ccdB vector was digested with SacI, NgoMIV, and NcoI enzymes and the 5 kB backbone product was gel purified. The two purified products were ligated using T4 DNA ligase. The ligation product was column purified and electroporated into NEB 10-beta electrocompetent cells, and plated on Amp agar plates. The grown colonies were harvested using a cell scraper to create the final library pool which was frozen down. Single colonies were then obtained from the cell slurry and barcoded plasmids from these cells were sequenced and used in the subsequent experiments.

Knockout of Drug Transporters

Knockout strains of yeast multidrug resistance regulating transcription factors, PDR1 and PDR3, and the multidrug exporter SNQ2 were derived using CRISPR-mediated methods (Nageshwaran S, et al., J Vis Exp. 2018; 140; Bao Z, et al., ACS Synth Biol. 2015; 4(5):585-94). gRNA sequences were selected using the Broad sgRNA design tool (http.//portals.broadinstitute.org/gpp/public/analysis-tools/sgma-design) against the ORF of the above genes, the sequences GATC and AAAC were attached to the 5′ end of the gRNA sequence and its reverse complement, respectively, and cloned into the pCRCT vectors modified to be golden gate cloning compatible. A donor oligo was also designed to extend 45 bases upstream of the start codon of the ORF, and 45 bases downstream of the stop codon, to be a total of 90 bps in length. The above LiOAc transformation protocol was used to introduce 1 μg of the pCRCT plasmid for specific genes along with 10 μL of a 100 μM solution of the corresponding donor oligo. Transformants were selected on SC-ura agar plates, and the grown colonies were confirmed for knockout via colony PCR using primers upstream and downstream of the deleted ORF. Colonies confirmed for knockout were then grown on 5-FOA plates to select for the loss of the pCRCT plasmid, and subsequently grown in YPD.

Extraction of DNA from Yeast

A standard LiOAC-SDS based protocol was used to extract DNA from yeast cultures for library preparation (Lõoke M, et al., Biotechniques 2011; 50(5):325-8). Briefly, saturated yeast cultures were pelleted and resuspended in 200 μL of 200 mM LiOAc solution with 1% SDS and were incubated at 65 degrees celsius for 20 minutes. Next, 3× volume of 100% Ethanol was added before centrifugation for 10 minutes at 4000 rpm. The supernatant was decanted and the pellet containing DNA was allowed to air dry for 15 minutes. The pellet was resuspended in 1×TE (10 mM Tris-HCl, 1 mM EDTA) and then incubated at 42 degrees celsius for 30 minutes. After centrifugation for 10 minutes at 4000 rpm, the supernatant containing genomic and plasmid DNA was saved for subsequent PCR and library preparation.

Drug Screen Conditions and Set Up

All drugs were screened at a concentration of 10 μM. Barcoded cells expressing various protein drug targets were grown up individually on agar plates containing SC-ura-leu glucose. These strains were then inoculated into SC-ura-leu with glucose liquid culture media and after overnight growth were mixed together in equal amounts to generate the DNA barcoded library. The DNA barcoded library was then inoculated into SC-ura-leu with galactose liquid culture media with and without small molecule compounds.

Library Preparation for Next-Generation Sequencing (NGS)

Amplicons for NGS were generated through a two-step PCR protocol. Barcode sequences were amplified with Taq polymerase from genomic DNA preparations with primers anchored in constant regions of the 415-Kan-BC plasmid. During this amplification step, forward primers contained an internal barcode upstream of the binding sequence for demultiplexing. DNA barcodes were amplified for 28 cycles in the first round. After first round amplification, amplicons were diluted 1/40 and amplified with primers containing Illumina compatible sequences for 8 cycles. Finally, all samples were pooled into a single library, which was gel purified. Amplicons were then subjected to standard single-ended sequencing on an Illumina Sequencer.

Analysis Pipeline

Raw sequencing reads were demultiplexed and attributed to each tested condition by the unique combination of an internal barcode and two Illumina indexes, and then converted to fasta format. Sequencing reads were trimmed to only include unique barcode sequence. Barcode counts were generated for each condition by aligning reads to a barcode reference with bowtie2. Count tables were filtered to remove barcodes that were lowly represented (<4 reads per condition) before further analysis. Barcode enrichment and depletion was determined for each condition with the MAGeCK algorithm. Top hits were identified by sorting model and condition interactions by positive False Discovery Rate (posfdr).

Results and Discussion

It has been demonstrated for multiple native yeast proteins that overexpression results in cellular toxicity (Moriya H, Mol Biol Cell. 2015; 26(22):3932-9; Eguchi Y, et al., Elife. 2018 Aug. 10; 7. pii: e34595). However, this idea has been expanded by the inventors by demonstrating that a wide range of non-native and clinically-relevant protein classes, when overexpressed, also caused toxicity. The observed toxicity may be due to, for example, the biological functions inherent to the overexpressed proteins, and this toxicity could serve as the basis of a novel multiplex drug screening system.

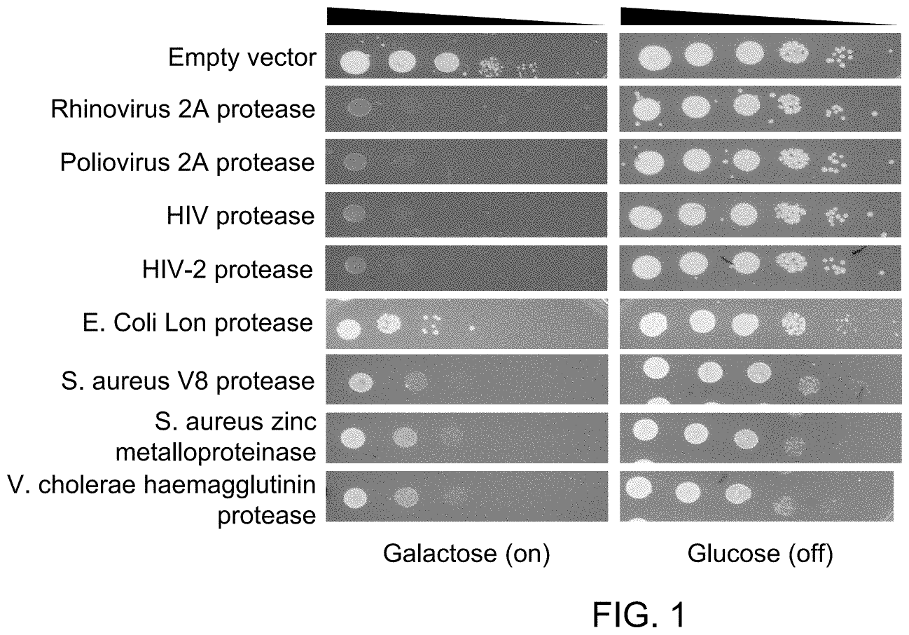

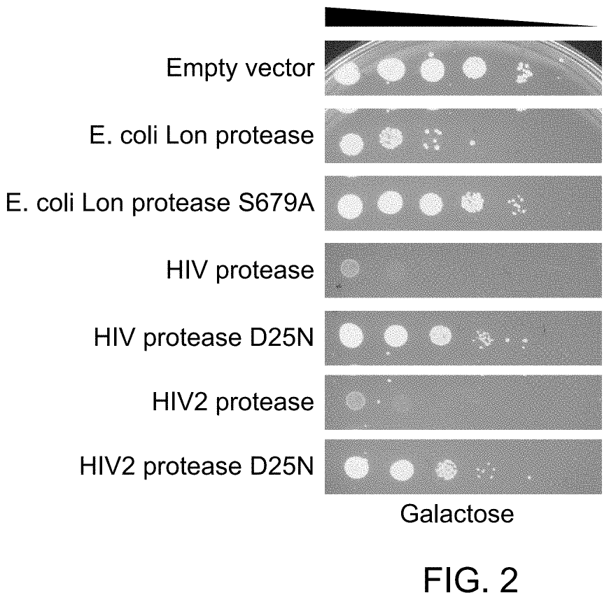

Initially, expression of proteases from a wide range of bacterial and viral origin was shown to result in toxicity to yeast cells (see FIG. 1). Subsequently, this toxicity was shown to be a result of their natural enzymatic activity through the mutation of active site residues that disable their enzymatic function and observing a lack of toxicity (see FIG. 2).

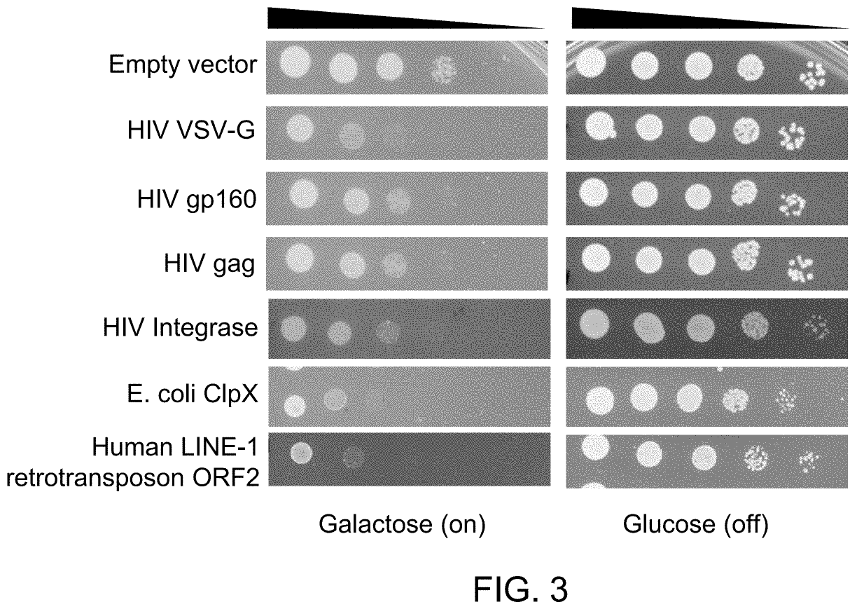

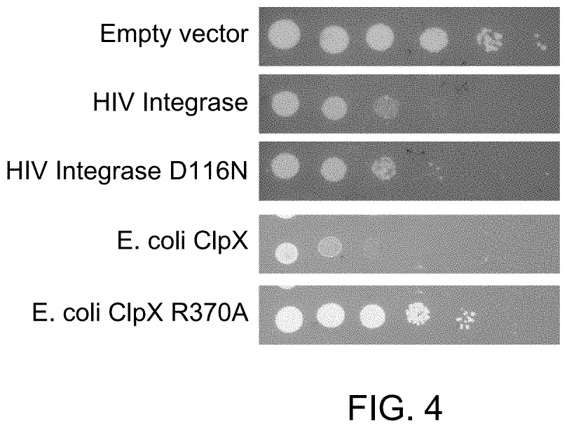

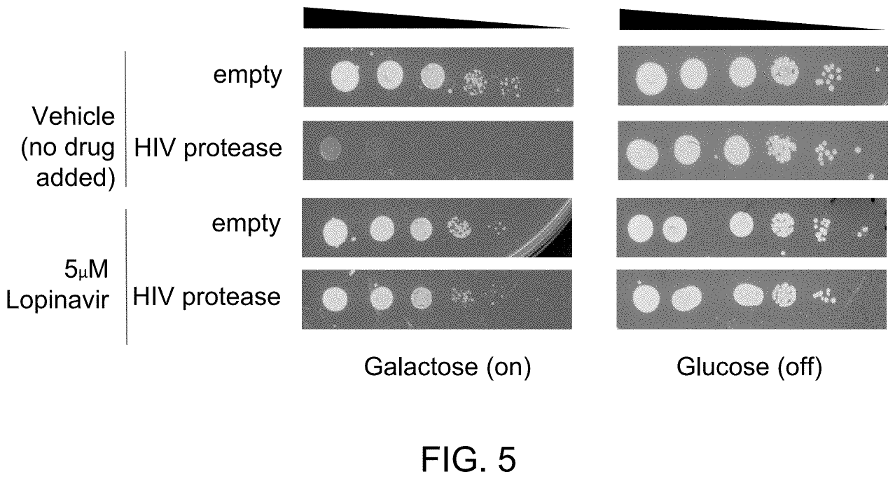

Furthermore, as demonstrated in FIG. 3, the screening methods of the present disclosure are not limited to a specific class of proteins such as proteases. Other classes of proteins, such as viral integrases, structural proteins, protein chaperones and reverse transcriptases, also caused toxicity to yeast. In addition, when tested, their growth suppression was dependent upon their natural biological function (see FIG. 4). Next, the toxicity of the yeast cell was demonstrated to be a suitable paradigm to screen for drugs that inhibit or alter the cellular function of the protein being expressed by showing that the toxicity can be mitigated by growing yeast in the presence of a small molecule inhibitor (Lopinavir™) specific to the protease (HIV protease) being expressed (see FIG. 5).

Having established the association between protein toxicity and biological function, along with demonstrating that small molecules which inhibit a given protein's biological activity could rescue the growth defects caused by its overexpression, the scale at which the small molecule screens could be performed was increased. In order to drastically increase the efficiency of drug screening, a strategy was devised for a mixed pool drug screening assay. In this approach, each yeast strain tested contained a unique DNA barcode. These yeast were then transformed with a given protein of interest which small molecule inhibitors were to be identified against. Then, hundreds of barcoded cell lines were mixed together and examined for their growth rate in the presence or absence of a given small molecule.

This strategy allows for the individual fitness of hundreds of yeast, each expressing a unique toxic protein, to be tracked, with internal replicates for statistical reliability made possible by associating each individual toxic protein with several unique DNA barcodes. After individually barcoding yeast strains expressing a toxic protein, they can be pooled and the relative abundance of each toxic protein expressing strain can be determined via next generation sequencing.

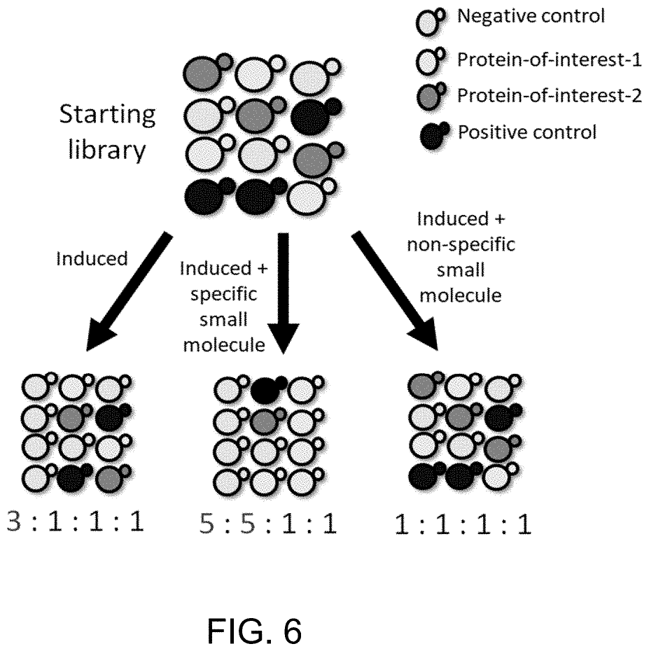

In order to maintain the relative abundance of barcoded strains at a consistent level before screening, toxic proteins were expressed under the control of an inducible promoter. Upon growth in inducing conditions, barcodes associated with the expression of toxic proteins became depleted in a mixed pool due to their reduced growth rate (see FIG. 6, induced). Introducing a drug to the inducing conditions allowed for the identification of barcodes that became enriched as compared to induction with no drug, signaling a protein-drug interaction (see FIG. 6, induced+specific small molecule). Additionally, control strains can also be introduced into the barcode pool to detect drug specificity. Negative control proteins that have no toxic effect on the cell, such as EYFP, can serve as a reference for barcode depletion in inducing conditions. Positive control proteins that have toxic effects on cells, but through mechanisms believed to be orthogonal to the class of proteins being screened, can serve to identify drugs that have non-specific effects, such as modulating or interfering with the genetic systems used to induce toxic protein expression (see FIG. 6, induced+non-specific small molecule).

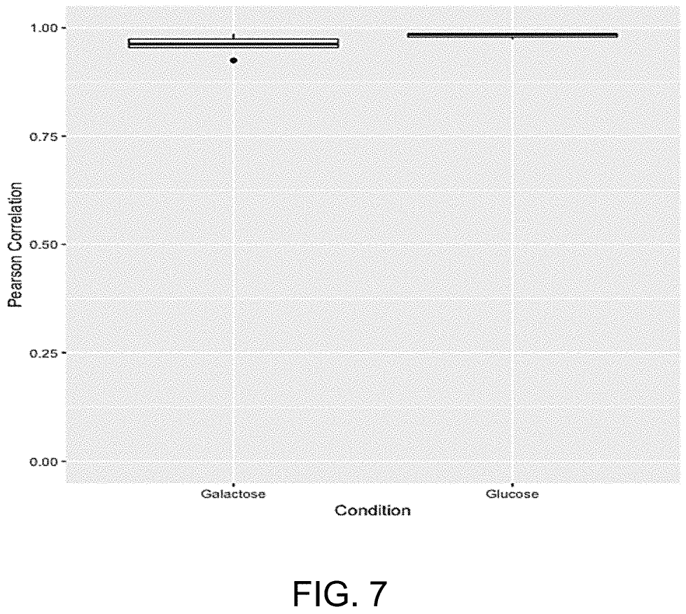

As demonstrated in FIG. 7, for pools containing dozens of barcodes, cells maintained their relative abundance before induction and skew according to their growth rate upon toxic protein expression in a highly reproducible fashion, allowing for a robust, negative-binomial based statistical test to determine enrichment in drug conditions (Li W, et al., Genome Biol. 2014; 15(12):554).

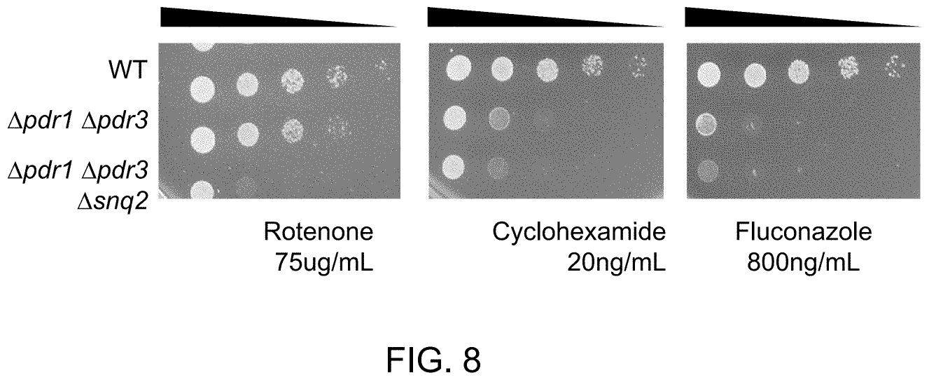

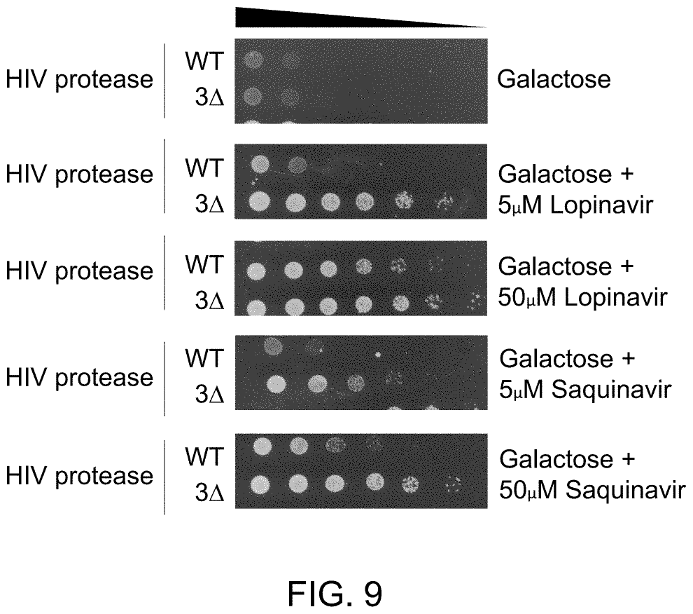

Standard drug screening conditions typically test protein-drug interactions at a single drug concentration (range 1-10 μM), potentially missing the discovery of protein-drug interactions that occur at a drug concentration outside of the tested concentration (Hughes J P, et al., Br J Pharmacol. 2011; 162(6):1239-49). Prior studies of yeast drug exporters have determined that lowering the expression of a limited, well-characterized, set of pleiotropic multidrug exporters in yeast results in higher concentrations of a drug in the cell at steady state (Rogers B, et al., J Mol Microbiol Biotechnol. 2001; 3(2):207-14; Kolaczkowski M, et al., Microb Drug Resist. 1998; 4(3):143-58; Piotrowski J S, et al., Nat Chem Biol. 2017; 13(9):982-993). Subsequently, knocking out these drug transporters resulted in variable sensitivity of the yeast to exogenous compounds (see FIG. 8). By expressing a toxic protein within a series of yeast strains, ranging from no constitutive drug exporter expression to high constitutive drug exporter expression, the concentration of drug introduced into the cell was modulated where it could interact with its toxic protein target without the need to change the amount of drug introduced into the well in which the screening took place. For example, expressing HIV protease within two strains, one with limited ability to pump a protease inhibitor into the extracellular environment (e.g., Δpdr1 Δpdr3 Δsnq2 cells), and another with normal ability to pump the protease inhibitor out of the cell (e.g., wild type cells) allowed for the determination of dose-response information from the degree of rescue observed (see FIG. 9).

DNA barcodes provide a high degree of scalability and can be assigned to track the fitness of a specific toxic-protein expressing cell in a range of cellular backgrounds, each with a different ability to export exogenous compounds. Thus, by looking at barcode abundance for all cells expressing a given toxic-protein and knowing which cells are designed to accumulate more or less of the introduced compound, dose-response information can be obtained despite adding the drug at a single concentration to the mixed pool of cells. Furthermore, more than two strains can be exploited to further improve the dose-response information obtained.

Finally, as an additional feature of the developed drug-screening methods as described in the present disclosure, other barcoded strains can be introduced into the mixed pool that can detect undesirable off-target drug effects or toxicities. To detect off-target toxicities, a series of gene knockouts were identified which were highly sensitized to minor disruptions in a range of cellular pathways such as DNA-damage or mitotic blockade (Piotrowski J S, et al., Nat Chem Biol. 2017; 13(9):982-993). By tracking the behavior of these mutants within the pool of cells when treated with each of the tested compounds, valuable information regarding the potential off-target activities that a tested compound can be obtained.

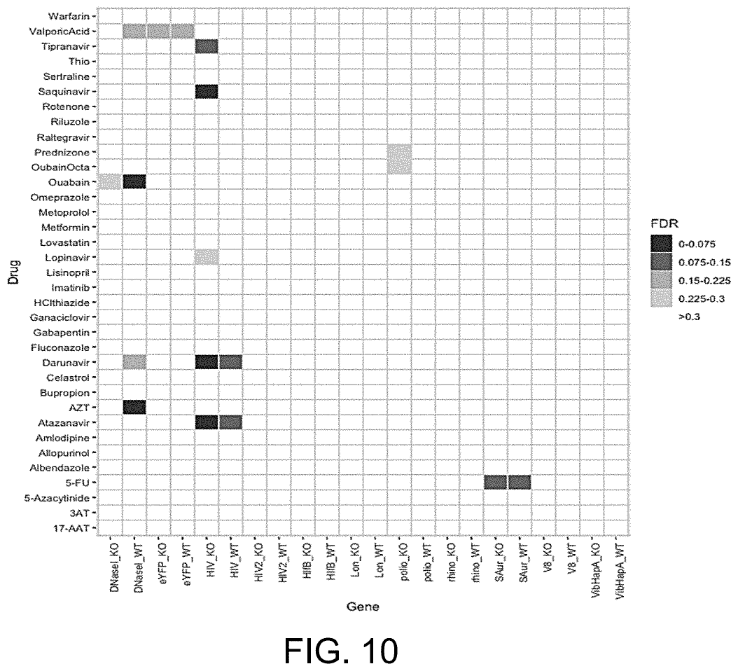

To demonstrate the application of the presently disclosed screening methods, a drug screen of 44 barcoded cell lines was conducted against 37 drugs. The barcoded cell lines consisted of 11 proteins, 9 of which were proteases of viral or bacterial origin, and two control proteins. The negative control protein, EYFP, is a non-toxic fluorescent protein. The positive control protein within this screen is DNAseI, a protein that non-specifically cleaves DNA and causes cellular demise. Two cell lines were used, one with wild-type expression of three drug transporters, PDR1, PDR3, and SNQ2 (WT) and one cell line with PDR1, PDR3, and SNQ2 knocked out (KO). Using a modified MAGeCK analysis pipeline with suggested significance cutoffs (Li W, et al., Genome Biol. 2014; 15(12):554), numerous protein-drug interactions (e.g., HIV protease with atazanavir), and non-specific drug interactions (e.g., valproic acid with EYFP) were identified (see FIG. 10). In addition, the dose-response information with the expression of the HIV protease within the drug exporter KO background was captured showing rescue by 5 known HIV protease inhibitors as compared to 2 significant interactions for WT cells expressing HIV protease (see FIG. 10).

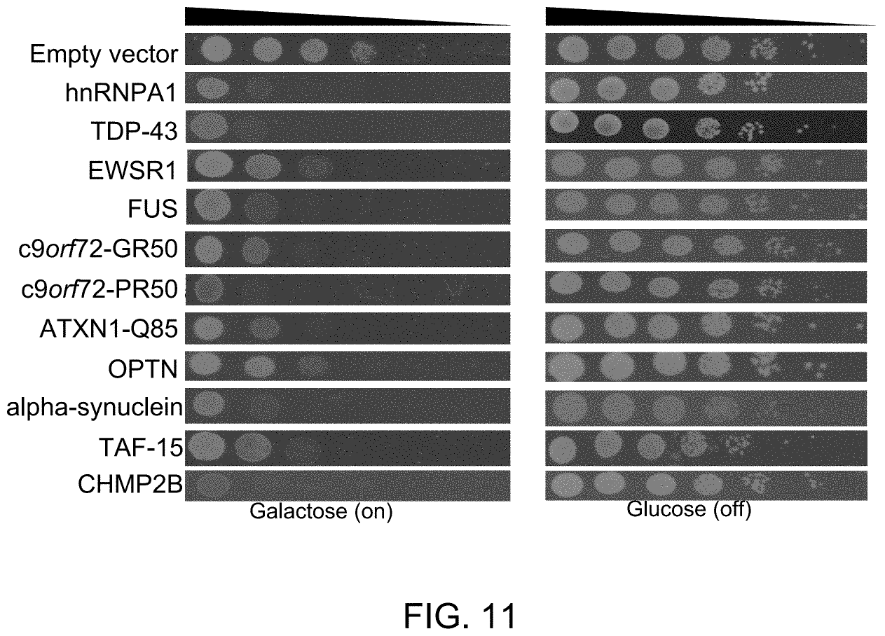

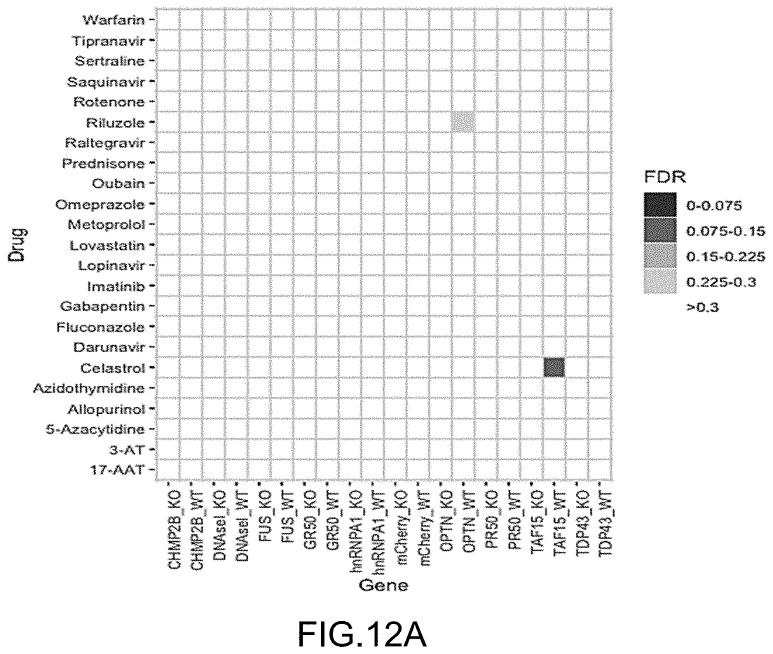

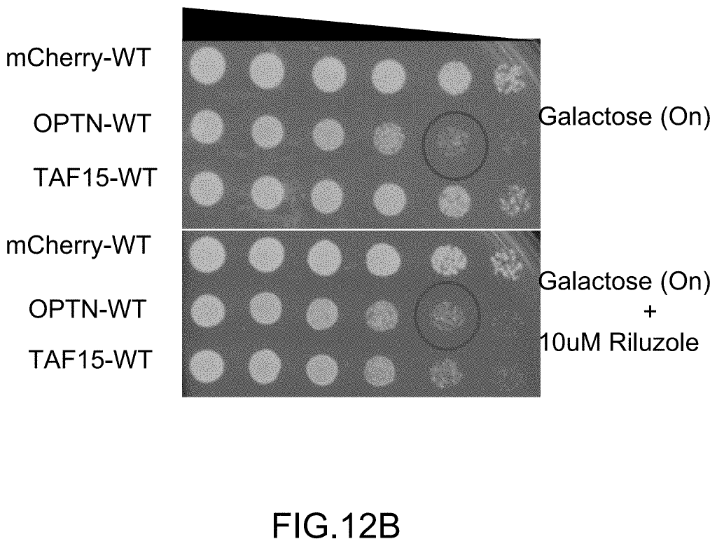

In addition, the presently disclosed screening methods were also applied towards an entirely orthogonal set of proteins. Aggregation-prone proteins are prevalent in numerous eukaryotic life forms and are implicated in a wide range of neurodegenerative diseases. Studies have demonstrated that expression of these aggregation-prone proteins in yeast results in toxicity and that this toxicity is a result of the propensity of these proteins to aggregate (Cooper A A, et al., Science 2006; 313(5785):324-8; Armakola M, et al., Nat Genet. 2012; 44(12):1302-9; Treusch S, et al., Science 2011; 334(6060):1241-5). As demonstrated in FIG. 11, expression of a wide range of aggregation-prone proteins of human origin within yeast resulted in toxicity to the yeast cell. In a drug screen of aggregation-prone proteins against 23 drugs, two significant interactions were identified (see FIG. 12A). One of two protein-interactions was subsequently verified and displayed (see FIG. 12B), further indicating that the disclosed methods are robust and can detect verifiable protein-drug interactions from a pool of potential interactions.

### Example 2: Identifying ALS Therapeutics Through Multiplexed Drug Discovery

Amyotrophic lateral sclerosis (ALS) is a heterogeneous neurodegenerative disease in which patients suffer from deterioration of both upper and lower motor neurons, leading initially to a progressive loss of voluntary movement and ultimately, death (Hardiman O, et al., Nat Rev Dis Primer 2017; 3:17071). Today more than 200,000 individuals are affected by this disease, and as the world's population continues to age these numbers are expected to further increase (Arthur K C, et al., Nat Commun 2016; 7:12408). The mean life expectancy of patients recently diagnosed with disease is less than 5 years, with no FDA-approved therapies that stop or reverse the disease course currently available (Hardiman 0, et al., Nat Rev Dis Primer 2017; 3:17071). To date, the molecular basis of ALS has remained elusive although recent efforts have revealed over a dozen proteins that when mutated increase the rate of disease within patients. A hallmark of many of these mutant proteins is an inherent propensity to form insoluble protein aggregates in cells (Brauer S, et al., J Neural Transm Vienna Austria 1996 2018; 125:591-613; Medinas D B, et al., Hum Mol Genet 2017; 26:R91-104). Remarkably, when these aggregation-prone proteins are expressed in the yeast S. cerevisiae, one of the most obvious phenotypes is a decrease in growth rate. This simple association between protein toxicity and cell growth has been exploited within yeast to identify genetic suppressors of the toxicity of ALS-associated TDP-43, FUS and C9orf72 dipeptide repeats, as well as other neurodegenerative disease-associated proteins such as amyloid-beta and α-synuclein (Armakola M, et al., Nat Genet 2012; 44:1302-9; Cooper A A, et al., Science 2006; 313:324-8; Jovicid A, et al., Nat Neurosci 2015; 18:1226-9; Treusch S, et al., Science 2011; 334:1241-5; Sun Z, et al., PLoS Biol 2011; 9:e1000614). Furthermore, the mammalian orthologues of the identified yeast suppressors also mitigate toxicity when tested in neuronal models, suggesting that hits discovered in yeast have a strong potential for translation (Cooper A A, et al., Science 2006; 313:324-8; Jovicid A, et al., Nat Neurosci 2015; 18:1226-9; Treusch S, et al., Science 2011; 334:1241-5; Sun Z, et al., PLoS Biol 2011; 9:e1000614). This is perhaps not surprising given the high degree of conservation between yeast and humans with regard to fundamental cell biological processes such as protein folding, sorting, modification and turnover (Tenreiro S, et al., J Neurochem 2013; 127:438-52; Tardiff D F, et al., Mov Disord Off J Mov Disord Soc 2014; 29:1231-40).

In the present Example, the association in yeast between aggregation-prone protein toxicity and growth rate is taken advantage of to create a scalable, high-throughput drug screening strategy. A multiplex screening platform is developed with the ability to determine specificity, off-target activity, and dose-response profile for each of the small molecules examined. In addition, the behavior of the present platform is tested and a series of small molecule library screens is performed, first analyzing a library composed of FDA-approved compounds, before turning to additional structurally diverse small molecule libraries. Hits that show activity across several unique ALS models are further assessed using iPSC-derived motor neurons from ALS patients. As a result, a set of potent small molecule leads can be identified with demonstrated biological activity within both yeast and human models of disease along with accompanying specificity, off-target, and dose-response data for each compound.

A. Developing A Multiplex Screening Platform With The Ability To Determine Specificity, Off-Target Activity, And Dose-Response Profile For Each Of The Small Molecules Examined

Methods

Strain Generation

A previously generated library of DNA barcoded yeast strains which contains over 1000 unique barcodes is used in the present study (Yan Z, et al., Nat Methods 2008; 5:719-25). To these DNA barcoded yeast strains, a LiAc-based transformation procedure is used to introduce either a galactose inducible aggregation-prone protein associated with ALS, a negative control protein, or a positive control protein (e.g. TDP-43, EYFP or HIV protease, respectively) (Alberti S, et al., Yeast Chichester Engl 2007; 24:913-9; Gietz R D. Methods Mol Biol Clifton N.J. 2014; 1205:1-12). To alter the levels of small molecule within the strains, the three major drug-exporters within yeast, PDR5, YOR1, and SNQ, are tested (Rogers B, et al., J Mol Microbiol Biotechnol 2001; 3:207-14). Using a Cas9-based genome engineering approach, variant strains are created. These variant strains accumulate more or less drug by either removing these transporters from the DNA barcoded strains or inserting a strong promoter upstream of these genes, respectively (DiCarlo J E, et al., Nucleic Acids Res 2013; 41:4336-43).

Curating a List of Yeast Mutants with Increased Sensitivity to Exogenous Stressors

To determine which yeast mutants should be employed to monitor for undesired off-target effects, a previously published dataset is analyzed in which 301 yeast mutants, selected to represent various key cellular pathways, are tested across >15,000 small molecules (Piotrowski J S, et al., Nat Chem Biol 2017; 13:982-93). These data are analyzed to determine the minimal number of mutant strains necessary to capture the majority of small molecules which show growth inhibition and hence would be representative of the undesired off-target activity that is aimed to be detected within the screens.

Verification of Strains

Overnight growth followed by serial dilution and plating to inducing and non-inducing conditions are employed to ensure that all disease models and controls show the proper behavior (e.g., strains overexpressing TDP-43 show decreased growth as compared to EYFP; positive control genes such as HIV protease show toxicity when induced as compared to EYFP). Yeast mutants designed as sensors for various exogenous insults are serial diluted and plated to either control media or media containing a drug known to inhibit their growth (e.g., rad52 mutants plated to media containing the DNA-damaging agent methyl methanesulfonate) to test that they show the expected sensitivity profile (Chavez A, et al., J Biol Chem 2011; 286:5119-25). For cells with altered expression of drug exporters, a similar serial dilution and plating assay is employed to show that strains in which the major drug exporters are removed or overexpressed show increased or decreased drug sensitivity, respectively. A set of 20 structurally distinct toxic small molecules (e.g., hygromycin, cyclohexamide, cisplatin) are tested and the growth of the exporter mutants is compared with respect to wildtype cells (Piotrowski J S, et al., Nat Chem Biol 2017; 13:982-93).

Discussion

DNA Barcodes in Combination with Precise Genetic Alterations Bypass Limitations of Scale and Assay Readout Common to Conventional Drug Screening Platforms

To capture the diverse mechanisms through which various genetic perturbations lead to ALS, a broad set of ALS models is assembled (see Table 2) (Ghasemi M, et al., Cold Spring Harb Perspect Med 2018; 8).

TABLE 2
List of ALS-associated proteins to be modeled within yeast.

| | | | | |
|---|---|---|---|---|
| TDP-43 | FUS | Alpha-synuclein | SUP35 | TMEM106B |
| HNRNPA1 | HNRNPA2B1 | Amyloid Beta | RNQ1 | ATXN1 |
| C9ORF72 | C9ORF72 | Htt | CHOPS- | PAB1 |
| (GR50) | (PR50) | | m8 | |
| OPTN1 | TAF15 | CHMP2B | PAB1 | Poly-Cystine |
| UBQLN2 | SOD1 | EWSR | ANG | Poly-Lysine |
| Poly-Glutamine | Poly-Alanine | | | |

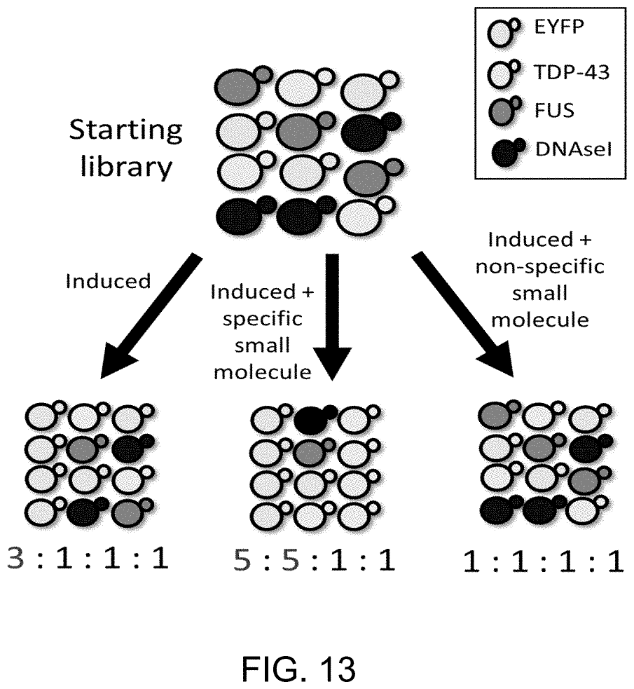

Each model consists of a yeast strain with a unique DNA barcode integrated within its genome and an ALS-associated aggregation-prone protein under the control of an inducible promoter (Armakola M, et al., Nat Genet 2012; 44:1302-9; Jovičić A, et al., Nat Neurosci 2015; 18:1226-9; Sun Z, et al., PLoS Biol 2011; 9:e1000614; Yan Z, et al., Nat Methods 2008; 5:719-25). Because each yeast strain contains a unique DNA barcode, barcode abundance is used as a surrogate for cell growth, and next-generation sequencing can be used to determine the abundance of all members of the pool (Delneri D. FEMS Yeast Res 2010; 10:1083-9). Yeast ALS models that experience improved growth in the presence of the tested small molecule (induced+specific small molecule) are enriched relative to the control situation (induced), providing a simple readout for determining which small molecules are candidate therapeutics (see FIG. 13). Furthermore, to reduce the effects of biological and technical noise, each disease model is placed within three different DNA barcoded strains, that are all simultaneously examined within the final mixed pool. By looking at the consensus behavior of all barcodes associated with the same ALS model, the ability to detect meaningful signal is further increased.

Assessing Small Molecule Specificity

To gain insight into the specificity of all screened small molecules, a set of DNA barcoded negative and positive control proteins is generated. The negative control proteins are designed such that they have no effects on growth within yeast (e.g., EYFP). The positive control proteins are selected to be detrimental to the growth of cells via entirely different mechanisms than the ALS models being examining (e.g., overexpression of HIV protease), such that small molecules which specifically rescue the ALS models would not be expected to also rescue the positive controls. In contrast, non-specific small molecules that rescue the ALS models through indirect mechanisms (e.g., by inhibiting the inducible expression system) are also expected to rescue the positive control proteins and therefore are readily discerned during the screening process described in the present disclosure (see FIG. 13, induced+non-specific small molecule).

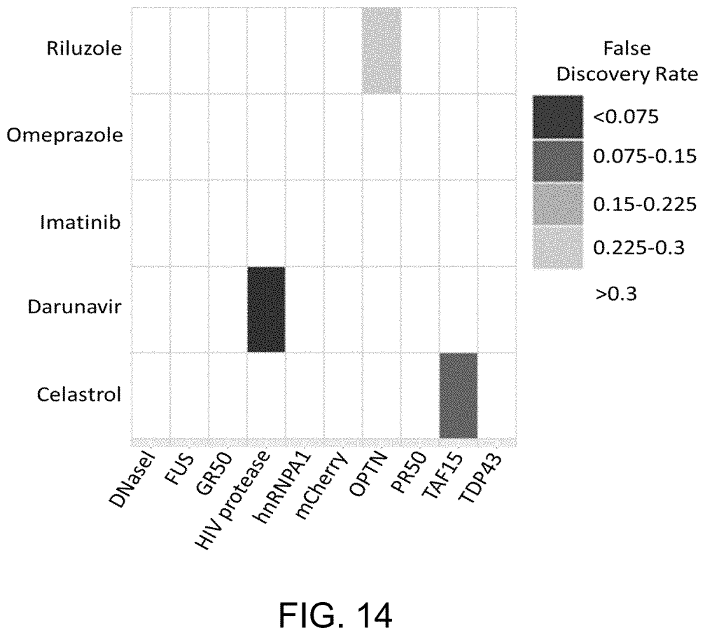

As demonstrated in FIG. 14, the barcoding system performed as expected, enabling several disease models and controls to be simultaneously probed.

Monitoring for Off-Target Effects

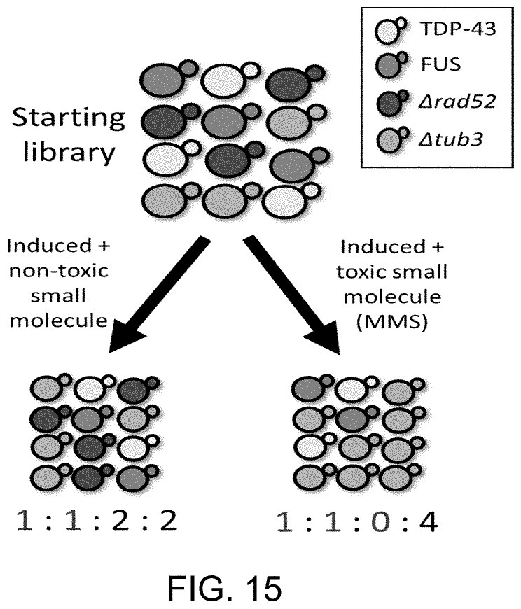

Test compounds with undesired off-target effects are detected by including a series of barcoded yeast strains that are designed, through genetic mutation, to be highly sensitive to various cellular insults, such as DNA damage or mitotic blockade (see FIG. 15) (Piotrowski J S, et al., Nat Chem Biol 2017; 13:982-93). If a tested small molecule shows depletion of members of the off-target DNA barcoded yeast, it is given a lower priority during follow-up studies since the compound is likely to have undesired pleiotropic effects.

Gaining Dose-Response Information

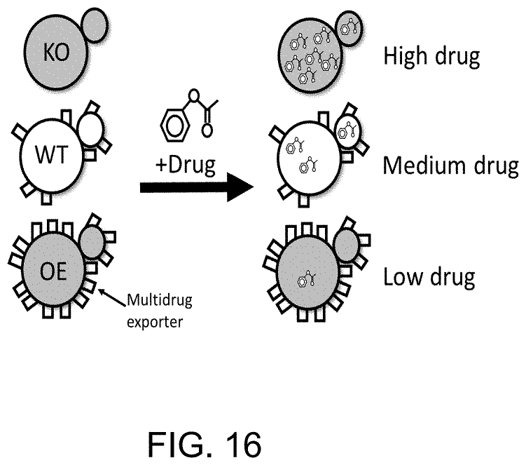

Most screens are performed at a single drug concentration, typically between 1-10 μM, to minimize labor and costs (Blass B E. Basic principles of drug discovery and development. 2016). Screening at a single concentration prevents the identification of drugs with therapeutic effects outside of the tested range or with effects that change at different drug concentrations (e.g., drug rescues at low concentrations and is toxic at high concentrations). Furthermore, a commonly-desired feature of lead candidates is a clear dose-response relationship, as this is thought to indicate a direct relationship between the small molecule and its therapeutic target. Yet, conventional screening methods using single drug concentrations are not able to capture any dose-response information (Hughes J P, et al., Br J Pharmacol 2011; 162:1239-49). To overcome this limitation, a series of yeast strains is engineered with varying expression levels of the three main multi-drug exporters (PDR5, YOR1, SNQ2) within S. cerevisiae in order to modulate the steady-state concentration of drug in each cell (see FIG. 16) (Rogers B, et al., J Mol Microbiol Biotechnol 2001; 3:207-14). A prior study that modulated drug transporter levels found that strains with modified drug transporter levels had phenotypes upon exposure to 35% of all tested compounds compared to 7% for wild-type cells (Piotrowski J S, et al., Nat Chem Biol 2017; 13:982-93). Of note, the lack of observed phenotype with the other 65% of compounds tested does not imply that they were not able to enter into yeast, the molecules could simply have been benign to yeast growth. By examining the ALS models within wild type cells and cells either lacking or overexpressing several of the major drug exporters, dose-response information is obtained during the primary screen and this information is useful to help triage the hits subsequently.

During the screens, yeast mutants are utilized which are designed to sense compounds with undesired off-target effects. These mutants capture many drugs with deleterious side-effects, but given the large scale of the screen (>10 ALS models by >10,000 compounds), they help better triage the large number of hits obtained. If additional off-target data are desired for any tested compound, gene expression analysis can be performed in the presence of the drug as compared to control conditions, using either the yeast models or iPS neuronal models described below.

In addition to work with these drug exporter mutants, additional yeast drug exporters (e.g., PDR10, PDR11, PDR15) are also manipulated, and the effects of expressing human small molecule importer/exporters (e.g., OAT1 and MDR1) within yeast are also tested to help further modulate drug levels within the barcoded yeast cells (Rogers B, et al., J Mol Microbiol Biotechnol 2001; 3:207-14; Nigam S K, et al., Physiol Rev 2015; 95:83-123; Robey R W, et al., Nat Rev Cancer 2018; 18:452-64).

B. Small Molecule Library Screens and Hit Validation within iPS-Cell Derived Motor Neurons.

By simultaneously screening several disease models at a time and collecting data on specificity, off-target effects and dose-response behavior, small molecule regulators of ALS with a higher chance of successful clinical translation are efficiently identified.

Methods

Performing Mixed Pooled DNA Barcoded Screening

Overnight cultures of DNA barcoded strains are mixed at an equal ratio and diluted 1:500 into fresh media containing inducer to activate the expression of their associated ALS or control genes, along with either vehicle control (DMSO) or 10 μM of a given small molecule compound. Twenty-four hours later, DNA is extracted from each well using a simple and cost effective LiAc/SDS based approach (Lõoke M, et al., BioTechniques 2011; 50:325-8). DNA barcodes from each well are then selectively amplified by PCR and all barcodes amplified from a single well are uniquely indexed allowing thousands of wells to be pooled and sequenced within a single next generation sequencing run (Piotrowski J S, et al., Nat Chem Biol 2017; 13:982-93).

Statistical Plan and Analysis Pipeline for Screening Data

To identify ALS model-drug interactions, statistically significant changes in barcode abundances are detected between control conditions (DMSO) compared to drug conditions (10 μM small molecule). Prior studies have demonstrated that existing RNA-seq algorithms that employ negative binomial models accurately identify changes in barcode abundance when sequencing count data is collected from barcoded yeast strains (Robinson D G, et al., G3 Bethesda Md2014; 4:11-8). However, RNA-seq algorithms assume each barcode corresponds to a separate gene. A strength of the present platform is the ability to attribute multiple barcodes to a single model thus improving the confidence to identify meaningful interactions. MAGeCK, an algorithm originally developed for analyzing CRISPR/Cas9 screening data, also uses a negative binomial model to identify changes in sgRNA abundance. It then compiles information from multiple sgRNAs against a single gene to then decide if the gene plays a role in the biology being investigated (Li W, et al., Genome Biol 2014; 15:554). This multiple sgRNA per gene relationship is equivalent to the multiple DNA barcodes per individual ALS model relationship described in the present disclosure. Thus, by adapting the MAGeCK pipeline to the data, the behavior of all DNA barcodes associated with a single model is taken into account and their collective behavior is used to detect statistically significant enrichment and depletion of ALS models after exposure to a particular compound.

Confirmation Testing

All small molecule hits being pursued for confirmation testing have fresh solutions made by purchasing new stocks of the chemical from a supplier. A 7-point dose-response curve for each hit against all ALS disease models and controls is performed examining drug effects at concentrations between 1 nM and 10 μM (Hafner M, et al., Curr Protoc Chem Biol 2017; 9:96-116). These confirmation studies are performed individually using conventional OD600 measurements of growth rate to rule out any potential false positive artifacts due to the multiplex screening approach. All testing is performed in triplicate on three separate occasions to ensure consistency of results.

iPS ALS Disease Models

iPS cells from healthy controls, familial, or sporadic ALS patients are differentiated into motor neurons via embryoid body formation. Motor neuron culture purity is tested via gene expression analysis (RNA-seq) and antibody staining (e.g., Tuj-1, HIB9) (Sances S, et al., Nat Neurosci 2016; 19:542-53). Cells are treated with 1-100 mM of L-glutamate or 5-500 μM arsenite±small molecule identified from screen for 4 hours and then analyzed for live/dead staining by incubating cells with 1 mM calcein AM and 1 mM propidium iodide for 30 minutes before imaging (Donnelly C J, et al., Neuron 2013; 80:415-28; Egawa N, et al., Sci Transl Med 2012; 4:145ra104). Images are quantified using Image J in combination with custom scripts. All experiments are performed in triplicate.

Discussion

Validation of Large-Scale Pooled Screening Approach

Upon generating the final DNA barcoded library, its behavior is validated by pooling the hundreds of barcoded strains together and growing the mixture in the presence of small molecules with known effects such as arsenite and Darunavir, which are expected to influence the growth of the DNA barcoded ALS models or control HIV protease expressing cells, respectively (Leggett C, et al., J Neurol Sci 2012; 317:66-73; Orru S, et al., Hum Mol Genet 2016; 25:4473-83; Surleraux DLNG, et al., J Med Chem 2005; 48:1813-22). After the validation step, a small scale screen is performed using a set of 1496 FDA-approved small molecules, as hits obtained from this drug library have a much quicker pathway to patient testing given that their side-effect profiles and long-term effects are generally well understood (Ashburn T T, Thor K B. Nat Rev Drug Discov 2004; 3:673-83). The FDA-approved compound library is also used to help refine the screening parameters and to test issues of assay noise and reproducibility. Finally, this library is used to verify that identified hits translate well when tested one at a time using conventional approaches.

Large-Scale Screen and Confirmation Testing of Small Molecule Regulators of ALS Disease-Associated Proteins

After completing the initial screen, the system is probed with a more complex chemical library such as a 10,000+ member Lead Optimized Compound library. Each compound within the library represents a unique scaffold that is well suited for further lead development. Of note, because the multiplex nature of the present approach, by performing a single 10,000 molecule screen, data for 100,000 drug-ALS disease model interactions, and the off-target effects and dose-response interactions are also collected and quantified. Hits that show specific rescue of two or more ALS models, have no effect on off-target models and show clear dose-response behavior are prioritized for further investigation. This confirmation screen consists of a 7-point dose-response curve with each hits individually tested against all ALS models along with positive and negative controls to characterize if hits are specific to a particular ALS model or generic in their rescue (Hughes J P, et al., Br J Pharmacol 2011; 162:1239-49; Blass B E. Basic principles of drug discovery and development. 2016). To control for errors in the annotation of the compound library, along with small molecule degradation or modification over time which may have occurred during library storage, each drug candidate is freshly obtained from outside vendors and new stock solutions are prepared (Hughes J P, et al., Br J Pharmacol 2011; 162:1239-49).

Secondary Testing within Human ALS Motor Neuron Models

Those candidates that pass confirmation testing are then assayed across a panel of human iPS-cell-derived motor neurons from healthy control and sporadic and familial ALS cases. Hits are tested for their ability to affect neuronal survival in the context of arsenite-induced oxidative stress and glutamate excitotoxicity, both of which have been shown to cause preferential toxicity to iPS-cell-induced motor neurons derived from ALS patients (Lee S, Huang E J. Brain Res 2017; 1656:88-97). In particular, how the hits identified in yeast translate to their corresponding human ALS model (e.g., yeast expressing TDP-43 and motor neurons containing TDP-43 mutations) are studied in detail. By examining the small molecule hits across a panel of sporadic and familial ALS cases, compounds that target conserved mechanisms of disease are identified, and are beneficial to a variety of ALS patients (Fujimori K, et al., Nat Med 2018; 24:1579-89).

The multiplexed barcode strategy as described in the present disclosure is amenable to considerable scaling. When very large libraries are screened, it can be beneficial to sequence the libraries at higher depth to increase the accuracy in detecting changes in abundance or reducing the number of DNA barcoded cells screened within a given experiment.

Since the screen is being performed in multiplex, a large number of small molecule hits are obtained. To help triage the hits, more stringent false discovery rates (FDR) are set, focusing on those which show the largest fold-change in growth rate, grouping molecules with similar structure and only pursuing a single representative example, concentrating on hits which rescue the most ALS models and prioritizing compounds which might already have clinical data (e.g., FDA approved compounds). On the other hand, when few hits are produced from the screens, additional small molecule libraries can be screened, or the FDR threshold can be decreased. Finally, while the method requires next generation sequencing (NGS) as a readout, the increase in cost is minimal as only 5 million reads are required per 96 conditions tested.

Based on the results of these studies, the top drug candidates can be investigated for activity, specificity, bio-distribution, absorption, metabolism, and excretion (Hughes J P, et al., Br J Pharmacol 2011; 162:1239-49). Subsequently, lead compounds are tested in preclinical animal studies and then human clinical trials.

### Example 3: Discovering Precision Small Molecule Therapeutics Through The Application of Multiplex Drug Screening Technologies

The present disclosure describes a strategy for identifying small molecule therapeutics that encapsulates significant advantages over current approaches. First, the present disclosure enables screening of small molecule inhibitors to several dozen proteins at a time. Second, the present disclosure provides information on the specificity and potential off-target activity of every small molecule examined. Third, a range of drug concentrations can be tested simultaneously within a single screen. This new approach can be used to identify a series of first-in-class high specificity kinase inhibitors, which serve as both the validation of the approach and valuable assets for use in future preclinical/clinical trials.

The present example proceeds in two phases. In the first phase (phase I), a human kinase library is established, platform components are optimized, and a series of pooled barcode screening experiments are performed using known kinase inhibitors to establish that the platform is able to detect the activity of already characterized kinase inhibitors. In the second phase (phase II), small molecule screens are performed to identify specific and potent kinase inhibitors, along with full validation of the results from the screens using orthogonal assays. At the completion of the studies, a generalizable drug screening platform is developed, with proven potential to identify small molecules with increased specificity and decreased toxicity, and a set of compounds with specificity against various human kinases are identified for downstream preclinical/clinical testing.

Phase I. Establish Human Kinase Library, Optimize Platform Components, and Perform a Series of Pooled Barcode Screening Experiments Using Known Kinase Inhibitors.

Basis of System and Strategy to Enable Multiplexing

The present drug screening platform is based on the observation that when an enzyme is overexpressed within the yeast S. cerevisiae, it often causes a profound growth defect that is dependent upon the enzyme's catalytic activity. Furthermore, if the enzyme's function is blocked by the application of a small molecule inhibitor, growth of the yeast is restored. This restoration in cell growth is a clear and easily quantifiable phenotype.

This Example is focused on identifying inhibitors for kinases, as previous literature has established that the expression of human kinases within yeast causes a growth defect that can be rescued by the application of existing inhibitors. Furthermore, kinases represent attractive drug targets as they play critical roles in countless biological processes (e.g., cancer, autoimmunity), and have a history of being targeted for therapeutic benefit.

Despite the frequency with which kinase inhibitors are used clinically, nearly all are active against more than their intended target, with some able to inhibit upwards of several dozen kinases. This near universal promiscuity contributes to the numerous side effects associated with this class of compounds, with almost half of all clinically available kinase inhibitors having FDA issued black box warnings for major complications such as hepatotoxicity and fistula formation. Furthermore, even seemingly benign side effects, such as nausea, vomiting, fatigue and diarrhea can limit the ability of patients to complete their treatment course, and thus the clinical utility of these valuable compounds. The difficulty in identifying specific kinase inhibitors is due to the structural similarity among the hundreds of kinases within the human genome. This similarity makes screening small molecules against multiple kinases absolutely necessary to ensure specificity, yet not feasible to do at library scales with current approaches.

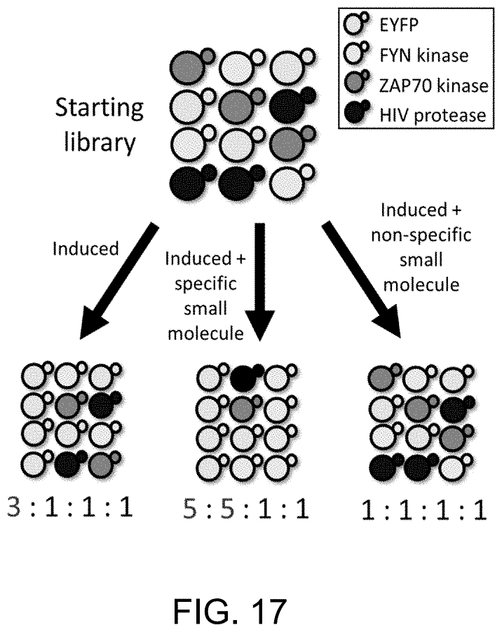

The present disclosure describes drug screening methods that enable the testing of dozens of kinases against a library of small molecules to identify a set of first-in-class high specificity kinase inhibitors. In the drug screening platform, each human kinase to be screened is placed under the control of an inducible promoter and transformed into a series of yeast strains that each contain a unique DNA barcode. Because each yeast strain contains a unique DNA barcode, different strains that each express a different kinase can be pooled and screened within a single well, and barcode abundance, which can be determined via next-generation sequencing, is used as a surrogate for cell growth of individual members of the pool. Yeast expressing a particular kinase that experience improved growth in the presence of the tested small molecule (induced+specific small molecule) are enriched relative to the control situation (induced), providing a simple readout for determining which small molecules are candidate inhibitors (see FIG. 17).

Assessing Small Molecule Specificity

To gain insight into the specificity of all screened small molecules, a set of DNA barcoded yeast strains expressing negative and positive control proteins is generated. The negative control proteins are designed such that they have no effect on growth within yeast (e.g., EYFP). The positive control proteins are selected to be detrimental to cell growth via entirely different mechanisms from the kinases being examined (e.g., overexpression of HIV protease), such that small molecules which specifically rescue the kinase expressing cells would not be expected to also rescue the positive controls. In contrast, non-specific small molecules that rescue the kinase expressing cells through indirect mechanisms (e.g., by inhibiting the inducible expression system) are also expected to rescue the positive control models and are readily discerned during the screening process (see FIG. 17). Finally, as multiple kinases are examined simultaneously within each test, specificity of any given compound can be determined with respect to a single kinase or to a broader class of kinases.

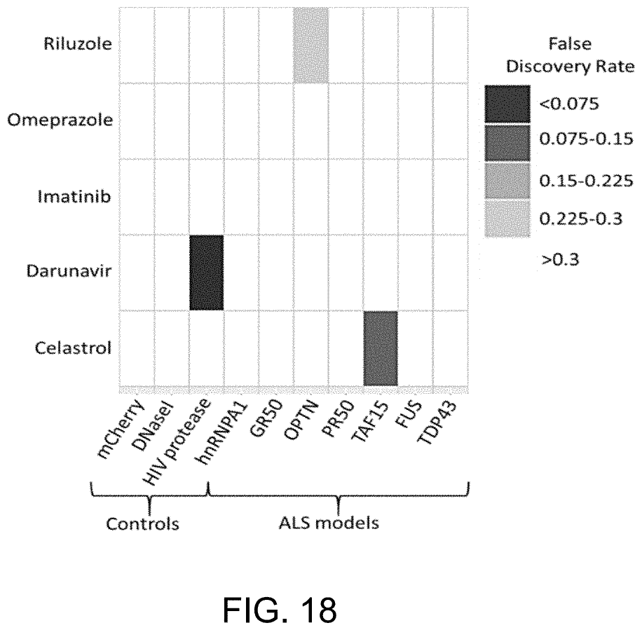

As demonstrated in FIG. 18, small molecules screened against yeast models of amyotrophic lateral sclerosis (ALS) showed that the present barcoding system behaved as expected, enabling several models and controls to be simultaneously probed.

Monitoring for Off-Target Effects

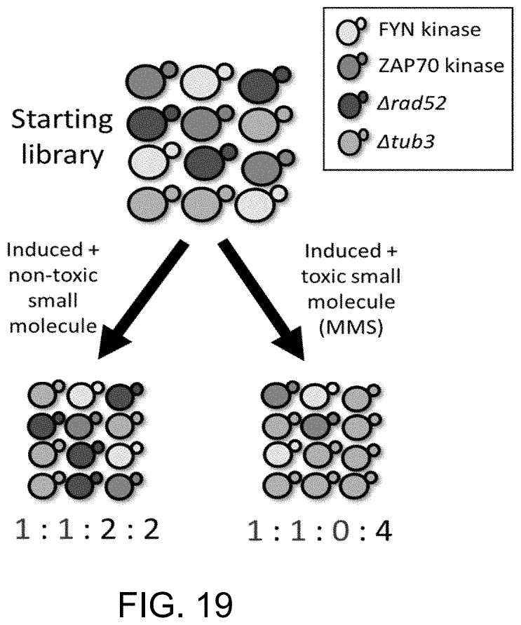

Compounds with undesired off-target effects are also detected within the system by including a series of barcoded yeast strains that are designed, through genetic mutation, to be highly sensitive to various cellular insults, such as DNA damage or mitotic blockade (see FIG. 19). To determine which yeast mutants should be employed as sensors of undesired off-target effects, a previously published dataset was analyzed in which 310 yeast mutants, selected to detect perturbation in key cellular pathways, were tested across >15,000 small molecules. The analysis indicates that using a set of 15 mutant strains identifies >50% of all compounds which showed cellular toxicity in the previous study. If a tested small molecule shows depletion of members of the off-target DNA barcoded yeast, this is taken into account when deciding which molecules to pursue for the follow-up studies since the compound is likely to have undesired pleiotropic effects that will limit its clinical utility.

Gaining Multiple Dose Testing within a Single Screen

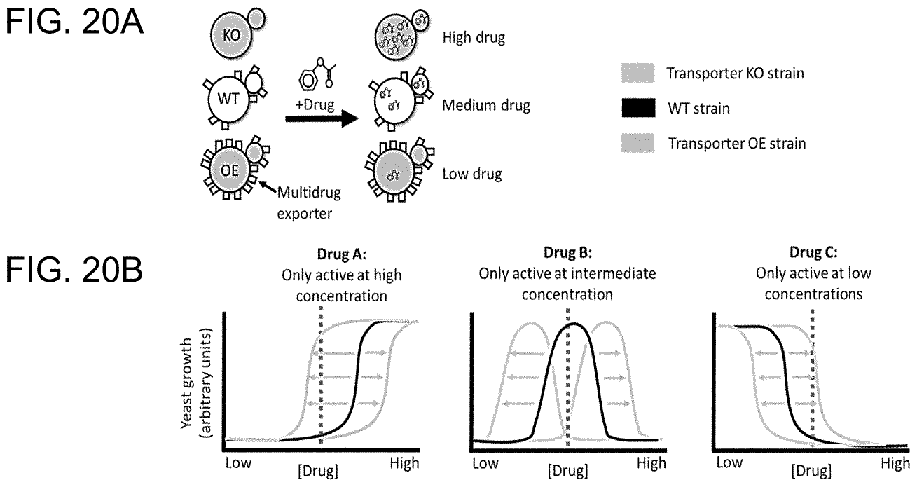

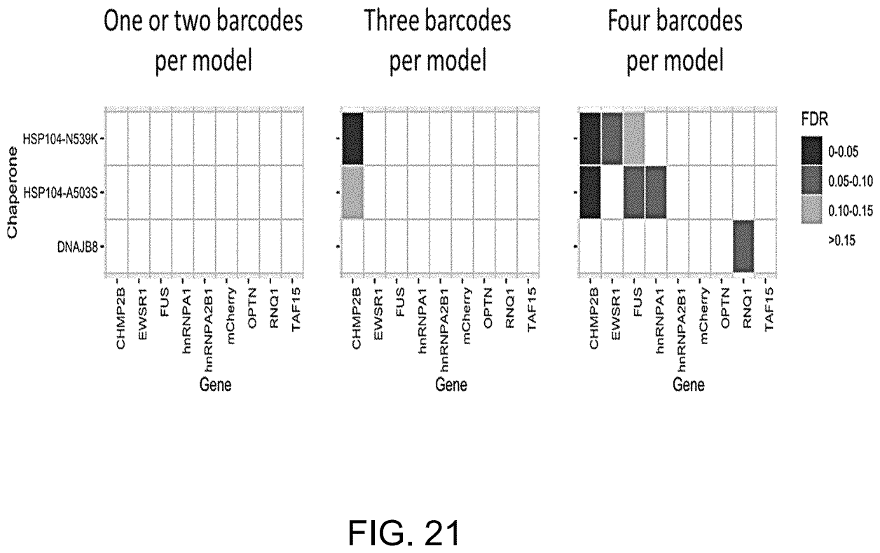

Most published screens have been performed at a single drug concentration, typically between 1-10 μM, to minimize labor and costs. Screening at a single concentration prevents the identification of drugs with therapeutic effects outside of the tested dosage or with effects that change at different drug concentrations (e.g., drug rescues disease model at nanomolar concentrations and is toxic at normally tested micromolar concentrations). Yet, conventional screening methods that test a single drug concentration are not able to capture this information, while the assay design of the present disclosure can. This is accomplished by using a series of engineered yeast strains that either overexpress or lack the three main multi-drug exporters (PDR5, YOR1, SNQ2) within S. cerevisiae, rendering these modified strains either extremely adept or inefficient at pumping out small molecules, respectively (see FIG. 20A-FIG. 20B). This approach gives some cell lines in the mixed pool a low intracellular drug concentration while other cell lines in the mixed pool receive a high intracellular drug concentration when exposed to the same extracellular drug concentration. For the screen, each kinase and control are added into the three different drug transporter backgrounds and DNA barcoded. The DNA-DNA DNA barcodes helps keeping track not only of what kinase the cell contains, but also the status of the cell's ability to export drugs. Thus, when cells are exposed to a single concentration of drug, the effects of screening each kinase at three drug doses are mimicked without additional cost or labor, as all strains are pooled and mixed together during screening. Using the information obtained, interesting lead compounds are identified such as those that show no sign of toxicity at higher intracellular doses (i.e., within strains that lack exporter expression) and maintain activity even at lower intracellular doses (i.e., within strains that overexpress the major drug exporters). Compounds that show toxicity at higher concentrations or lack of efficacy at lower concentrations still represent interesting starting points for later medicinal chemistry efforts, which would be particularly worthwhile if these compounds were found to have other desired properties such as specificity to only a single kinase.

Redundant Barcoding to Improve Data Quality

When screening at such high scale, it is critical to take issues of technical variation (e.g., library bias introduced during PCR amplification of DNA barcodes) and biological variation (e.g., cells accumulating suppressor mutations) into consideration. To help mitigate these effects, a novel redundant barcoding approach was developed that places each of the combinations between drug transporter background and kinase/controls/off-target detectors into 4 different uniquely barcoded strains. By looking at the collective behavior of all 4 independently barcoded cells associated with the same genotype (e.g., FYN kinase expression, with drug exporters knocked out), technical and biological variations are under a better control, as it is unlikely that by chance all 4 barcoded cell lines would exhibit the same technical or biological deviation. As demonstrated in FIG. 21, the use of redundant barcoding was strongly supported.

Validation of Pooled Screening Approach

Upon generating a final DNA barcoded library (see FIG. 22), its behavior is validated within a pilot screen by combining the hundreds of barcoded strains into a single pool and growing the mixture in the presence of small molecules with known effects such as the ceramide kinase inhibitor NVP-231 or the FYN kinase inhibitor AZD0530.

Results and Discussion

Sets of drug off-target detector strains have been developed and their behaviors were validated. A set of yeast chassis that accumulate more or less drug by manipulating the expression of key multidrug exporters in yeast was created, and the optimal number of redundant barcodes for screening at high multiplex was determined. In addition, series of experiments using a library of at least 100 barcoded strains were performed in combination with known kinase inhibitors to show system behaves as expected.

The feasibility and benefit of simultaneously performing in-screen controls for compound specificity and off-target activity was demonstrated, along with enabling cells to sample multiple drug concentrations within a single assay. Furthermore, the novel application of redundant barcoding within the context of HTS allows the collection of robust data while operating at much greater scales than those of conventional drug screening paradigms. These studies produce results that are directly applicable to the discovery of kinase inhibitors and broadly applicable to the design of HTS platforms.

Subsequently, for Phase 1, a highly multiplexed strategy for drug screening will be generated. Specifically, a library of human kinases is first generated with compatible expression in yeast. Then any toxicity of these kinases is determined when expressed in yeast. If kinase expression alone does show toxicity, then any particular yeast mutants that were sensitized to a given kinase's expression are determined.

Phase II. Perform a Set of Small Molecule Screens to Identify Specific and Potent Kinase Inhibitors, Validate the Results of the Screens

Pooled Screen with FDA-Approved Compounds

Once the validation studies are complete (Phase 1), a small scale screen is performed using a set of 1496 FDA-approved small molecules, as hits obtained from this drug library have a much quicker pathway to patient testing given their existing use within clinical settings and an abundance of knowledge with regard to their pharmacodynamic and pharmacokinetic properties. The FDA-approved compound library is also used to help refine screening parameters and test issues of assay noise and reproducibility. Finally, this library is used to verify that identified hits translate well when tested one at a time using conventional OD600 measurements of growth.

Large-Scale Screen for Kinase Inhibitors

After completing the initial screen, the system is probed with a more complex chemical library such as the 10,000+ member Lead Optimized Compound library. Each compound within the library represents a unique chemical scaffold that is well suited for further lead development, and pre-filtered for traditional physicochemical descriptors such as the rule of five, rotatable bond count, topological polar surface, and suitable aqueous solubility. Of note, because of the multiplex nature of this approach, by performing a single 10,000 molecule screen, data for 300,000+ drug-kinase interactions are obtained, along with hundreds of thousands of data points quantifying potential off-target effects and effects of drug concentration for all compounds tested. As drug screens typically produce hit rates between 0.01%-1%, the majority of compounds are expected to show no activity against the kinases, enabling a rapid ruling out of a large set of non-kinase relevant compounds and, thus, the much smaller subset of promising molecules were focused for subsequent downstream validation experiments. Validation testing is performed on at least 100 hits from the screen, altering the selection criteria as necessary to obtain the initial list of compounds. Hits are prioritized to show specific inhibition of a single kinase, have limited effects on off-target models, and show rescue within multiple drug exporter backgrounds. In contrast to the initial screen which is performed with a single drug concentration, the confirmation testing consists of a 7-point dose-response curve with each hit from the screen tested against the kinase they rescued.

Secondary Testing Using In Vitro Kinase Assays

Those small molecule candidates that pass confirmation testing are examined in vitro against their target kinase. Compounds that are found to inhibit kinase activity in vitro are then further tested in vitro against an expanded set of kinases that are most similar to the target kinase. For small molecules that continue to show high levels of specificity, a subset of them are tested in vitro against the majority of human kinases, to fully confirm their specificity.

Discussion

In Phase II, the multiplex screening strategy is applied to test >11,000 compounds, identifying those with the ability to inhibit kinase activity. Specifically, small molecule screens are performed against FDA-approved compound library and larger diversity oriented compound library. Results of multiplex screen are verified using traditional OD600-based approaches. A subset of the identified hits are tested individually within yeast to validate their behavior, followed by extensive in vitro testing.

### Example 4. Detection of Protein-Drug Interaction in a Mixed Pool with Simultaneous Multi-Dose Testing

A single drug screen was performed on a mixed pool of yeast cells harboring various target proteins in order to identify compounds that interfere with each of the tested protein's function. To perform the screen, a collection of target proteins were cloned into inducible yeast expression vectors. When yeast transformed with such vectors are exposed to conditions that induce expression of the target protein, the proteins cause toxicity to the yeast cell, resulting in a growth defect. The addition of a drug that inhibits the catalytic function of the target protein rescues the growth suppression and is the basis of the screening strategy as described in the present disclosure. In order to test multiple protein targets at a time, a unique DNA barcode was added to each yeast cell, such that when read using next generation sequencing, its abundance within a mixed pool can be quantified.

Protein-Drug Interaction Detection in a Mixed Pool with Simultaneous Multidose Testing

Yeast contain a number of multidrug transporters that export a large variety of exogenously applied compounds. By manipulating the expression of endogenous multidrug drug exporters or introducing into yeast additional multidrug importers or exporters, the amount of compound within each of the yeast cells can be regulated. As DNA-barcoding allows simultaneous testing of multiple yeast models at a time, by placing the same target protein within a background lacking drug exporters or containing normal levels of these exporters, how a given compound interacts with the target protein of interest when present at elevated or normal, intracellular concentrations, respectively, can be investigated.

To illustrate the utility of this approach, two yeast strains were utilized, one deficient for the expression of multidrug exporters (e.g., Δsnq2, Δpdr1, Δpdr3), and one that expressed wildtype levels of various multidrug exporters. Subsequently, the same target proteins were placed into both backgrounds. Each cell line was DNA barcoded and mixed together, and a compound of interest was added. As proof of concept, proteases and kinases were tested.

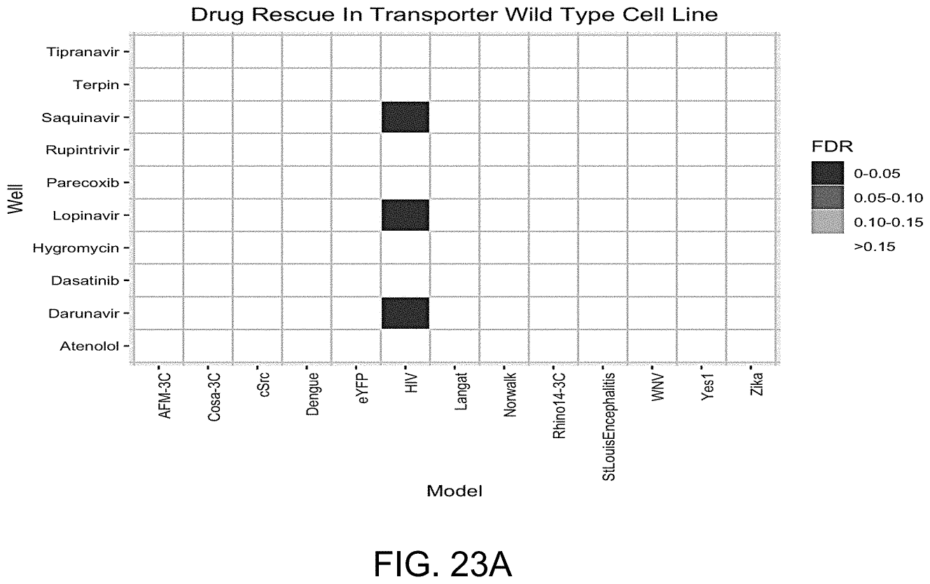

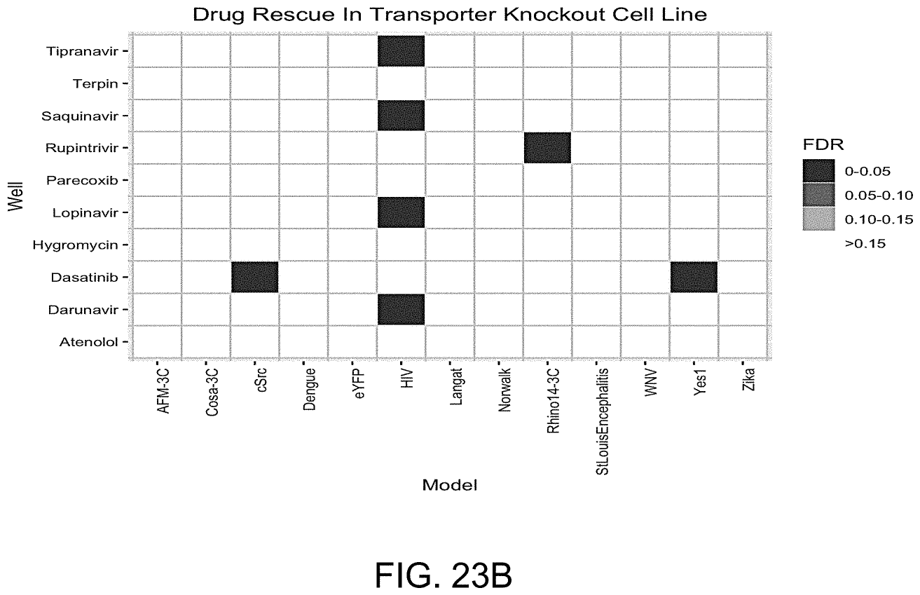

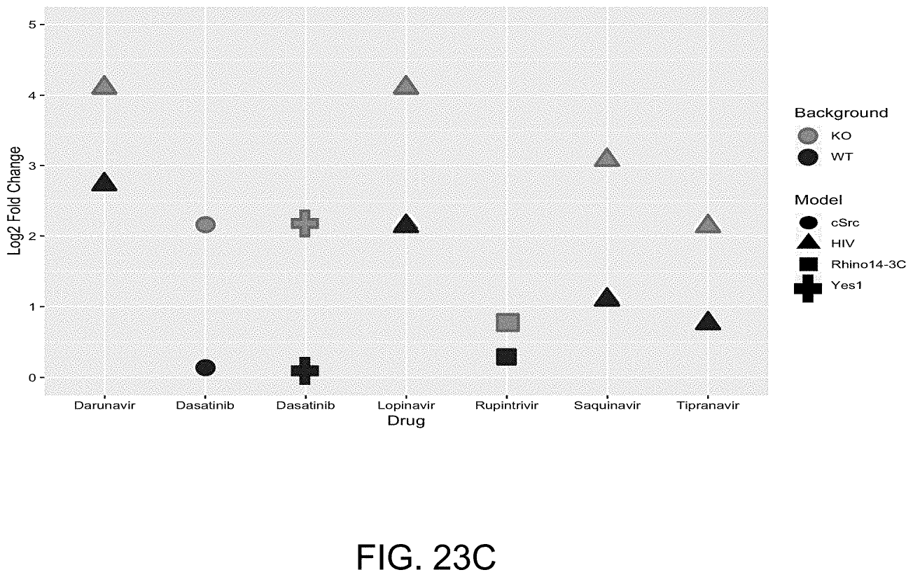

As demonstrated in FIG. 23A-FIG. 23C, cells that were deficient in drug exporters, leading to a higher intracellular concentrations of drug, were more likely to show rescue with compounds known to inhibit each of the proteases or kinases tested.

Redundant Barcoding Enables Detection of Interactions that would Otherwise be Missed.

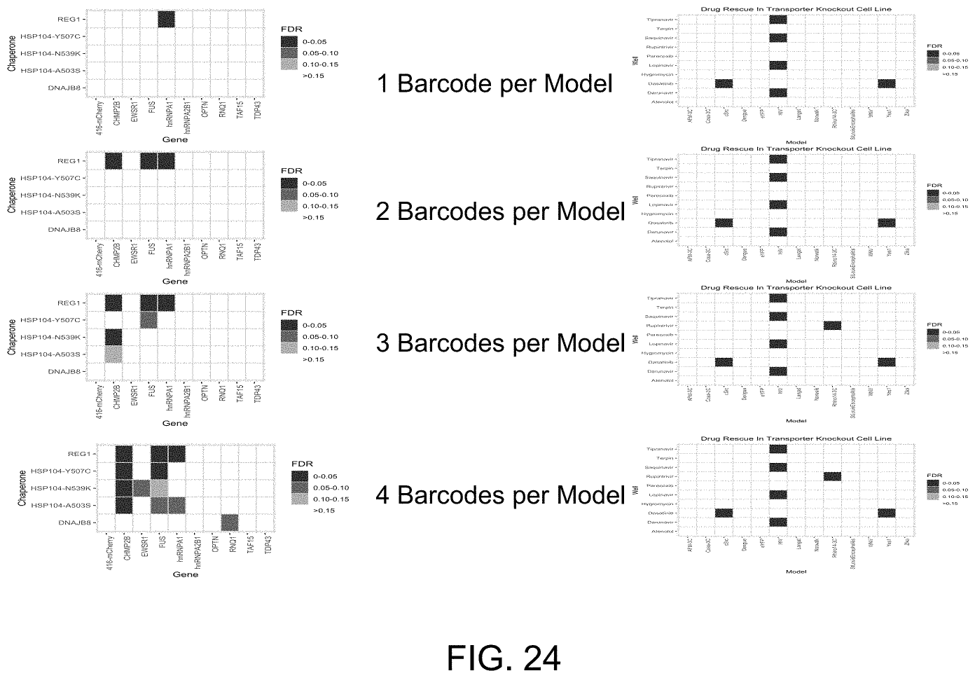

As DNA barcodes are infinitely scalable, a specific protein target of interest can be combined with a specific drug exporter background (e.g., wild type or knockout) and then introduced with different known barcodes to make redundant isogenic clones of that specific combination (e.g., HIV protease+drug transporter knockout). These isogenic clones allow for increased statistical power and sensitivity to detect interactions, as information can be pooled between clones to arrive at an overall statistic for the model (see FIG. 24).

Wimpy Yeast Allow for Detection of Drugs with Off-Target Effects.

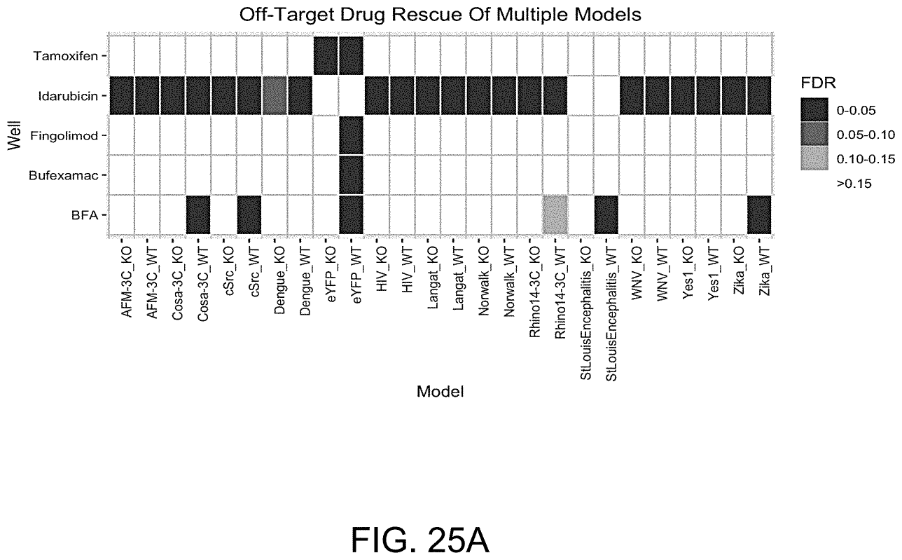

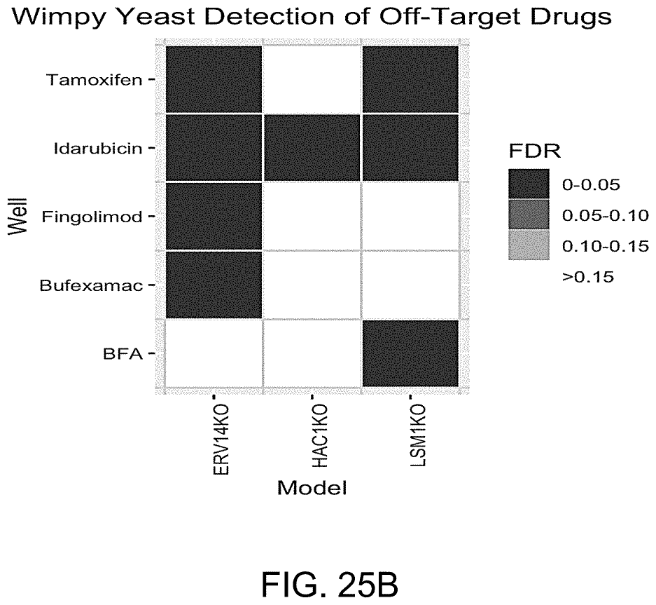

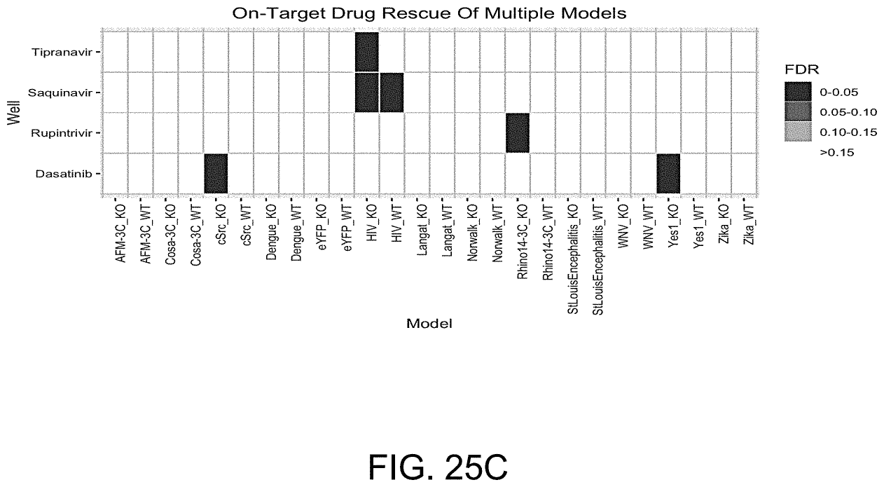

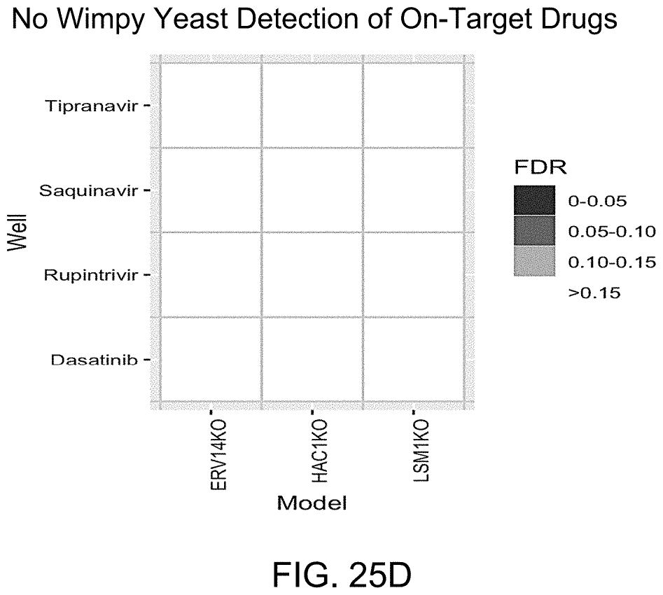

To detect drugs that have additional pleotropic effects, which may confound the interpretation by appearing to rescue specific models, but in fact was a result of off-target effects rather than on-target enzymatic inhibition, a series of “wimpy” yeast were developed that were engineered to be sensitive to a wide variety of off-target drug toxicities (i.e., perturbations to various biological pathways). These wimpy yeast strains are engineered by using genetic mutation to selectively render the cells hypersensitive to perturbations to various core cellular pathways. As previously mentioned within our disclosure, the selection of which mutations to use was based on analyzing previous literature such as those from the Boone lab (Piotrowski J S, et al., Nat Chem Biol. 2017; 13(9):982-993). By examining the behavior of these wimpy yeast, several compounds, such as idarubicin, showed significant rescue of the protease and kinase expressing cells (see FIG. 25A), but also showed a decrease in the growth of the wimpy models (see FIG. 25B). These results suggest that effects of compounds such as idarubicin are through a general alteration in yeast growth, likely through growth suppression, thus creating false positive signals in the assay. However, using the wimpy yeast models, such false positive hits could be easily identified and ruled out early. In contrast to non-specific compounds, drugs whose mechanism of action is more direct, such as several known protease or kinase inhibitors, showed minimal perturbation to the growth of the wimpy yeast, despite showing strong rescue for their specific protein target (see FIG. 25C and FIG. 25D).

1. A method of screening for an agent capable of specifically inhibiting a toxicity protein in a cell, the method comprising the steps of:
(a) providing a library comprising a plurality of proliferating cell types in the presence or absence of the agent, wherein each cell type comprises one or more inducible toxicity protein, wherein each proliferating cell type comprises a unique associated barcode, and wherein the library further comprises one or more control cell types selected from: (1) a proliferating cell type comprising a negative control inducible non-toxic protein, (2) a proliferating cell type comprising a positive control inducible toxicity protein, and (3) one or more proliferating cell types highly sensitive to cellular insults, wherein each control cell type comprises a unique associated control barcode;
(b) inducing the expression of each toxicity protein and the one or more control proteins;
(c) determining the relative number of unique associated barcodes and the unique associated control barcodes after a period of cell proliferation; and
(d) comparing the relative number of unique associated barcodes to one or more of the unique associated control barcodes, to thereby determine the effectiveness and specificity of the agent on the one or more toxicity proteins.

2. The method of claim 1, wherein each of the steps are performed concurrently.

3. The method of claim 1, wherein the one or more proliferating cell types highly sensitive to cellular insults are cells that carry mutations or deletions in one or more genes selected from the group consisting of RAD, LEA1, CHO2, RFM1, LSM1, HOC1, ROM2, HAC1, SMY1, ABP1, ERV14, SNT1, PFA4, SSD1, GSF2, and CLB2.

4. The method of claim 1, wherein each proliferating cell type further comprises one or more cell surface drug transporter having varying levels of expression for identifying an effective dosage of the agent having the ability to inhibit cellular toxicity.

5. The method of claim 4, wherein each combination of an inducible toxicity protein and a level of expression of a cell surface drug transporter is associated with two or more unique barcodes within the cell types comprising said combination.

6. The method of claim 5, further comprising step e) pooling results of step d) from the cell types comprising the same combination of the inducible toxicity protein and the level of expression of the cell surface drug transporter.

7. The method of claim 1, wherein the agent is a small molecule.

8. The method of claim 1, wherein the toxicity protein is a kinase, a protease, an aggregation-prone protein, a viral integrase, a nucleic acid binding protein, a structural protein, a protein chaperone, phosphatase, small GTPase, ubiquitin ligase, DNA or RNA polymerase, caspase, hydrolase, ligase, oxidoreductase, transcription factor, cell adhesion molecule, cell junction molecule, isomerase, transferase, adapter protein, or a reverse transcriptase.

9. The method of claim 1, wherein the cell types are eukaryotic or prokaryotic cell types.

10. The method of claim 9, wherein the cell types are yeast cells.

11. A method of identifying an effective dosage of a drug having the ability to inhibit cellular toxicity in a cell, the method comprising the steps of:
(a) providing a library comprising a plurality of proliferating cell types, wherein each cell type comprises one or more inducible toxicity protein and having varying levels of expression of one or more cell surface drug transporter, and wherein each proliferating cell type comprises a unique associated barcode, and
wherein the library further comprises one or more control cell types selected from: (1) a proliferating cell type comprising a negative control inducible non-toxic protein, and (2) a proliferating cell type comprising a positive control inducible toxicity protein, wherein each control cell type comprises a unique associated control barcode;
(b) inducing the expression of each toxicity protein in the presence or absence of a single concentration of the drug, wherein the proliferating cell types contain different intracellular concentrations of the drug as a result of the varying levels of expression of one or more cell surface drug transporter;
(c) determining the relative number of unique associated barcodes after a period of cell proliferation; and
(d) comparing the relative number of unique associated barcodes to one or more controls, to thereby determine the effective intracellular concentration of the drug on the one or more proliferating cell types.

12. The method of claim 11, wherein each combination of an inducible toxicity protein and a level of expression of a cell surface drug transporter is associated with two or more unique barcodes within the cell types comprising said combination.

13. The method of claim 12, further comprising step e) pooling results of step d) from the cell types comprising the same combination of the inducible toxicity protein and the level of expression of the cell surface drug transporter.

14. The method of claim 11, wherein each of the steps are performed concurrently.

15. The method of claim 11, wherein the one or more additional proliferating cell types are cells that have been modified to be highly sensitive to one or more cellular insults.

16. The method of claim 15, wherein the one or more proliferating cell types highly sensitive to cellular insults are cells that carry mutations or deletions in one or more genes selected from the group consisting of RAD, LEA1, CHO2, RFM1, LSM1, HOC1, ROM2, HAC1, SMY1, ABP1, ERV14, SNT1, PFA4, SSD1, GSF2, and CLB2.

17. The method of claim 11, wherein the agent is a small molecule.

18. The method of claim 11, wherein the toxicity protein is a kinase, a protease, an aggregation-prone protein, a viral integrase, a nucleic acid binding protein, a structural protein, a protein chaperone, phosphatase, small GTPase, ubiquitin ligase, DNA or RNA polymerase, caspase, hydrolase, ligase, oxidoreductase, transcription factor, cell adhesion molecule, cell junction molecule, isomerase, transferase, adapter protein, or a reverse transcriptase.

19. The method of claim 11, wherein the cell types are eukaryotic or prokaryotic cell types.

20. The method of claim 19, wherein the cell types are yeast cells.
